(19) United States
(12) Patent Application Publication
(10) Pub. No.: US 2003/0167524 A1
(43) Pub. Date: Sep. 4, 2003

(54) Methods for the production of multimeric protein complexes, and related compositions

(76) Inventors: Gijs Van Rooijen, Alberta (CA); Steven Zaplachinski, Alberta (CA); Peter-Bernard Heifetz, San Diego, CA (US); Steven Briggs, Del Mar, CA (US); Bipin Kumar Dalmia, San Diego, CA (US); Greg Del Val, San Diego, CA (US)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(21) Appl. No.: 10/032,201

(22) Filed: Dec. 19, 2001

Related U.S. Application Data
(63) Continuation-in-part of application No. 10/006,038, filed on Dec. 4, 2001, now abandoned.
(63) Continuation-in-part of application No. 60/331,363, filed on Dec. 19, 2000.
(60) Provisional application No. 60/302,885, filed on Jul. 5, 2001.

(51) Int. Cl. A01H 5/00; C12N 5/04

(52) U.S. Cl. 800/281; 435/419

(57) ABSTRACT

Improved methods for the production of multimeric-protein-complexes, such as redox proteins and immunoglobulins, in association with oil bodies are described. The redox protein is enzymatically active when prepared in association with the oil bodies. Also provided are related nucleic acids, proteins, cells, plants, and compositions.

### RELATED APPLICATIONS

Benefit of priority under §119(e) is claimed to U.S. provisional application Serial No. 60/302,885, filed Jul. 5, 2001, to van Rooijen, et al., entitled “METHODS FOR THE PRODUCTION OF REDOX PROTEINS”. This application is a continuation-in-part of U.S. utility application Ser. No. 10/006,038, filed Dec. 4, 2001 to van Rooijen, et al., entitled “METHODS FOR THE PRODUCTION OF REDOX PROTEINS”, which is a continuation-in-part of U.S. utility application Ser. No. 09/742,900, filed Dec. 19, 2000 to Heifetz, et al., entitled “METHOD OF PRODUCTION AND DELIVERY OF THIOREDOXIN”. This application is also a continuation-in-part of U.S. utility application Ser. No. 09/742,900. The subject matter of each of the provisional and utility applications is incorporated herein by reference in its entirety.

This application is related to International PCT application No. (attorney docket no. 38814-351PC), filed Dec. 19, 2001 and Taiwanese application (attorney docket no. 38814-351TW), filed Dec. 19, 2001. The subject matter of each of these applications is incorporated by reference in its entirety.

### FIELD OF THE INVENTION

The present invention relates to multimeric-protein-complexes, redox proteins, and recombinant polypeptides; and improved methods for their production.

### BACKGROUND

Multimeric proteins (i.e. proteins compising multiple polypeptide chains) are a biologically and commercially important class of proteins. Antibodies for example are multimeric proteins which are used to treat a wide range of disease conditions. However in view of their complexity, multimeric proteins frequently represent significant manufacturing challenges.

Redox proteins are also a commercially important class of proteins with applications in a variety of different industries including the pharmaceutical, personal care and food industry. For example, the redox protein thioredoxin may be used in the manufacture of personal care products (Japanese Patent Applications JP9012471A2, JP103743A2, JP1129785A2), pharmaceutical compositions/products (Aota et al. (1996) J. Cardiov. Pharmacol. (1996) 27: 727-732) as well as to reduce protein allergens present in food products such as milk (del Val et al. (1999) J. Allerg. Vlin. Immunol. 103: 690-697) and wheat (Buchanan et al. (1997) Proc. Natl. Acad. Sci. USA 94: 5372-5377).

However, there is a need in the art to further improve the methods for the recombinant expression of multimeric proteins, including redox proteins. The present invention satifies this need and provides related advantages as well.

### SUMMARY OF THE INVENTION

The present invention relates to novel and improved methods of producing a first and/or second recombinant polypeptides, multimeric-protein-complexes, heteromultimeric-protein-complexes, multimeric-fusion-proteins, heteromultimeric-fusion-proteins, immunoglobulin-polypeptide-chains, immunoglobulins, redox-fusion-polypeptides, and/or thioredoxin-related proteins; in association with oil bodies. Accordingly, provided herein are methods of producing a recombinant multimeric-protein-complex, said method comprising: (a) producing in a cell comprising oil bodies, a first recombinant polypeptide and a second recombinant polypeptide wherein said first recombinant polypeptide is capable of associating with said second recombinant polypeptide to form said multimeric-protein-complex; and (b) associating said multimeric-protein-complex with an oil body through an oil-body-targeting-protein capable of associating with said oil bodies and said first recombinant polypeptide.

The method further contemplates isolating the oil bodies associated with said recombinant multimeric-protein-complex. The second recombinant polypeptide can be associated with a second oil-body-targeting-protein capable of associating with an oil body and said second recombinant polypeptide. Each of said oil-body-targeting-proteins can be a oil-body-protein or an immunoglobulin. The oil-body-targeting-protein can be an oleosin or caleosin. When the oil-body-targeting-protein can be an oleosin or caleosin, the first recombinant polypeptide can be fused to said oleosin or caleosin. Likewise, the second recombinant polypeptide can be fused to a second oleosin or second caleosin capable of associating with an oil body. The first and second recombinant polypeptides can be produced as a multimereic-fusion-protein comprising said first and second polypetide, and can form a multimeric-protein-complex. The multimeric-protein-complex can be a heteromultimeric-protein-complex, and the heteromultimeric-protein-complex can be an enzymatically active redox complex or an immunoglobulin. In one embodiment, the first recombinant polypeptide is capable of associating with said second recombinant polypeptide in the cell. In another embodiment, the first recombinant polypeptide can be a thioredoxin and the second recombinant polypeptide can be a thioredoxin-reductase. In particular embodiments, the thioredoxin can be selected from the group consisting of SEQ ID NOs:38, 42, 46, 50 and SEQ ID NOs:52-194; and the thioredoxin-reductase can be selected from the group consisting of those set forth in SEQ ID NOs:8, 9, 10, 40, 44, 48, 50 and SEQ ID NOs:195-313. In another embodiment, the first recombinant polypeptide can be an immunoglobulin-polypeptide-chain. For example, the first recombinant polypeptide can be an immunoglobulin light chain, or an immunologically active portion thereof, and the second recombinant polypeptide can be an immunoglobulin heavy chain, or an immunologically active portion thereof. In this embodiment, the oil-body-targeting-protein can comprise protein A, protein L or protein G. The cell can be a plant cell, such as a safflower cell, and the like.

Also provided herein is a method of expressing a recombinant multimeric-protein-complex comprising a first and second recombinant polypeptide in a cell, said method comprising:

(a) introducing into a cell a first chimeric nucleic acid sequence comprising:

(i) a first nucleic acid sequence capable of regulating transcription in said cell operatively linked to;

(ii) a second nucleic acid sequence encoding a first recombinant polypeptide;

(b) introducing into said cell a second chimeric nucleic acid sequence comprising:

(i) a third nucleic acid sequence capable of regulating transcription in said cell operatively linked to;

(ii) a fourth nucleic acid sequence encoding a second recombinant polypeptide;

(c) growing said cell under conditions to permit expression of said first and second recombinant polypeptide in a progeny cell comprising oil bodies wherein said first recombinant polypeptide and said second recombinant polypeptide are capable of forming a multimeric-protein-complex; and

(d) associating said first recombinant polypeptide with an oil body through an oil-body-targeting-protein capable of associating with said oil bodies and said first recombinant polypeptide. This method further contemplates isolating from the progeny cell, oil bodies comprising the multimeric-protein-complex. The second recombinant polypeptide can be associated with a second oil-body-targeting-protein capable of associating with an oil body and second recombinant polypeptide. Each of said oil-body-targeting-proteins can be a oil-body-protein or an immunoglobulin. The oil-body-targeting-protein can be an oleosin or caleosin. When the oil-body-targeting-protein is an oleosin or caleosin, the first recombinant polypeptide can be fused to said oleosin or caleosin. Likewise, the second recombinant polypeptide can be fused to a second oleosin or second caleosin capable of associating with an oil body. The first and second recombinant polypeptides can be produced as a multimereic-fusion-protein comprising said first and second polypetide, and can form a multimeric-protein-complex. The multimeric-protein-complex can be a heteromultimeric-protein-complex, and the heteromultimeric-protein-complex can be an enzymatically active redox complex or an immunoglobulin. In one embodiment, the first recombinant polypeptide and said second recombinant polypeptide are capable of forming a multimeric-protein-complex in said progeny cell. In another embodiment, the first recombinant polypeptide can be a thioredoxin and the second recombinant polypeptide can be a thioredoxin-reductase. In particular embodiments, the thioredoxin can be selected from the group consisting of SEQ ID NOs:38, 42, 46, 50 and SEQ ID NOs:52-194; and the thioredoxin-reductase can be selected from the group consisting of those set forth in SEQ ID NOs:8, 9, 10, 40, 44, 48, 50 and SEQ ID NOs:195-313. In another embodiment, the first recombinant polypeptide can be an immunoglobulin-polypeptide-chain. For example, the first recombinant polypeptide can be an immunoglobulin light chain, or an immunologically active portion thereof, and the second recombinant polypeptide can be an immunoglobulin heavy chain, or an immunologically active portion thereof. In this embodiment, the oil-body-targeting-protein can comprise protein A, protein L or protein G. The cell can be a plant cell, such as a safflower cell, and the like.

Also provided herein are methods of producing in a plant a recombinant multimeric-protein-complex, said method comprising:

(a) preparing a first plant comprising cells, said cells comprising oil bodies and a first recombinant polypeptide wherein said first recombinant polypeptide is capable of associating with said oil bodies through an oil-body-targeting-protein;

(b) preparing a second plant comprising cells, said cells comprising oil bodies and a second recombinant polypeptide; and

(c) sexually crossing said first plant with said second plant to produce a progeny plant comprising cells, said cells comprising oil bodies, wherein said oil bodies are capable of associating with said first recombinant polypeptide, and said first recombinant recombinant polypeptide is capable of associating with said second recombinant polypeptide to form said recombinant multimeric-protein-complex. The second recombinant polypeptide can be associated with oil bodies through a second oil-body-targeting-protein in the second plant. The oil bodies can be isolated from the progeny plant comprising said multimeric-protein-complex. The oil-body-targeting-protein can be selected from an oil-body-protein or an immunoglobulin, wherein the oil-body-protein can be an oleosin or caleosin. The first recombinant polypeptide can be fused to the oleosin or caleosin; and the second recombinant polypeptide can be fused to a second oleosin or second caleosin capable of associating with an oil body. The first and second recombinant polypeptide can form a multimeric-protein-complex, such as a heteromultimeric-protein-complex, wherein the heteromultimeric-protein-complex can be an enzymatically active redox complex or an immunoglobulin. In a particular embodiment, the first recombinant polypeptide is a thioredoxin and the second recombinant polypeptide is a thioredoxin-reductase. The thioredoxin can be selected from the group consisting of SEQ ID NOs:38, 42, 46, 50 and SEQ ID NOs:52-194; and the thioredoxin-reductase can be selected from the group consisting of those set forth in SEQ ID NOs:8, 9, 10, 40, 44, 48, 50 and SEQ ID NOs:195-313. In another embodiment, the first recombinant polypeptide can be an immunoglobulin-polypeptide-chain. For example, the first recombinant polypeptide can be an immunoglobulin light chain, or an immunologically active portion thereof, and the second recombinant polypeptide can be an immunoglobulin heavy chain, or an immunologically active portion thereof. In this embodiment, the oil-body-targeting-protein can comprise protein A, protein L or protein G. The plant can be a safflower plant.

Also provided herein are chimeric nucleic acids encoding a multimeric-fusion-protein as described herein, said nucleic acid comprising:

(a) a first nucleic acid sequence encoding an oil-body-targeting-protein operatively linked in reading frame to;

(b) a second nucleic acid sequence encoding a first recombinant polypeptide; linked in reading frame to;

(c) a third nucleic acid sequence encoding a second recombinant polypeptide, wherein said first and second recombinant polypeptide are capable of forming a multimeric-protein-complex. The oil-body-targeting-protein can be selected from an oil-body-protein or an immunoglobulin. The oil-body-protein can be an oleosin or caleosin. The multimeric-protein-complex can be a heteromultimeric-protein-complex, and the first and second recombinant polypeptide can form an enzymatically active heteromultimeric redox complex or an immunoglobulin. In a particular embodiment, the first recombinant polypeptide is a thioredoxin and the second recombinant polypeptide is a thioredoxin-reductase. The thioredoxin can be selected from the group consisting of SEQ ID NOs:38, 42, 46, 50 and SEQ ID NOs:52-194; and the thioredoxin-reductase can be selected from the group consisting of those set forth in SEQ ID NOs:8, 9, 10, 40, 44, 48, 50 and SEQ ID NOs:195-313. In another embodiment, the first recombinant polypeptide can be an immunoglobulin-polypeptide-chain. For example, the first recombinant polypeptide can be an immunoglobulin light chain, or an immunologically active portion thereof, and the second recombinant polypeptide can be an immunoglobulin heavy chain, or an immunologically active portion thereof. In this embodiment, the oil-body-targeting-protein can comprise protein A, protein L or protein G. In yet another embodiment, positioned between the nucleic acid sequence encoding an oil-body-targeting-protein and the nucleic acid sequence encoding a first recombinant polypeptide can be a linker nucleic acid sequence encoding a oil-body-surface-avoiding linker amino acid sequence. The oil-body-surface-avoiding linker amino acid sequence can be substantially negatively charged, or have a molecular weight of at least 35 kd. Optionally, the gene fusion further comprises a linker nucleic acid sequence encoding an amino acid sequence that is specifically cleavable by an enzyme or a chemical, wherein the linker sequence is positioned between the oil-body-surface-avoiding linker amino acid sequence that is also a non-proteolytic linker and said sequence encoding the first recombinant polypeptide.

Also provided herein are recombinant multimeric-fusion-proteins comprising (i) an oil-body-targeting-protein, or fragment thereof, (ii) a first recombinant polypeptide and a (iii) second recombinant polypeptide, wherein said first and second recombinant polypeptides are capable of forming a multimeric-protein-complex. The oil-body-targeting-protein can be selected from an oil-body-protein or an immunoglobulin, and the oil-body-protein can be an oleosin or a caleosin. The multimeric-fusion-protein can be a heteromultimeric-fusion-protein, wherein said first and second recombinant polypeptide form an enzymatically active heteromultimeric redox complex or an immunoglobulin. In a particular embodiment, the first recombinant polypeptide is a thioredoxin and the second recombinant polypeptide is a thioredoxin-reductase. The thioredoxin can be selected from the group consisting of SEQ ID NOs:38, 42, 46, 50 and SEQ ID NOs:52-194; and the thioredoxin-reductase can be selected from the group consisting of those set forth in SEQ ID NOs:8, 9, 10, 40, 44, 48, 50 and SEQ ID NOs:195-313. In another embodiment, the first recombinant polypeptide can be an immunoglobulin-polypeptide-chain. For example, the first recombinant polypeptide can be an immunoglobulin light chain, or an immunologically active portion thereof, and the second recombinant polypeptide can be an immunoglobulin heavy chain, or an immunologically active portion thereof. In this embodiment, the oil-body-targeting-protein can comprise protein A, protein L or protein G. In yet another embodiment, positioned between the nucleic acid sequence encoding an oil-body-targeting-protein and the nucleic acid sequence encoding a first recombinant polypeptide can be a linker nucleic acid sequence encoding a oil-body-surface-avoiding linker amino acid sequence. The oil-body-surface-avoiding linker amino acid sequence can be substantially negatively charged, or have a molecular weight of at least 35 kd. Optionally, the gene fusion further comprises a linker nucleic acid sequence encoding an amino acid sequence that is specifically cleavable by an enzyme or a chemical, wherein the linker sequence is positioned between the oil-body-surface-avoiding linker amino acid sequence and said sequence encoding the first recombinant polypeptide.

Also provide herein are isolated oil bodies comprising a multimeric-protein-complex comprising (i) an oil-body-targeting-protein and (ii) a first recombinant polypeptide, said oil bodies further comprising a second recombinant polypeptide, wherein said first and second recombinant polypeptide are capable of forming a multimeric-protein-complex. The oil-body-targeting-protein can be selected from an oil-body-protein or an immunoglobulin, and the oil-body-protein can be an oleosin or a caleosin. The multimeric-fusion-protein can be a heteromultimeric-fusion-protein, wherein said first and second recombinant polypeptide form an enzymatically active heteromultimeric redox complex or an immunoglobulin. In a particular embodiment, the first recombinant polypeptide is a thioredoxin and the second recombinant polypeptide is a thioredoxin-reductase. In another embodiment, the first recombinant polypeptide can be an immunoglobulin-polypeptide-chain. For example, the first recombinant polypeptide can be an immunoglobulin light chain, or an immunologically active portion thereof, and the second recombinant polypeptide can be an immunoglobulin heavy chain, or an immunologically active portion thereof. In this embodiment, the oil-body-targeting-protein can comprise protein A, protein L or protein G.

Also provided herein are isolated oil bodies comprising

(a) a first fusion protein comprising a first oil-body-targeting-protein fused to a first recombinant polypeptide; and

(b) a second fusion protein comprising a second oil-body-targeting-protein fused to a second recombinant polypeptide,

wherein said first and second recombinant polypeptide are capable of forming a multimeric-protein-complex. The oil-body-targeting-protein can be selected from an oil-body-protein or an immunoglobulin, and the oil-body-protein can be an oleosin or a caleosin. The multimeric-fusion-protein can be a heteromultimeric-fusion-protein, wherein said first and second recombinant polypeptide form an enzymatically active heteromultimeric redox complex or an immunoglobulin. In a particular embodiment, the first recombinant polypeptide is a thioredoxin and the second recombinant polypeptide is a thioredoxin-reductase. The thioredoxin can be selected from the group consisting of SEQ ID NOs:38, 42, 46, 50 and SEQ ID NOs:52-194; and the thioredoxin-reductase can be selected from the group consisting of those set forth in SEQ ID NOs:8, 9, 10, 40, 44, 48, 50 and SEQ ID NOs:195-313. In another embodiment, the first recombinant polypeptide can be an immunoglobulin-polypeptide-chain. For example, the first recombinant polypeptide can be an immunoglobulin light chain, or an immunologically active portion thereof, and the second recombinant polypeptide can be an immunoglobulin heavy chain, or an immunologically active portion thereof. In this embodiment, the oil-body-targeting-protein can comprise protein A, protein L or protein G.

Also provided are cells and transgenic plants comprising oil bodies, multimeric-protein-complexes, multimeric-fusion-proteins, set forth herein. In one embodiment, the first recombinant polypeptide can be an immunoglobulin-polypeptide-chain. For example, the first recombinant polypeptide can be an immunoglobulin light chain, or an immunologically active portion thereof, and the second recombinant polypeptide can be an immunoglobulin heavy chain, or an immunologically active portion thereof. In this embodiment, the oil-body-targeting-protein can comprise protein A, protein L or protein G. In embodiments, wherein said first recombinant polypeptide is a thioredoxin and said second recombinant polypeptide is a thioredoxin-reductase, the methods described herein can be used to formulate the oil bodies for use in the preparation of a food product, personal care product or pharmaceutical composition. These formulations can further comprise the addition of NADP or NADPH. The food product can be a milk or wheat based food product. The personal care product can reduce the oxidative stress to the surface area of the human body or can be used to lighten the skin. The pharmaceutical composition can be used to treat chronic obstructive pulmonary disease (COPD), cataracts, diabetes, envenomation, bronchiopulmonary disease, malignancies, psoriasis, reperfusion injury, wound healing, sepsis, GI bleeding, intestinal bowel disease (IBD), ulcers, GERD (gastro esophageal reflux disease).

Also provided herein are compositions comprising isolated oil bodies, thioredoxin and thioredoxin-reductase, wherein said thioredoxin can be selected from the group consisting of SEQ ID NOs:38, 42, 46, 50 and SEQ ID NOs:52-194, and said thioredoxin-reductase can be selected from the group consisting of those set forth in SEQ ID NOs:8, 9, 10, 40, 44, 48, 50 and SEQ ID NOs:195-313. The composition can further comprise NADP or NADPH. In another embodiment, the composition comprises a first recombinant polypeptide that can be an immunoglobulin-polypeptide-chain and a second recombinant polypeptide. For example, the first recombinant polypeptide can be an immunoglobulin light chain, or an immunologically active portion thereof, and the second recombinant polypeptide can be an immunoglobulin heavy chain, or an immunologically active portion thereof. In this embodiment, the oil-body-targeting-protein can comprise protein A, protein L or protein G.

Also provided are multimeric-fusion-proteins, wherein the fusion-protein contains two or more polypeptide chains selected from the group of proteins set forth in FIG. 5. Methods are also provided of reducing allergenicity of a food comprising the steps of providing the isolated oil bodies set forth herein; and adding the isolated oil bodies to the food, whereby allergenicity of the food is reduced. The food can be selected from the group consisting of wheat flour, wheat dough, milk, cheese, yogurt and ice cream. The various methods of treating food can further comprise providing NADH as a co-factor in the substantial absence of NADPH.

Also provided herein are methods of treating or protecting a target against oxidative stress, comprising the steps of providing the recombinant redox fusion polypeptide comprising thioredoxin and thioredoxin-reductase; and contacting the recombinant fusion polypeptide with a target, wherein the target is susceptible to oxidative stress, thereby treating or protecting against the stress. The target can be selected from the group consisting of a molecule, a molecular complex, a cell, a tissue, and an organ.

Also provided herein are methods for preparing an enzymatically active redox protein associated with oil bodies comprising:

a) producing in a cell a redox fusion polypeptide comprising a first redox protein linked to a second redox protein;

b) associating said redox fusion polypeptide with oil bodies through an oil-body-targeting-protein capable of associating with said redox fusion polypeptide and said oil bodies; and

c) isolating said oil bodies associated with said redox fusion polypeptide. The first redox protein can be a thioredoxin and the second redox protein can be a thioredoxin-reductase.

Also, provided herein are methods of producing an immunoglobulin, said method comprising: (a) producing in a cell comprising oil bodies, a first immunoglobulin-polypeptide-chain and a second immunoglobulin-polypeptide-chain wherein said first immunoglobulin-polypeptide-chain is capable of associating with said second immunoglobulin-polypeptide-chain to form said immunoglobulin; and (b) associating said immunoglobulin with an oil body through an oil-body-targeting-protein capable of associating with said oil bodies and said first immunoglobulin-polypeptide-chain. For example, the first immunoglobulin-polypeptide-chain can be an immunoglobulin light chain, or an immunologically active portion thereof, and the second immunoglobulin-polypeptide-chain can be an immunoglobulin heavy chain, or an immunologically active portion thereof. In this embodiment, the oil-body-targeting-protein can comprise protein A, protein L or protein G.

Also provided herein are methods for preparing a redox protein or an immunoglobulin associated with oil bodies comprising:

a) introducing into a cell a chimeric nucleic acid sequence comprising:

1) a first nucleic acid sequence capable of regulating transcription in said cell operatively linked to;

2) a second nucleic acid sequence encoding a recombinant fusion polypeptide comprising (i) a nucleic acid sequence encoding a sufficient portion of an oil-body-protein to provide targeting of said recombinant fusion polypeptide to an oil body linked to (ii) a nucleic acid sequence encoding a redox fusion polypeptide comprising a first redox protein linked to a second redox protein, or a nucleic acid sequence encoding a immunoglobulin comprising a first immunoglobulin-polypeptide-chain linked to a second immunoglobulin-polypeptide-chain, operatively linked to;

3) a third nucleic acid sequence capable of terminating transcription in said cell;

b) growing said cell under conditions to permit expression of said redox fusion polypeptide or immunoglobulin in a progeny cell comprising oil bodies; and

c) isolating from said progeny cell said oil bodies comprising said redox fusion polypeptide or immunoglobulin. In certain embodiments, positioned between said nucleic acid sequence encoding a sufficient portion of an oil-body-protein and said nucleic acid sequence encoding a redox fusion polypeptide or immunoglobulin can be a linker nucleic acid sequence encoding a oil-body-surface-avoiding linker amino acid sequence. The oil-body-surface-avoiding linker amino acid sequence can be substantially negatively charged or have a molecular weight of at least 35 kd. Optionally, the gene fusion further comprises a linker nucleic acid sequence encoding an amino acid sequence that is specifically cleavable by an enzyme or a chemical, wherein the linker sequence is positioned between the oil-body-surface-avoiding linker amino acid sequence and said nucleic acid sequence encoding a redox fusion polypeptide. In this optional embodiment, also contemplated is the introduction of an enzyme or chemical that cleaves said redox fusion polypeptide from said oil body, thereby obtaining isolated redox fusion polypeptide. The first redox protein can be a thioredoxin and said second redox protein can be a thioredoxin-reductase. In one embodiment, the thioredoxin and thioredoxin-reductase can be obtained from Arabidopsis. In another embodiment, the first redox protein is at least 5 times more active when produced as a redox fusion polypeptide as compared to the production of the first redox protein without the second redox protein.

Also provided herein, for use with the various methods set forth herein is the formulation of an emulsion of the oil bodies associated with the redox fusion polypeptide for use in the preparation of a product capable of treating oxidative stress in a target, a product capable of chemically reducing a target, pharmaceutical composition, a personal care product or a food product. Accordingly, an emulsion formulation composition is provided.

Also provided herein is a chimeric nucleic acid comprising:

1) a first nucleic acid sequence capable of regulating transcription in a host cell operatively linked to;

2) a second nucleic acid sequence encoding a recombinant fusion polypeptide comprising (i) a nucleic acid sequence encoding a sufficient portion of an oil-body-protein to provide targeting of said recombinant fusion polypeptide to an oil body linked to (ii) a nucleic acid sequence encoding a redox fusion polypeptide comprising a first redox protein linked to a second redox protein operatively linked to;

3) a third nucleic acid sequence capable of terminating transcription in said cell. The oil-body-protein can be an oleosin or a caleosin, the first redox protein can be a thioredoxin and said second redox protein can be a thioredoxin-reductase. In certain embodiments, positioned between said nucleic acid sequence encoding a sufficient portion of an oil-body-protein and said nucleic acid sequence encoding a redox fusion polypeptide is a linker nucleic acid sequence encoding a oil-body-surface-avoiding linker amino acid sequence. The oil-body-surface-avoiding linker amino acid sequence can be substantially negatively charged, or have a molecular weight of at least 35 kd. In one embodiment, the gene fusion optionally further comprises a linker nucleic acid sequence encoding an amino acid sequence that is specifically cleavable by an enzyme or a chemical, wherein the linker sequence is positioned between the oil-body-surface-avoiding linker amino acid sequence and said nucleic acid sequence encoding a redox fusion polypeptide.

Also provided herein are transgenenic plants, e.g., safflower plants, comprising any of the chimeric nucleic acid sequences and constructs described herein. The chimeric nucleic acids can be contained within a plastid. Accordingly, isolated plastids are provided having chimeric nucleic acids therein. Also provided are plant seeds comprising the chimeric nucleic acids provided herein.

Also provided are oil body preparations obtained using any of the methods provided herein, and food products, pharmaceutical compositions, and personal care products containing the oil body preparations. The products and/or compositions provided herein are capable of treating oxidative stress in a target, capable of chemically reducing a target. Also provided is a deteregent composition comprising an oil body preparation capable of chemically reducing a target, and related methods of cleansing an item, comprising administering such product to the item under conditions that promote cleansing.

Also provided herein are nucleic acid constructs comprising a gene fusion, wherein the gene fusion comprises a first region encoding an oil-body-protein or an active fragment thereof, operably linked to a second region encoding at least one thioredoxin-related protein or an active fragment thereof. In one embodiment, the at least one thioredoxin-related protein can be thioredoxin. The thioredoxin can be selected from the group consisting of SEQ ID NOs:38, 42, 46, 50 and SEQ ID NOs:52-194. The thioredoxin can be obtained from Arabidopsis or wheat.

In another embodiment, the at least one thioredoxin-related protein can be thioredoxin-reductase. The thioredoxin-reductase can selected from the group consisting of those set forth in SEQ ID NOs:8, 9, 10, 40, 44, 48, 50 and SEQ ID NOs:195-313 and/or derived from Arabidopsis or wheat. The thioredoxin-reductase can be an NADPH-dependent thioredoxin-reductase. The second region can encode a thioredoxin and thioredoxin-reductase. In one embodiment, the thioredoxin and thioredoxin-reductase is obtained from Mycobacterium leprae. In another embodiment, the at least one thioredoxin-related protein can be an engineered fusion protein. The first region can precede, in a 5′ to 3′ direction, the second region. Alternatively, the first region follows, in a 5′ to 3′ direction, the second region. The gene fusion can optionally further comprise a third region encoding a second thioredoxin-related protein or an active fragment thereof, operably linked to the first region, or to the second region, or to both. A seed-specific promoter, such as a phaseolin promoter, can be operably linked to the gene fusion. In one embodiment, at least one thioredoxin-related protein is derived from a plant species selected from the group consisting of Arabidopsis and wheat. In another embodiment, at least one thioreoxin-related protein can be derived from E. coli.

In one embodiment, the gene fusion further comprises a nucleic acid sequence encoding a oil-body-surface-avoiding linker amino acid sequence, wherein the linker amino acid sequence is positioned between the first region and the second region. The oil-body-surface-avoiding linker amino acid sequence can be substantially negatively charged, or have a molecular weight of at least 35 kd. In addition, the gene fusion can further comprise a linker nucleic acid sequence encoding an amino acid sequence that is specifically cleavable by an enzyme or a chemical, wherein the linker sequence is positioned between the oil-body-surface-avoiding linker amino acid sequence and the second region.

Also provided herein are transgenic plants containing a nucleic acid construct comprising a gene fusion, wherein the gene fusion comprises a region encoding an oil-body-protein or an active fragment thereof, operably linked to a region encoding a first thioredoxin-related protein or an active fragment thereof. The thioredoxin-related protein can be thioredoxin. The nucleic acid construct can be contained within a plastid. In one embodiment, when the first thioredoxin-related protein is thioredoxin and the construct can further comprise a region encoding a thioredoxin-reductase. The gene fusion can optionally further comprise a third region encoding a second thioredoxin-related protein or an active fragment thereof, operably linked to the first region, or to the second region, or to both. The gene fusion can further optionally further comprise a nucleic acid sequence encoding a oil-body-surface-avoiding linker amino acid sequence, wherein the nucleic acid encoding the linker amino acid sequence is positioned between the region encoding an oil-body-protein and the region encoding a first thioredoxin-related protein. The oil-body-surface-avoiding linker amino acid sequence can be substantially negatively charged, or have a molecular weight of at least 35 kd. The gene fusion can optionally further comprise a linker nucleic acid sequence encoding an amino acid sequence that is specifically cleavable by an enzyme or a chemical, wherein the linker sequence is positioned between the oil-body-surface-avoiding linker amino acid sequence and the region encoding a first thioredoxin-related protein.

Also provided is a transgenic plant comprising a nucleic acid construct, a seed-specific promoter operably linked to a gene fusion, wherein the gene fusion comprises a region encoding an oil-body-protein or an active fragment thereof, operably linked to a region encoding a first thioredoxin-related protein or an active fragment thereof, wherein a fusion protein comprising activities of oleosin and the thioredoxin-related protein is produced in a seed of the plant. In another embodiment, a thioredoxin-related protein having concentration of at least about 0.5% of total cellular seed protein is provided. Also provided herein is an extract comprising an activity of a thioredoxin-related protein. Also provided are oil bodies and/or oil obtained from various seeds.

Also provided herein are methods of making a fusion protein comprising a thioredoxin-related activity, the method comprising the steps of:

a) providing a transgenic plant comprising a nucleic acid construct comprising a seed-specific promoter operably linked to a gene fusion, wherein the gene fusion comprises a region encoding an oil-body-protein or an active fragment thereof, operably linked to a region encoding a first thioredoxin-related protein or an active fragment thereof, the gene fusion encoding a fusion protein comprising a thioredoxin-related activity;

b) obtaining seeds from the plant; and

c) recovering the fusion protein by isolating oil bodies from the seeds. In one embodiment, the oil bodies are fractionated to achieve partial purification of the fusion protein. The oil bodies can be in association with a fusion protein. The oil-body-protein can be cleaved from the thioredoxin-related protein after fractionation of the oil bodies. The cleaving step can make use of a protease or chemical proteolysis.

Also provided herein are methods of reducing allergenicity of a food comprising the steps of:

a) providing a preparation comprising oil bodies associated with a fusion protein, the fusion protein comprising an oil-body-protein or an active fragment thereof and a thioredoxin-related protein or an active fragment thereof; and

b) adding the preparation to the food, whereby allergenicity of the food is reduced due to activity of the thioredoxin-related protein or fragment. The food can be wheat flour, wheat dough, milk, cheese, yogurt and ice cream. In one embodiment, NADH is used as a co-factor in the substantial absence of NADPH.

Also provided herein are pharmaceutical compositions comprising a fusion protein, the fusion protein comprising an oil-body-protein or an active fragment thereof and a thioredoxin-related protein or an active fragment thereof, in a pharmaceutically acceptable carrier. The oil bodies can be associated with the fusion protein. Also provided is a cosmetic formulation comprising oil bodies associated with a fusion protein, the fusion protein comprising an oil-body-protein or an active fragment thereof and a thioredoxin-related protein or an active fragment thereof, in a pharmaceutically acceptable carrier. Also provided are methods of treating or protecting a target against oxidative stress, comprising the steps of:

a) providing a preparation comprising a fusion protein, the fusion protein comprising an oil-body-protein or an active fragment thereof and a thioredoxin-related protein or an active fragment thereof; and

b) contacting the preparation with a target, wherein the target is susceptible to oxidative stress, thereby treating or protecting against the stress. The target can be selected from the group consisting of a molecule, a molecular complex, a cell, a tissue, and an organ.

Also provided is a nucleic acid construct comprising a gene fusion, wherein the gene fusion comprises a first region encoding an oil-body-protein or an active fragment thereof, operably linked to a second region encoding at least one polypeptide or an active fragment thereof, and an oil-body-surface-avoiding linker in frame between the first and second region polypeptides. Also provided are methods of expressing this construct into the encoded amino acid sequence; and oil bodies, formulations, emulsions, cells, and plants comprising the construct and encoded amino acid sequence. These particular constructs, oil bodies, formulations, emulsions, cells, and plants can be produced according to the methods described herein. The second region can encode any polypeptied, for example, a therapeutically, nutritionally, industrially or cosmetically useful peptide as set forth herein. For example, the second region can encode a redox protein, an immunoglobulin, a thioredoxin-related protein or any one or more recombinant polypeptides of a multimeric-protein-complex.

Other features and advantages of the present invention will become readily apparent from the following detailed description. It should be understood however that the detailed description and the specific examples while indicating particular embodiments of the invention are given by way of illustration only.

### BRIEF DESCRIPTION OF THE DRAWINGS

FIG. 1 shows a ClustalW Formatted Alignment comparison of the published NADPH thioredoxin-reductase nucleic acid sequence (SEQ ID NO:9) (ATTHIREDB-Jacquot et al. J. Mol. Biol. (1994) 235 (4):1357-63.) with the sequence isolated herein in Example 1 (TR; SEQ ID NO:8).

FIG. 2 shows a ClustalW Formatted Alignment comparison of the deduced amino acid sequence of the published NADPH thioredoxin-reductase sequence (SEQ ID NO:12)(ATTHIREDB Jacquot et al. J. Mol. Biol. (1994) 235 (4):1357-63.) with the sequence isolated herein in Example 1 (TR; SEQ ID NO:13).

FIG. 3 shows a clustal alignment comparing the amino acid sequence of the Arabidopsis thaliana thioredoxin-reductase-linker-thioredoxin synthetic fusion (Arab TR-link-Trxh; SEQ ID NO:37) to the Mycobacterium leprae thioredoxin-reductase-thioredoxin natural fusion (M.lep TR/Trxh; SEQ ID NO:36) natural fusion. Overall, the proteins are approximately 50% identical at the amino acid level.

FIG. 4 is a bar graph showing the thioredoxin/thioredoxin-reductase activity measurements for the various transgenic Arabidopsis seed fractions. Relative specific activity is expressed as a percentage of the E. coli thioredoxin and thioredoxin-reductase activities. The numbered bars in the graph correspond to the following:

1. W.T.+oleosin-thioredoxin

2. W.T.+thioredoxin-oleosin

3. W.T.+thioredoxin

4. W.T.+oleosin-thioredoxin-reductase

5. W.T.+thioredoxin-reductase-oleosin

6. W.T.+thioredoxin-reductase

7. thioredoxin+oleosin-thioredoxin-reductase

8. thioredoxin+thioredoxin-reductase-oleosin

9. thioredoxin+thioredoxin-reductase

10. thioredoxin-reductase+oleosin-thioredoxin

11. thioredoxin-reductase+thioredoxin-oleosin

12. oleosin-M.lep TR/Trxh

13. E. coli thioredoxin-reductase+thioredoxin

FIG. 5 provides a listing of exemplary proteins for use in the heteromultimeric-fusion-proteins and heteromultimeric-protein-complexes provided herein.

### DETAILED DESCRIPTION

As hereinbefore mentioned, the present invention relates to novel and improved methods for the production of multimeric proteins, including a first and second recombinant polypeptide, multimeric-protein-complexes, heteromultimeric-protein-complexes, multimeric-fusion-proteins, heteromultimeric-fusion-proteins, immunoglobulin-polypeptide-chains, immunoglobulins, redox-fusion-polypeptides, and a first and second thioredoxin-related protein; and related products. These methods permit the production of active multimeric-protein-complexes in association with oil bodies. The oil bodies in association with the multimeric-protein-complex may be used to prepare various useful emulsions.

Accordingly, provided herein are methods of producing a recombinant multimeric-protein-complex associated with an oil body, said method comprising:

(a) producing in a cell comprising oil bodies, a first recombinant polypeptide and a second recombinant polypeptide wherein said first recombinant polypeptide is capable of associating with said second recombinant polypeptide in the cell to form said multimeric-protein-complex; and

(b) associating said multimeric-protein-complex with an oil body through an oil-body-targeting-protein capable of associating with said oil body and said first recombinant polypeptide.

Definitions and Terms

Unless defined otherwise, all technical and scientific terms used herein have the same meaning as is commonly understood by one of skill in the art to which this invention belongs. Where permitted, all patents, applications, published applications and other publications and sequences from GenBank, SwissPro and other data bases referred to throughout in the disclosure herein are incorporated by reference in their entirety.

As used herein, the phrase “multimeric-protein-complex”, refers to two or more polypeptide chains that permanently or repeatedly interact or permanently or repeatedly coordinate to form a biologically active assembly comprising said two or more polypeptide chains. It should be noted that the polypeptides may be independently biologically active without interaction or coordination to form the complex. The multimeric-protein-complex may provide a biological structure, or it may be capable of facilitating a chemical or biological reaction. For example, one of the protein regions within the multimeric-protein-complex can repeatedly activate or repeatedly inactivate the biological or metabolic activity of one or more of the other proteins contained within the multimeric-protein-complex. In one embodiment, the first and second recombinant polypeptide contained in a multimeric-protein-complex may either associate or interact as independent non-contiguous polypeptide chains or the multimeric-protein-complex may be prepared as a fusion polypeptide (multimeric-fusion-protein) between the first and second recombinant polypeptide.

One example of a repeated (e.g., reoccurring) interaction or association between the two or more polypeptides of a multimeric-protein-complex provided herein is the interaction between two or more non-identical redox proteins to form a heteromultimeric-protein-complex. Exemplary redox proteins for use in this regard are thioredoxin and the thioredoxin-reductase. A further example is the interaction between two or more immunoglobulin-polypeptide-chains to form an immunoglobulin. As used herein, the phrase “heteromultimeric-protein-complex”, refers to two or more non-identical polypeptide chains that permanently or repeatedly interact or permanently or repeatedly coordinate to form a biologically active assembly comprising said two or more polypeptide chains. Other examples of multimeric-protein-complexes provided herein include a first and second recombinant polypeptide, heteromultimeric-protein-complexes, multimeric-fusion-proteins, heteromultimeric-fusion-proteins, immunoglobulins, first and second immunoglobulin-polypeptide-chains, redox-fusion-polypeptides, and a first and second thioredoxin-related protein.

The recombinant polypeptide or multimeric-protein-complex is associated with an oil body. As used herein, the phrase “oil body” or “oil bodies” refers to any oil or fat storage organelle in any cell type. Accordingly, the oil bodies may be obtained from any cell comprising oil bodies, including plant cells (described in for example: Huang (1992) Ann. Rev. Plant Mol. Biol. 43: 177-200), animal cells (described in for example: Murphy (1990) Prog Lipid Res 29(4): 299-324), including adipocytes, hepatocytes, steroigogenic cells, mammary epithelial cells, macrophages, algae cells (described in for example: Rossler (1988) J. Physiol. London, 24: 394-400) fungal cells, including yeast cells (described in for example Leber et al. (1994) Yeast 10: 1421-1428) and bacterial cells (described in for example: Pieper-Furst et al. (1994) J. Bacteriol. 176: 4328-4337). Generally the oil bodies used herein are oil bodies obtainable from plant cells and generally the oil bodies obtainable from plant seed cells.

As used herein, the phrase “is capable of associating with”, “associate” or grammatical variations thereof, refers to any interaction between two or more polypeptides, including any covalent interactions (e.g. multimeric-fusion-proteins) as well as non-covalent interactions. Exemplary non-covalent interactions can be between the oil-body-targeting-protein and a redox protein or immunoglobulin-polypeptide-chain, as well as between two or more different proteins contained within two or more separate oil-body-protein fusion proteins (e.g., the redox proteins in oleosin-thioredoxin and oleosin-thioredoxin-reductase).

As used herein, the term “recombinant” (also referred to as heterologous) in the context of recombinant proteins and amino acids, means “of different natural origin” or represents a non-natural state. For example, if a host cell is transformed with a nucleotide sequence derived from another organism, particularly from another species, that nucleotide sequence and amino acid sequence encoded thereby, is recombinant (heterologous) with respect to that host cell and also with respect to descendants of the host cell which carry that gene. Similarly, recombinant (or heterologous) refers to a nucleotide sequence derived from and inserted into the same natural, original cell type, but which is present in a non-natural state, e.g., a different copy number, or under the control of different regulatory elements. A transforming nucleotide sequence may include a recombinant coding sequence, or recombinant regulatory elements. Alternatively, the transforming nucleotide sequence may be completely heterologous or may include any possible combination of heterologous and endogenous nucleic acid sequences.

In various embodiments of the present invention, the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, and/or thioredoxin-related proteins, are produced in a cell comprising oil bodies. As used herein the phrase “in a cell”, “in the cell”, or grammatical variations thereof, mean that the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, and/or thioredoxin-related proteins, may be produced in any cellular compartment of that cell, so long as that cell comprises oil bodies therein. In embodiments of the invention in which plant cells are used, the phrase is intended to include the plant apoplast.

In various embodiments provided herein, the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, and thioredoxin-related proteins, associate with an oil body through an oil-body-targeting-protein. As used herein, the phrase “oil-body-targeting-protein” refers to any protein, protein fragment or peptide capable of associating with an oil body. Exemplary oil-body-targeting-proteins for use herein include oil-body-proteins, such as oleosin and caleosin; immunoglobulins, such as bi-specific antibodies; and the like.

In embodiments described herein in which an oil-body-protein is used, the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, and thioredoxin-related proteins, are generally fused to the oil-body-protein. The term “oil-body-protein” refers to any protein naturally present in cells and having the capability of association with oil bodies, including any oleosin or caleosin.

Accordingly, provided herein a method of expressing a recombinant multimeric-protein-complex comprising a first and second recombinant polypeptide in a cell, said method comprising:

(a) introducing into a cell a first chimeric nucleic acid sequence comprising:

(i) a first nucleic acid sequence capable of regulating transcription in said cell operatively linked to;

(ii) a second nucleic acid sequence encoding a first recombinant polypeptide, such as a redox protein, an immunoglobulin-polypeptide-chain or an thioredoxin-related protein, fused to an oil-body-protein;

(b) introducing into said cell a second chimeric nucleic acid sequence comprising:

(i) a third nucleic acid sequence capable of regulating transcription in said cell operatively linked to;

(ii) a fourth nucleic acid sequence encoding a second recombinant polypeptide, such as a second redox protein, a second immunoglobulin-polypeptide-chain or a second thioredoxin-related protein,;

(c) growing said cell under conditions to permit expression of said first and second recombinant polypeptide in a progeny cell comprising oil bodies wherein said first recombinant polypeptide and said second recombinant polypeptide are capable of forming a multimeric-protein-complex, preferably in said progeny cell; and

(d) associating said first recombinant polypeptide with an oil body through said oil-body-protein.

The term “nucleic acid” as used herein refers to a sequence of nucleotide or nucleoside monomers consisting of naturally occurring bases, sugars and intersugar (backbone) linkages. The term also includes modified or substituted sequences comprising non-naturally occurring monomers or portions thereof, which function similarly. The nucleic acid sequences may be ribonucleic acids (RNA) or deoxyribonucleic acids (DNA) and may contain naturally occurring bases including adenine, guanine, cytosine, thymidine and uracil. The sequences also may contain modified bases such as xanthine, hypoxanthine, 2-aminoadenine, 6-methyl, 2-propyl and other alkyl adenines, 5-halo-uracil, 5-halo cytosine, 6-aza uracil, 6-aza cytosine and 6-aza thymine, pseudo uracil, 4-thiouracil, 8-halo adenine, 8-amino adenine, 8-thiol-adenine, 8-thio-alkyl adenines, 8-hydroxyl adenine and other 8-substituted adenines, 8-halo guanines, 8 amino guanine, 8 thiol guanine, 8-thioalkyl guanines, 8 hydroxyl guanine and other 8-substituted guanines, other aza and deaza uracils, thymidines, cytosines, adenines, or guanines, 5-trifluoromethyl uracil and 5-trifluoro cytosine.

Multmeric-Protein-Complexes

In accordance with the methods and compositions provided herein, any two recombinant polypeptides capable of forming a multimeric-protein-complex may be used. The nucleic acid sequences encoding the two recombinant polypeptides may be obtained from any biological source or may be prepared synthetically. In general nucleic acid sequence encoding multimeric proteins are known to the art and readily available. Known nucleic acid sequences encoding multimeric-protein-complexes may be used to design and construct nucleic acid sequence based probes in order to uncover and identify previously undiscovered nucleic acid sequences encoding multimeric-protein-complexes, for example, by screening cDNA or genomic libraries or using 2- or multi-hybrid systems. Thus, additional nucleic acid sequences encoding multimeric-protein-complexes may be discovered and used as described herein.

The first and/or second recombinant polypeptides that are comprised within a multimeric-protein-complex provided herein, can themselves be in the form of heteromultimeric-protein-complexes, multimeric-fusion-proteins, heteromultimeric-fusion-proteins, immunoglobulins, immunoglobulin-polypeptide-chains, redox-fusion-polypeptides, and/or a first and/or second thioredoxin-related protein.

The nucleic acid sequence encoding the first and second recombinant polypeptide, heteromultimeric-protein-complexes, multimeric-fusion-proteins, heteromultimeric-fusion-proteins, immunoglobulins, immunoglobulin-polypeptide-chains, redox-fusion-polypeptides, and/or a first and/or second thioredoxin-related protein may be obtained from separate sources or may be obtained from the same source. In general however, such nucleic acid sequence is obtained from the same or a similar biological source. In certain embodiments wherein the nucleic acid sequence encoding the first and second recombinant polypeptide protein are obtained from the same source, the nucleic acid sequence encoding the first recombinant polypeptide and second recombinant polypeptide may be naturally fused. In accordance with a particular embodiment, the nucleic acid sequences encoding the first and second recombinant polypeptide are obtained from a plant source.

Oil-Body-Surface-Avoiding Linkers

Polypeptide spacers or linkers of variable length and/or negative charge can be used herein to separate the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, and the first and/or second thioredoxin-related proteins from the in-frame oil-body-targeting-protein, to improve activity of and/or the accessibility of the polypeptide or complex. For example, in one embodiment set forth herein, positioned between a nucleic acid sequence encoding a sufficient portion of an oil-body-protein and a nucleic acid sequence encoding either the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, and the first and/or second thioredoxin-related proteins; is a linker nucleic acid sequence encoding an oil-body-surface-avoiding linker amino acid sequence.

Oil-body-surface-avoiding linkers are positioned between the oil-body targeting sequence and an in-frame recombinant polypeptide of interest, e.g., the multimeric-protein-complexes provided herein, serve to increase the distance and or decrease the interaction between the negatively charged oil body surface and the recombinant polypeptide of interest. A negatively charged linker is repelled by the negatively charged oil body surface, in turn increasing the distance or decreasing the interaction of its attached recombinant polypeptide with the oil body surface. As a consequence of the increased distance from the oil body surface, the recombinant polypeptide will be more accessible, e.g. to its target(s) substrate, protein substrate, protein partner, and less affected by the charged oil body surface. Exemplary linker sequences for use herein can be either a negatively charged linker, or a linker having a molecular weight of at least about 35 kd or more.

As used herein, a “negatively charged linker” sequence, refers to any amino acid segment, or nucleic acid encoding such, that has a pl less than or equal to the pl of an oil body. In certain embodiments, the pl of the negatively charged linker is about 90%, 80%, 70%, 60%, 50%, 40%, 30%, down to about 25% or more, below that of the pl of an oil body in the particular plant or cell system being used. Exemplary negatively charged linkers can be prepared comprising any combination of the negatively charged amino acid residues. For example, in one embodiment, a negatively charged linker comprises either a poly-glutamate or poly-aspartate sequence, or any combination of both amino acid residues. The negatively charged linker is typically at least 5, 10, 15, 20, 25, 30, 35, 40, 45, 50, 55, 60, 65, 70, 75, 80, 85, 90, 95, 100 or more amino acids in length. The negatively charged linkers are preferably non-proteolytic (e.g., non-proteolytic linkers), having no site for efficient proteolysis. When linker size rather than charge is used to minimize interaction of the recombinant polypeptide of interest with the oil body surface, then the linker is non-proteolytic and ranges in molecular weight from about 35 kd up to about 100 kd. The upper size limit is chosen such that the expression of, the activity of, the conformation of, and/or the access to target of, the recombinant polypeptide of interest is not significantly affected by the linker.

In certain embodiments, described herein where a non-proteolytic linker amino acid sequence is employed, the gene fusion or protein fusion (multimeric-fusion-protein) can optionally further comprise a linker nucleic or amino acid sequence encoding a sequence that is specifically cleavable by an enzyme or a chemical, wherein the linker sequence is positioned between the non-proteolytic linker sequence and sequence encoding the desired recombinant protein region, e.g., the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, or the first and/or second thioredoxin-related proteins set forth herein. When a cleavable linker sequence is used herein, in a particular embodiment, it is further downstream than the non-proteolytic linker sequence from the oil-body-targeting-protein region of the fusion protein. By virtue of cleavable linker, the recombinant fusion polypeptides provided herein, such as the multimeric-fusion-proteins and redox fusion polypeptides, can be isolated and purified by introducing an enzyme or chemical that cleaves said multimeric-fusion-protein and/or redox fusion polypeptide from said oil body, thereby obtaining and/or isolating the desired protein. It is contemplated herein that the use of cleavable linker sequence downstream of the non-proteolytic linker/spacer sequence will improve the yield of protein recovery when isolating or purifying proteins using the methods provided herein.

The nucleic acid sequences encoding the first or second recombinant polypeptide may be altered to improve expression levels for example, by optimizing the nucleic acids sequence in accordance with the preferred codon usage for the particular cell type which is selected for expression of the first and second recombinant polypeptide, or by altering of motifs known to destabilize mRNAs (see for example: PCT Patent Application 97/02352). Comparison of the codon usage of the first and second recombinant polypeptide with codon usage of the host will enable the identification of codons that may be changed. For example, typically plant evolution has tended towards a preference for CG rich nucleotide sequences while bacterial evolution has resulted in bias towards AT rich nucleotide sequences. By modifying the nucleic acid sequences to incorporate nucleic acid sequences preferred by the host cell, expression may be optimized. Construction of synthetic genes by altering codon usage is described in for example PCT patent Application 93/07278. The first and second recombinant polypeptide can be altered using for example targeted mutagenesis, random mutagenesis (Shiraishi et al. (1998) Arch. Biochem. Biophys. 358: 104-115; Galkin et al. (1997) Protein Eng. 10: 687-690; Carugo et al. (1997) Proteins 28: 10-28; Hurley et al. (1996) Biochemistry 35: 5670-5678), gene shuffling, and/or by the addition of organic solvent (Holmberg et al. (1999) Protein Eng. 12: 851-856). Any polypeptide spacers that are used in accordance with the methods and products provided herein may be altered in similar ways.

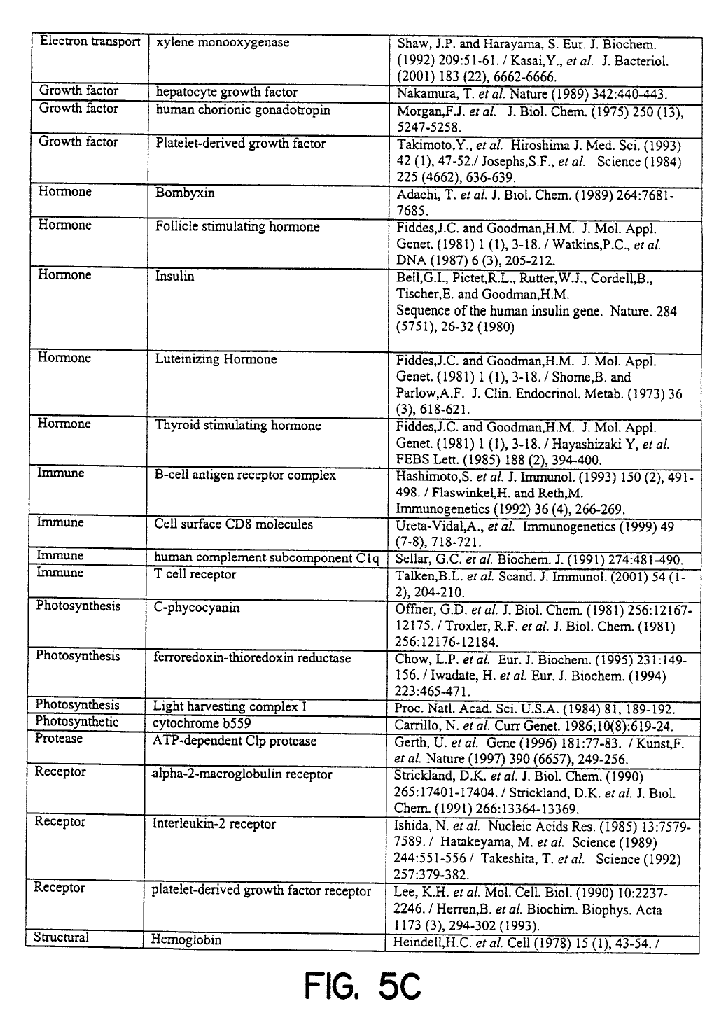

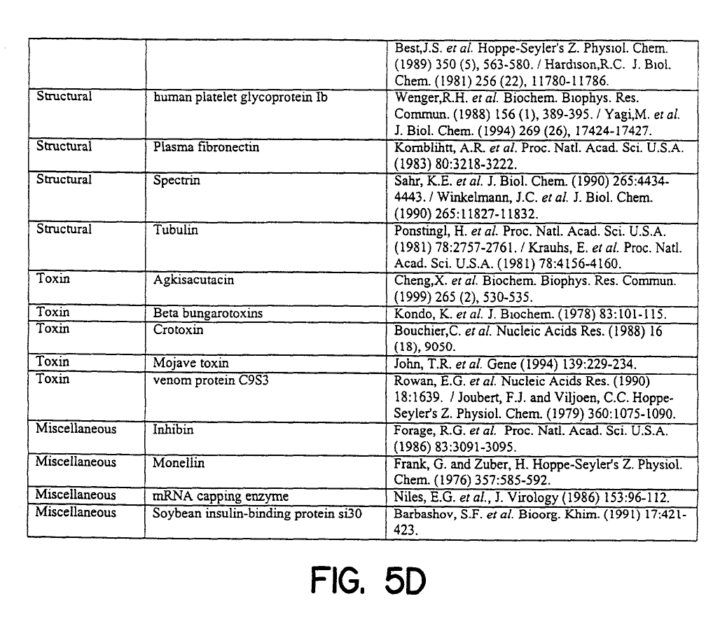

In particular embodiments provided herein, the recombinant polypeptides or thioredoxin-related proteins capable of forming a multimeric-protein-complex are capable of forming a heteromultimeric-protein-complex. Examples of heteromultimeric-protein-complexes that contain polypeptide chains that repeatedly interact, either to activate, inactivate, oxidize, reduce, stabilize, etc., with one another, that can be produced in association with oil bodies using the methods provided herein include those set forth in FIG. 5. Accordingly, exemplary proteins for use in the heteromultimeric-protein-complexes and nucleic acid constructs encoding such, provided herein include, among others described herein, those set forth in FIG. 5.

Other polypeptide regions that can be used in the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, or the first and/or second thioredoxin-related proteins, provided herein include, among other, those immunoglobulin regions set forth in Table 1.

TABLE 1
ANTIBODY HETERODIMERS

| Class or molecule | Subunits |
|---|---|
| Fab | Variable region and first constant region |
| | of heavy chain and complete light chain |
| Fv | Variable regions of heavy and light |
| | antibody chains |
| IgA | heavy chains, light chains and J (joining) |
| | chain |
| IgG, IgD, IgE | heavy and light chains |
| IgM | heavy chains, light chains and J (joining) |
| | chain |
| Antibody chain(s) and a toxin | Antibody chain(s) and a toxin |
| Autoantigens, allergens and | Autoantigens, allergens and transplant |
| transplant antigens with an | antigens with an adjuvant or tolerogen |
| adjuvant or tolerogen | |
| Chimeras using antibody Fc | Receptor subunits fused to the constant |
| domain | region of antibody heavy chains |

As set forth above, in one embodiment, exemplary heteromultimeric-protein-complexes and exemplary heteromultimeric-fusion-proteins provided herein comprise redox proteins, such as the thioredoxins and thioredoxin-reductases and immunoglobulins.

Oil-Body-Targeting-Proteins

The nucleic acid sequence encoding the oil-body-targeting-protein that may be used in the methods and compositions provided herein may be any nucleic acid sequence encoding an oil-body-targeting-protein, protein fragment or peptide capable of association with first recombinant polypeptide, heteromultimeric-protein-complexes, multimeric-fusion-proteins, heteromultimeric-fusion-proteins, immunoglobulins, immunoglobulin-polypeptide-chains, redox-fusion-polypeptides, and/or a first and/or second thioredoxin-related protein and the oil bodies. The nucleic acid sequence encoding the oil body targeting peptide may be synthesized or obtained from any biological source.

For example, in one embodiment the oil-body-targeting-protein is an immunoglobulin or an immunoglobulin derived molecule, for example, a bispecific single chain antibody. The generation of single chain antibodies and bi-specific single chain antibodies is known to the art (see, e.g., U.S. patents U.S. Pat. No. 5,763,733, U.S. Pat. No. 5,767,260 and U.S. Pat. No. 5,260,203). Nucleic acid sequences encoding single chain antibodies functioning as oil-body-targeting-proteins may be prepared from hybridoma cell lines expressing monoclonal antibodies raised against an oleosin as described by Alting-Mees et al (2000) IBC's Annual International Conference on Antibody Engineering, Poster #1. In order to attain specificity for the first recombinant polypeptide a nucleic acid sequence encoding a second single chain antibody prepared from a monoclonal raised against the first recombinant polypeptide may be prepared and linked to the anti-oleosin single chain antibody. In this embodiment the oil body associates with the first recombinant polypeptide through non-covalent interactions of the oil-body-targeting-protein with the first recombinant polypeptide and the oil body. Alternatively the first recombinant polypeptide may be prepared as a fusion protein with an oil-body-targeting-protein. For example, a nucleic acid sequence encoding a single chain antibody raised against an oleosin may be fused to a nucleic acid sequence encoding the first recombinant polypeptide

Non-immunoglobulin-based oil-body-targeting-proteins capable of association with the first recombinant polypeptide may be discovered and prepared using for example phage display techniques (Pharmacia Biotech Catalogue Number 27-9401-011 Recombinant Phage Antibody System Expression Kit).

Oil-body-targeting-proteins may also be chemically modified. For example, oleosins may be modified by changing chemical modification of the lysine residues using chemical agents such as biotinyl-N-hyrdoxysuccinimide ester resulting in a process referred to as biotinylation. Conveniently this is accomplished by in vitro biotinylation of the oil bodies. In vivo biotinylation may be accomplished using the biotinylation domain peptide from the biotin carboxy carrier protein of E. coli acetyl-CoA carboxylase (Smith et al. (1998) Nucl. Acids. Res. 26: 1414-1420). Avidin or streptavidin may subsequently be used to accomplish association of the redox protein with the oil body.

In a particular embodiment the oil-body-targeting-protein is an oil-body-protein such as for example an oleosin or a caleosin or a sufficient portion derived thereof capable of targeting to an oil body. Nucleic acid sequences encoding oleosins are known to the art. These include for example the Arabidopsis oleosin (van Rooijen et al (1991) Plant Mol. Bio. 18:1177-1179); the maize oleosin (Qu and Huang (1990) J. Biol. Chem. Vol. 265 4:2238-2243); rapeseed oleosin (Lee and Huang (1991) Plant Physiol. 96:1395-1397); and the carrot oleosin (Hatzopoulos et al (1990) Plant Cell Vol. 2, 457-467.). Caleosin nucleic acid sequences are also known to the art (Naested et al (2000) Plant Mol Biol. 44(4):463-476; Chen et al (1999) Plant Cell Physiol. 40(10):1079-1086). Animal cell derived oil body proteins that may be used herein include adopihilin (Brasaemle et al, (1997) J. Lipid Res., 38: 2249-2263; Heid et al. (1998) Cell Tissue Research 294: 309-321), perilipin (Blanchette-Mackie et al. (1995), J. Lipid Res. 36: 1211-1226; Servetnick et al. (1995) J. Biol. Chem. 270: 16970-16973), apolipoproteins such as apo A-I, A-II, A-IV, C-I, C-II, CIII (Segrest et al. (1990), Proteins 8:103-117) and apoB (Chatterton et al. (1995) J. Lipid Res. 36: 2027-2037; Davis, R A in: Vance D E, Vance J. editors. Lipoprotein structure and secretion. The Netherlands, Elsevier, 191: 403-426.

In one embodiment, the first recombinant polypeptide is fused to an oil-body-protein. The methodology is further described in U.S. Pat. No. 5,650,554, which is incorporated herein by reference in its entirety. The first recombinant polypeptide may be fused to the N-terminus as well as to the C-terminus of the oil-body-protein (as described in: Moloney and van Rooijen (1996) INFORM 7:107-113) and fragments of the oil-body-protein such as for example the central domain of an oleosin molecule, or modified versions of the oil-body-protein may be used. In this embodiment, the second recombinant polypeptide is expressed intracellularly and then intracellularlly associates with the first recombinant polypeptide to form the multimeric-protein-complex in the cell. Oil bodies comprising the multimeric-protein-complex are then conveniently isolated from the cells.

In a further embodiment both the first and second recombinant polypeptide are separately fused to an oil-body-protein. In this embodiment nucleic acid sequences encoding the first and second polypeptides may be prepared separately and introduced in separate cell lines or they may be introduced in the same cell lines. Where the nucleic acid sequences are introduced in the same cell line, these nucleic acid sequence may be prepared using two separate expression vectors, or they may be prepared using a single vector comprising nucleic acid sequences encoding both the first polypeptide fused to an body protein and the second polypeptide fused to an oil-body-protein. Where separate cell lines are used subsequent mating of the offspring (e.g. mating of plants) is used to prepare a generation of cells comprising oil bodies which comprise both the first and second recombinant polypeptide fused to an oil-body-protein.

In further alternate embodiment, the first and second recombinant polypeptide are fused to form a multimeric-fusion-protein comprising the multimeric-protein-complex. In such an embodiment, the first and second polypeptide is associated with the oil body through an oil-body-targeting-protein capable of associating with both the fusion protein and with the oil body. In a particular embodiment, the fusion protein comprising the multimeric-protein-complex is fused to an oil-body-protein, for example, an oleosin or caleosin.

In embodiments provided herein in which the multimeric-protein-complex is an immunoglobulin (e.g., a multimeric-immunoglobulin-complex), a particularly preferred oil body targeting protein is an oleosin or caleosin associated with an immunoglobulin binding protein, such as for example protein A (U.S. Pat. No. 5,151,350), protein L (U.S. Pat. No. 5,965,390) and protein G (U.S. Pat. No. 4,954,618), or active fragments of such immunoglobulin binding proteins.

New oil-body-proteins may be discovered for example by preparing oil bodies (described in further detail below) and identifying proteins in these preparations using for example SDS gel electrophoresis. Polyclonal antibodies may be raised against these proteins and used to screen cDNA libraries in order to identify nucleic acid sequences encoding oil-body-proteins. The methodologies are familiar to the skilled artisan (Huynh et al. (1985) in DNA Cloning Vol. 1. a Practical Approach ed. D M Glover, IRL Press, pp 49-78). New oil-body-proteins may further be discovered using known nucleic acid sequences encoding oil-body-proteins (e.g. the Arabidopsis, rapeseed, carrot and corn nucleic acid sequences) to probe for example cDNA and genomic libraries for the presence of nucleic acid sequences encoding oil-body-proteins.

In one embodiment, the first and second polypeptide are a first and second redox protein. Accordingly, one embodiment provided herein relates to novel and improved methods for the production of redox proteins. It has unexpectedly been found that a redox protein when prepared as a fusion protein with a second redox protein is fully enzymatically active when produced in association with an oil body. In contrast, when the redox protein is prepared without the second redox protein it has reduced enzymatic activity. In one embodiment, the first redox protein is at least 5 times more active when produced as a redox fusion polypeptide relative to production as a non-fusion polypeptide.

Accordingly, provided herein are methods for producing an oil body associated with a heteromultimeric redox protein complex, said method comprising:

(a) producing in a cell comprising oil bodies, a first redox protein and a second redox protein wherein said first redox protein is capable of interacting with said second redox protein, preferably in the cell, to form said heteromultmeric redox protein complex; and

(b) associating said heteromultimeric redox protein complex with an oil body through an oil-body-targeting-protein capable of associating with said oil bodies and said heteromultimeric redox protein complex.

In a particular embodiment the first and second redox protein are prepared as a fusion protein to form a redox fusion polypeptide. Accordingly, provided herein are methods for preparing an enzymatically active redox protein associated with oil bodies comprising:

a) producing in a cell a redox fusion polypeptide comprising a first redox protein linked to a second redox protein;

b) associating said redox fusion polypeptide with oil bodies through an oil-body-targeting-protein capable of associating with said redox fusion polypeptide and said oil bodies; and

c) isolating said oil bodies associated with said redox fusion polypeptide. The oil bodies in association with the redox protein may be used to prepare a variety of useful emulsions.

As used herein the phrase “redox proteins” or grammatical variations thereof, refers to any protein or active protein fragment capable of participating in electron transport. For example, redox proteins are capable of catalyzing the transfer of an electron donor (also frequently referred to as the reducing agent) to an electron acceptor (also frequently referred to as the oxidizing agent). In the process of electron transfer, the reducing agent (electron donor) is oxidized and the oxidizing agent (electron acceptor) is reduced. Exemplary redox proteins for use herein include iron-sulfur proteins, cytochromes, redox active thiol proteins and redox-active flavoproteins. To carry out their function as conduits for electron donors, redox proteins, such as thioredoxin and thioredoxin-reductase for example, are known to function by interacting or associating with one another in multimeric-protein-complexes (e.g., heteromultimeric-protein-complexes).

The term “redox fusion polypeptide” as used herein refers to any fusion polypeptide comprising a first redox protein linked to a second redox protein (e.g., an in-frame translational fusion). The redox proteins that may be used with the methods and compositions provided herein may be any redox protein. In one embodiment the first and second redox proteins are a pair of redox proteins that would normally occur together from the same source, in nature. In a particular embodiment, the first redox protein is a thioredoxin and the second redox protein is a thioredoxin-reductase.

The redox fusion polypeptide may be produced in any cell comprising oil bodies, including any animal cell, plant cell, algae cell, fungal cell or bacterial cell. In certain embodiments the redox fusion polypeptide is produced in a plant cell and in particular embodiments the redox fusion polypeptide is produced in the seed cells of a seed plant.

In particular embodiments the oil-body-targeting-protein that is used is an oil-body-protein. In embodiments of the present invention in which an oil-body-protein is used, the first and second redox protein are preferably covalently fused to the oil-body-protein. Accordingly, provided herein are methods for the preparation of a redox protein in association with an oil body comprising:

a) introducing into a cell a chimeric nucleic acid sequence comprising:

1) a first nucleic acid sequence capable of regulating transcription in said cell operatively linked to;

2) a second nucleic acid sequence encoding a recombinant fusion polypeptide comprising (i) a first nucleic acid sequence encoding a sufficient portion of an oil-body-protein to provide targeting of said recombinant fusion polypeptide to an oil body linked in reading frame to (ii) a second nucleic acid sequence encoding a redox fusion polypeptide comprising a first redox protein linked to a second redox protein operatively linked to;

3) a third nucleic acid sequence capable of terminating transcription in said cell;

b) growing said cell under conditions to permit expression of said redox fusion polypeptide in a progeny cell comprising oil bodies; and

c) isolating said oil bodies comprising said redox fusion polypeptide from said progeny cell.

Redox Proteins

In accordance with various methods and compositions provided herein, any nucleic acid sequence encoding a redox protein may be used. The nucleic acid sequence encoding the first and/or second redox protein may be obtained from any biological source or may be prepared synthetically. In general, nucleic acid sequences encoding redox proteins are well known in the art and readily available. See, for example: Cristiano et al. (1993) Genomics 17: (2) 348-354, Doyama et al. (1998) Plant Sci. 137: 53-62, Hoeoeg et al. (1984) Biosci. Rep. 4: 917-923; as well as the Swiss Protein sequences set forth in Table 5. Known nucleic acid sequences encoding redox proteins may be used to design and construct nucleic acid sequence based probes in order to uncover and identify previously undiscovered nucleic acid sequences encoding redox proteins, for example by screening cDNA or genomic libraries. Thus, additional nucleic acid sequences may be discovered and used in accordance with the present invention.

The nucleic acid sequence encoding the first and/or second redox protein may be obtained from separate sources or may be obtained from the same source. In general however, the nucleic acid sequence encoding a redox-fusion polypeptide comprises nucleic acid sequences encoding a first and a second redox protein obtained from the same or a similar biological source. In certain embodiments provided herein, wherein the nucleic acid sequence encoding the first and second redox protein is obtained from the same source, the nucleic acid sequence encoding the first redox protein and second redox protein may be naturally fused. In accordance with a particular embodiment, the nucleic acid sequences encoding the first and second redox protein are preferably obtained from a plant source.

As set forth above, a polypeptide spacer or linker of variable length may separate the first and second redox proteins from each other and/or from the oil-body-targeting-protein; and additional redox proteins (e.g., one or more) may be fused to the first and/or second redox protein.

The nucleic acid sequences encoding the redox proteins may be altered to improve expression levels for example by optimizing the nucleic acids sequence in accordance with the preferred codon usage for the particular cell type which is selected for expression of the redox proteins, or by altering of motifs known to destabilize mRNAs (see for example: PCT Patent Application 97/02352). Comparison of the codon usage of the redox protein with codon usage of the host will enable the identification of codons that may be changed. For example, typically plant evolution has tended towards a preference for CG rich nucleotide sequences while bacterial evolution has resulted in bias towards AT rich nucleotide sequences. By modifying the nucleic acid sequences to incorporate nucleic acid sequences preferred by the host cell, expression may be optimized. Construction of synthetic genes by altering codon usage is described in for example PCT patent Application 93/07278. The redox proteins may be altered using for example, targeted mutagenesis, random mutagenesis (Shiraishi et al. (1998) Arch. Biochem. Biophys. 358: 104-115; Galkin et al. (1997) Protein Eng. 10: 687-690; Carugo et al. (1997) Proteins 28: 10-28; Hurley et al. (1996) Biochemistry 35: 5670-5678) (and/or by the addition of organic solvent (Holmberg et al. (1999) Protein Eng. 12: 851-856). The polypeptide spacer between the first and second redox protein may be altered in similar ways.

The first and second redox protein may be selected by developing a two-dimensional matrix and determining which combination of first and second redox protein is most effective in electron transport using for example, a colorometric reduction assay (Johnson et al (1984) J. of Bact. Vol. 158 3:1061-1069, Luthman et al (1982) Biochemistry Vol 21 26:6628-2233). Combinations of thioredoxin and thioredoxin-reductase may be tested by determining the reduction of wheat storage proteins and milk storage protein beta-lactoglobulin in vitro (Del Val et al. (1999) J. Allerg. Clin. Immunol. 103: 690-697). Using the same strategy polypeptide spacers between the first and second redox proteins may be evaluated for their efficiency.

First and second redox proteins that may be used herein include without limitation any first redox protein and second redox protein selected from the group of redox proteins consisting of cytochromes, such as cytochrome a, cytochrome b and cytochrome c; porphyrin containing proteins, for example haemoglobin; iron-sulfur proteins, such as ferredoxin; flavoproteins such as thioredoxin-reductase, NADH dehydrogenase, succinate dehydrogenase, dihydrolipoyl dehydrogenase, acyl-CoA dehydrogenase, D-amino acid oxidase, xanthine oxidase, orotate reductase and aldehyde oxidase; pyridine-linked dehydrogenases, for example, lactate dehydrogenase, glyceraldehyde-3-phosphate dehydrogenase, malate dehydrogenase, and beta-hydroxy-butarate dehydrogenase; and redox active thiol containing proteins such as thioredoxin.

In particular embodiments, the redox proteins provided herein are thioredoxin and its reductant thioredoxin-reductase (which are jointly also referred to herein as “thioredoxin-related” protein(s)). As used herein, the term “thioredoxin” refers to relatively small proteins (typically approximately 12 kDa) that belong to the family of thioltransferases which catalyze oxido-reductions via the formation or hydrolysis of disulfide bonds and are widely, if not universally, distributed throughout the animal plant and bacterial kingdom. The reduces form of thioredoxin is an excellent catalyst for the reduction of even the most intractable disulfide bonds. In order to reduce the oxidized thioredoxin, two cellular reductants provide the reduction equivalents: reduced ferredoxin and NADPH. These reduction equivalents are supplied to thioredoxin via interaction or association with different thioredoxin-reductases including the NADPH thioredoxin-reductase and ferredoxin thioredoxin-reductase. The supply of these reduction equivalents requires the formation of a heteromultimeric-protein-complex comprising thioredoxin and thioredoxin-reductase. Ferredoxin thioredoxin-reductase is involved in the reduction of plant thioredoxins designated as Trxf and Trxm, both of which are involved in the regulation of photosynthetic processes in the chloroplast. The NADPH/thioredoxin active in plant seeds is designated Trxh (also referred to herein as thioredoxin h-type) and is capable of the reduction of a wide range of proteins thereby functioning as an important cellular redox buffer. Generally, only one kind of thioredoxin, which analogous to the plant Trxh type, is found in bacterial or animal cells. The h-type thioredoxins are capable of being reduced by NADPH and NADPH-thioredoxin reductase.

Exemplary thioredoxins are further characterized as a protein having a core of 5 beta-sheets surrounded by 4 to 6 alpha helixes. Exemplary thioredoxins are further characterized by having an active site containing the consensus amino acid sequence:

X C Y Y C Z,

wherein Y is any amino acid, such as hydrophobic or non-polar amino acids,

wherein X can be any of the 20 amino acids, preferably a hydrophobic amino acid, such as a tryptophan, and

Z can be any amino acid, preferably polar amino acids.

In certain embodiments, the thioredoxins for use herein comprise an active site having the amino acid sequence X C G P C Z. When the cysteines in the active site of thioredoxin or thioredoxin-like proteins are oxidized, they form an intramolecular disulfide bond. In the reduced state, the same active sites are capable of participating in redox reactions through the reversible oxidation of its active site dithiol, to a disulfide and catalyzes dithioldisulfide exchange reactions.

Exemplary thioredoxins are well-known in the art and can be obtained from several organisms including Arabidopsis thaliana (Riveira Madrid et al. (1995) Proc. Natl. Acad. Sci. 92: 5620-5624), wheat (Gautier et al. (1998) Eur. J. Biochem. 252: 314-324); Escherichia coli (Hoeoeg et al (1984) Biosci. Rep. 4: 917-923) and thermophylic microorganisms such as Methanococcus jannaschii and Archaeoglobus fulgidus (PCT Patent Application 00/36126). Thioredoxins have also been recombinantly expressed in several host systems including bacteria (Gautier et al. (1998) Eur J. Biochem. 252: 314-324) and plants (PCT Patent Application WO 00/58453) Commercial preparations of E. coli sourced Thioredoxins are readily available from for example: Sigma Cat No. T 0910 Thioredoxin (E. coli, recombinant; expressed in E. coli).

Exemplary nucleic acid sequences encoding thioredoxin polypeptides for use herein are readily available from a variety of diverse biological sources including E. coli (Hoeoeg et al. (1984) Biosci. Rep.: 4 917-923); Methanococcus jannaschii and Archaeoglobus fulgidus (PCT Patent Application 00/361 26); Arabidopsis thaliana (Rivera-Madrid (1995) Proc. Natl. Acad. Sci. 92: 5620-5624); wheat (Gautier et al (1998) Eur. J. Biochem. 252(2): 314-324); tobacco (Marty et al. (1991) Plant Mol. Biol. 17: 143-148); barley (PCT Patent Application 00/58352); rice (Ishiwatari et al. (1995) Planta 195: 456-463); soybean (Shi et al. (1996) Plant Mol. Biol. 32: 653-662); rapeseed (Bower et al. Plant Cell 8: 1641-1650) and calf (Terashima et al. (1999) DNA Seq. 10(3): 203-205); and the like. In yet other embodiments, exemplary nucleic acids for use herein include those encoding the thioredoxin and thioredoxin-like polypeptide chains set forth as SEQ ID NOs:38, 42, 46 and 50; and those encoding the thioredoxin and thioredoxin-like polypeptide chains set forth in Table 5 as SEQ ID NOs:52-194. The respective nucleic acid sequences encoding the amino acids set forth in SEQ ID NOs:52-194 can be readily identified via the Swiss Protein identifier (accession) numbers provided in Table 5 (in parenthesis).

As used herein, the term “thioredoxin-reductase” refers to a protein that complexes with a flavin, such as FAD. The flavin compound serves as an electron donor for the thioredoxin-reductase protein active site. Thioredoxin reductases have a redox active, disulfide bond site capable of reducing thioredoxin. The active site of thioredoxin-reductase contains 2 cysteines. The type of amino acids surrounding the 2 cysteine residues forming the active site can vary as hydrophobic, non-polar or polar. An exemplary thioredoxin-reductase is NADPH-thioredoxin-reductase (TR), which is a cytosolic homodimeric enzyme comprising typically 300-500 amino acids. Crystal structures of both E. coli and plant thioredoxin-reductase have been obtained (Waksman et al. (1994) J. Mol. Biol. 236: 800-816; Dai et al. (1996) J. Mol. Biol. 264:1044-1057). NADPH-thioredoxin-reductases have been expressed in heterologous hosts, for example the Arabidopsis NADPH-thioredoxin-reductase has been expressed in E. coli (Jacquot et al. (1 994) J. Mol. Biol. 235: 1357-1363) and wheat (PCT Patent Application 00/58453).

Exemplary nucleic acid sequences encoding thioredoxin-reductase proteins can readily be obtained from a variety of sources, such as from the sequence set forth in Table 5 and the Sequence Listing provide herein, from Arabidopsis (Riveira Madrid et al. (1995) Proc. Natl. Acad. Sci. USA 92: 5620-5624), E. coli (Russel et al. (1988) J. Biol. Chem. 263: 9015-9019); barley (PCT Patent Application 00/58352 and wheat (Gautier et al., (1998) Eur. J. Biochem. 252: 314-324); and the like. In yet other embodiments, exemplary nucleic acids for use herein include those encoding the thioredoxin-reductase polypeptide chains set forth as SEQ ID NOs:8, 9, 10, 40, 44, 48 and 50; and those encoding the thioredoxin-reductase polypeptide chains set forth in Table 5 as SEQ ID NOs:195-313. The respective nucleic acid sequences encoding the amino acids set forth in SEQ ID NOs:195-313 can be readily identified via the Swiss Protein identifier (accession) numbers provided in Table 5 (in parenthesis).

Also contemplated for use in the methods and compositions provided herein are nucleic acid and amino acid homologs that are “subtantially homologous” to the thioredoxin and thioredoxin-reductase nucleic and amino acids set forth herein, which includes thioredoxin and thioredoxin-reductase polypeptides encoded by a sequence of nucleotides that hybridizes under conditions of low, moderate or high stringency to the sequence of nucleotides encoding the thioredoxin and thioredoxin-reductase nucleic and amino acids set forth herein (e.g., in the Examples, Sequence Listing and/or Table 5). As used herein, a DNA or nucleic acid homolog refers to a nucleic acid that includes a preselected conserved nucleotide sequence, such as a sequence encoding a therapeutic polypeptide. By the term “substantially homologous” is meant having at least 80%, preferably at least 90%, most preferably at least 95% homology therewith or a less percentage of homology or identity and conserved biological activity or function.

The terms “homology” and “identity” are often used interchangeably. In this regard, percent homology or identity may be determined, for example, by comparing sequence information using a GAP computer program. The GAP program utilizes the alignment method of Needleman and Wunsch (J. Mol. Biol. 48:443 (1970), as revised by Smith and Waterman (Adv. Appl. Math. 2:482 (1981). Briefly, the GAP program defines similarity as the number of aligned symbols (i.e., nucleotides or amino acids) which are similar, divided by the total number of symbols in the shorter of the two sequences. The preferred default parameters for the GAP program may include: (1) a unary comparison matrix (containing a value of 1 for identities and 0 for non-identities) and the weighted comparison matrix of Gribskov and Burgess, Nucl. Acids Res. 14:6745 (1986), as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

By sequence identity, the number of conserved amino acids are determined by standard alignment algorithms programs, and are used with default gap penalties established by each supplier. Substantially homologous nucleic acid molecules would hybridize typically at moderate stringency or at high stringency all along the length of the nucleic acid of interest. Preferably the two molecules will hybridize under conditions of high stringency. Also contemplated are nucleic acid molecules that contain degenerate codons in place of codons in the hybridizing nucleic acid molecule.

Whether any two nucleic acid molecules have nucleotide sequences that are at least 80%, 85%, 90%, 95%, 96%, 97%, 98% or 99% “identical” can be determined using known computer algorithms such as the “FAST A” program, using for example, the default parameters as in Pearson and Lipman, Proc. Natl. Acad. Sci. USA 85:2444 (1988). Alternatively the BLAST function of the National Center for Biotechnology Information database may be used to determine relative sequence identity.

In general, sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Preferred computer program methods to determine identity and similarity between two sequences include, but are not limited to, GCG program package (Devereux, J., et al., Nucleic Acids Research 12(I):387 (1984)), BLASTP, BLASTN, FASTA (Atschul, S. F., et al., J Molec Biol 215:403 (1990)).

Therefore, as used herein, the term “identity” represents a comparison between a test and a reference polypeptide or polynucleotide. For example, a test polypeptide may be defined as any polypeptide that is 90% or more identical to a reference polypeptide.

As used herein, the term at least “90% identical to” refers to percent identities from 90 to 99.99 relative to the reference polypeptides. Identity at a level of 90% or more is indicative of the fact that, assuming for exemplification purposes a test and reference polynucleotide length of 100 amino acids are compared. No more than 10% (i.e., 10 out of 100) amino acids in the test polypeptide differs from that of the reference polypeptides. Similar comparisons may be made between a test and reference polynucleotides. Such differences may be represented as point mutations randomly distributed over the entire length of an amino acid sequence or they may be clustered in one or more locations of varying length up to the maximum allowable, e.g. 10/100 amino acid difference (approximately 90% identity). Differences are defined as nucleic acid or amino acid substitutions, or deletions.

As used herein: stringency of hybridization in determining percentage mismatch is as follows:

1) high stringency: 0.1×SSPE, 0.1% SDS, 65° C.

2) medium stringency: 0.2×SSPE, 0.1% SDS, 50° C.

3) low stringency: 1.0×SSPE, 0.1% SDS, 50° C.

Those of skill in this art know that the washing step selects for stable hybrids and also know the ingredients of SSPE (see, e.g., Sambrook, E. F. Fritsch, T. Maniatis, in: Molecular Cloning, A Laboratory Manual, Cold Spring Harbor Laboratory Press (1989), vol. 3, p. B.13, see, also, numerous catalogs that describe commonly used laboratory solutions). SSPE is pH 7.4 phophate-buffered 0.18 NaCl. Further, those of skill in the art recognize that the stability of hybrids is determined by Tₘ, which is a function of the sodium ion concentration and temperature (Tₘ=81.5° C.−16.6(log₁₀[Na⁺])+0.41(%G+C)−600/I)), so that the only parameters in the wash conditions critical to hybrid stability are sodium ion concentration in the SSPE (or SSC) and temperature.

It is understood that equivalent stringencies may be achieved using alternative buffers, salts and temperatures. By way of example and not limitation, procedures using conditions of low stringency are as follows (see also Shilo and Weinberg, Proc. Natl. Acad. Sci. USA, 78:6789-6792 (1981)): Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA (10×SSC is 1.5 M sodium chloride, and 0.15 M sodium citrate, adjusted to a pH of 7).

In a particular embodiment, a heteromultimeric-protein-complex is produced as a fusion polypeptide between the first and second redox protein, wherein the first redox protein is thioredoxin and the second redox protein is a thioredoxin-reductase. In one embodiment, the second recombinant polypeptide, e.g., the thioredoxin-reductase is positioned N-terminal relative to the first recombinant polypeptide, e.g., the thioredoxin. Accordingly, any protein which is classified as thioredoxin, such as the thioredoxin component of the NADPH thioredoxin system and the thioredoxin present in the ferredoxin/thioredoxin system also known as TRx and TRm may be used in combination with any thioredoxin-reductase such as the NADPH thioredoxin-reductase and the ferredoxin-thioredoxin-reductase and any other proteins having the capability of reducing thioredoxin. In particular embodiments the thioredoxin and thioredoxin-reductase are plant derived.

In an alternate embodiment, the naturally occurring nucleic acid sequence encoding the thioredoxin/thioredoxin-reductase protein fusion obtainable from Mycobacterium leprae (Wieles et al. (1995) J. Biol. Chem. 27:25604-25606) is used, as set forth in the Examples herein.

Immunoglobulins

In another embodiment of the present invention, the multimeric-protein-complexes are immunoglobulins. As used herein “immunoglobulin-polypeptide-chain” refers to a first polypeptide that is capable of associating with a second polypeptide to form an immunologically active (i.e. capable of antigen binding) multimeric-protein-complex. The types of immunoglobulins and immunoglobulin-polypeptide-chains contemplated for use herein include the immunologically active (i.e. antigen binding) portions of a light and heavy chain. Exemplary immunoglobulins and immunoglobulin-polypeptide-chains for use herein include substantially intact immunoglobulins, including any IgG, IgA, IgD, IgE and IgM, as well as any portion of an immunoglobulin, including those portions well-known as Fab fragments, Fab′ fragments, F(ab′).sub2. fragments and Fv fragments.

In this embodiment, the first recombinant polypeptide may be any immunoglobulin heavy chain, including any IgG, IgA, IgD, IgE or IgM heavy chain, and the second recombinant polypeptide may be a kappa or lambda immunoglobulin light chain. Accordingly, provided herein are methods of producing an immunoglobulin, said method comprising: (a) producing in a cell comprising oil bodies, a first immunoglobulin-polypeptide-chain and a second immunoglobulin-polypeptide-chain wherein said first immunoglobulin-polypeptide-chain is capable of associating with said second immunoglobulin-polypeptide-chain to form said immunoglobulin; and (b) associating said immunoglobulin with an oil body through an oil-body-targeting-protein capable of associating with said oil bodies and said first immunoglobulin-polypeptide-chain.

As set forth herein, the multimeric immunoglobulin is associated with an oil body through an oil-body-targeting-protein. In particular embodiments, the oil-body-targeting-protein may be a fusion polypeptide comprising an oil-body-protein and an immunoglobulin binding protein, such as for example protein A, protein L, and protein G.

In yet another embodiment involving immunoglobulins, the first and second recombinant polypeptides (immunoglobulins) are separately fused to an oil body protein, for example an oleosin or caleosin. For example,

a) the first recombinant polypeptide may be an immunoglobulin heavy chain, including any IgG, IgA, IgD, IgE or IgM heavy chain, and the second recombinant polypeptide may be a kappa or lambda immunoglobulin light chain; or

b) the first recombinant polypeptide may be the variable and first constant domain from an immunoglobulin heavy chain and the second recombinant polypeptide may be a kappa or lambda immunoglobulin light chain; or

c) the first recombinant polypeptide may be the variable domain from an immunoglobulin heavy chain and the second recombinant polypeptide may be the variable domain from a kappa or lambda immunoglobulin light chain.

In certain embodiments, the fusion polypeptides are designed or selected to allow the heteromultimeric-protein-complex formation between immunoglobulin light and heavy chain sequences on the oil bodies within the cell comprising oil bodies.

Preparation of Expression Vectors Comprising Oil-Body-Targeting-Proteins and the First and/or Second Recombinant Polypeptides, Multimeric-Protein-Complexes, Heteromultimeric-Protein-Complexes, Multimeric-Fusion-Proteins, Heteromultimeric-Fusion-Proteins, Immunoglobulins, Immunoglobulin-Polypeptide-Chains, Redox-Fusion-Polypeptides, or the First and/or Second Thioredoxin-Related Proteins.

In accordance with the present invention, the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, or the first and/or second thioredoxin-related proteins; and the oil-body-targeting-protein are conveniently produced in a cell. In order to produce the recombinant polypeptides or multimeric-protein-complexes, a nucleic acid sequence encoding either the the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, or the first and/or second thioredoxin-related proteins; and/or the oil-body-targeting-protein are incorporated in a recombinant expression vector. Accordingly, provided herein are recombinant expression vectors comprising the chimeric nucleic acids provided herein suitable for expression of the oil-body-targeting-protein and the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, or the first and/or second thioredoxin-related proteins, suitable for the selected cell. The term “suitable for expression in the selected cell” means that the recombinant expression vector contains all nucleic acid sequences required to ensure expression in the selected cell.

Accordingly, the recombinant expression vectors further contain regulatory nucleic acid sequences selected on the basis of the cell which is used for expression and ensuring initiation and termination of transcription operatively linked to the nucleic acid sequence encoding the recombinant polypeptide or multimeric-protein-complex and/or the oil-body-targeting-protein. Regulatory nucleic acid sequences include promoters, enhancers, silencing elements, ribosome binding sites, Shine-Dalgarno sequences, introns and other expression elements. “Operatively linked” is intended to mean that the nucleic acid sequences comprising the regulatory regions linked to the nucleic acid sequences encoding the recombinant polypeptide or multimeric-protein-complex and/or the oil-body-targeting-protein allow expression in the cell. A typical nucleic acid construct comprises in the 5′ to 3′ direction a promoter region capable of directing expression, a coding region comprising the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, or the first and/or second thioredoxin-related proteins; and/or an oil-body-targeting-protein and a termination region functional in the selected cell.

The selection of regulatory sequences will depend on the organism and the cell type in which the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, or the first and/or second thioredoxin-related proteins; and/or the oil-body-targeting-protein is expressed, and may influence the expression levels of the polypeptide. Regulatory sequences are art-recognized and selected to direct expression of the oil-body-targeting-protein and the recombinant polypeptides or multimeric-protein-complexes in the cell.

Promoters that may be used in bacterial cells include the lac promoter (Blackman et al. (1978) Cell: 13: 65-71), the trp promoter (Masuda et al. (1996) Protein Eng: 9: 101-106) and the T7 promoters (Studier et al. (1986) J. Mol. Biol. 189: 113-130). Promoters functional in plant cells that may be used herein include constitutive promoters such as the 35S CaMV promoter (Rothstein et al. (1987) Gene: 53: 153-161) the actin promoter (McElroy et al. (1990) Plant Cell 2: 163-171) and the ubiquitin promoter (European Patent Application 0 342 926). Other promoters are specific to certain tissues or organs (for example, roots, leaves, flowers or seeds) or cell types (for example, leaf epidermal cells, mesophyll cells or root cortex cells) and or to certain stages of plant development. Timing of expression may be controlled by selecting an inducible promoter, for example the PR-a promoter described in U.S. Pat. No. 5,614,395. Selection of the promoter therefore depends on the desired location and timing of the accumulation of the desired polypeptide. In a particular embodiment, the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, or the first and/or second thioredoxin-related proteins; and the oil-body-targeting-protein are expressed in a seed cell and seed specific promoters are utilized. Seed specific promoters that may be used herein include for example the phaseolin promoter (Sengupta-Gopalan et al. (1985) Proc. Natl. Acad. Sci. USA: 82: 3320-3324), and the Arabidopsis 18 kDa oleosin promoter (van Rooijen et al. (1992) Plant. Mol. Biol. 18: 1177-1179). New promoters useful in various plant cell types are constantly discovered. Numerous examples of plant promoters may be found in Ohamuro et al. (Biochem of Pl. (1989) 15: 1-82).

Genetic elements capable of enhancing expression of the polypeptide may be included in the expression vectors. In plant cells these include for example, the untranslated leader sequences from viruses such as the AMV leader sequence (Jobling and Gehrke (1987) Nature: 325: 622-625) and the intron associated with the maize ubiquitin promoter (See: U.S. Pat. No. 5,504,200).

Transcriptional terminators are generally art recognized and besides serving as a signal for transcription termination serve as a protective element serving to extend the mRNA half-life (Guarneros et al. (1982) Proc. Natl. Acad. Sci. USA: 79: 238-242). In nucleic acid sequences for the expression in plant cells, the transcriptional terminator typically is from about 200 nucleotide to about 1000 nucleotides in length. Terminator sequences that may be used herein include for example, the nopaline synthase termination region (Bevan et al. (1983) Nucl. Acid. Res.: 11: 369-385), the phaseolin terminator (van der Geest et al. (1994) Plant J.: 6: 413-423), the terminator for the octopine synthase gene of Agrobacterium tumefaciens or other similarly functioning elements. Transcriptional terminators can be obtained as described by An (1987) Methods in Enzym. 153: 292). The selection of the transcriptional terminator may have an effect on the rate of transcription.

Accordingly, provided herein are chimeric nucleic acid sequences encoding a first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, and/or thioredoxin-related proteins. In one embodiment, said nucleic acid comprises:

(a) a first nucleic acid sequence encoding an oil-body-targeting-protein operatively linked in reading frame to;

(b) a second nucleic acid sequence encoding a first recombinant polypeptide, immunoglobulin-polypeptide-chain, or redox protein; linked in reading frame to;

(c) a third nucleic acid sequence encoding a second recombinant polypeptide, immunoglobulin-polypeptide-chain or redox protein, wherein said first and second recombinant polypeptides, immunoglobulin-polypeptide-chains or redox proteins are capable of forming a multimeric-protein-complex.

In another embodiment, provided herein is an expression vector comprising:

1) a first nucleic acid sequence capable of regulating transcription in said cell operatively linked to;

2) a second nucleic acid sequence encoding a recombinant fusion polypeptide comprising (i) a nucleic acid sequence encoding a sufficient portion of an oil-body-protein to provide targeting of said recombinant fusion polypeptide to an oil body linked in reading frame to (ii) a nucleic acid sequence encoding a multimeric-fusion-protein, such as a redox fusion polypeptide or immunoglobulin, comprising a first recombinant polypeptide, such as a redox protein or immunoglobulin-polypeptide-chain, linked to a second recombinant polypeptide, such as a second redox protein or a second immunoglobulin-polypeptide-chain, operatively linked to;

3) a third nucleic acid sequence capable of terminating transcription in said cell.

The recombinant expression vector further may contain a marker gene. Marker genes that may be used in accordance with the present invention include all genes that allow the distinction of transformed cells from non-transformed cells including all selectable and screenable marker genes. A marker may be a resistance marker such as an antibiotic resistance marker against for example kanamycin, ampicillin, G418, bleomycin hygromycin, chloramphenicol which allows selection of a trait by chemical means or a tolerance marker against for example a chemical agent such as the normally phytotoxic sugar mannose (Negrotto et al. (2000) Plant Cell Rep. 19: 798-803). In plant recombinant expression vectors herbicide resistance markers may conveniently be used for example markers conferring resistance against glyphosate (U.S. Pat. Nos. 4,940,935 and 5,188,642) or phosphinothricin (White et al. (1990) Nucl. Acids Res. 18: 1062; Spencer et al. (1990) Theor. Appl. Genet. 79: 625-631). Resistance markers to a herbicide when linked in close proximity to the redox protein or oil-body-targeting-protein may be used to maintain selection pressure on a population of plant cells or plants for those plants that have not lost the protein of interest. Screenable markers that may be employed to identify transformants through visual observation include beta-glucuronidase (GUS) (see U.S. patents U.S. Pat. No. 5,268,463 and U.S. Pat. No. 5,599,670) and green fluorescent protein (GFP) (Niedz et al. (1995) Plant Cell Rep.: 14: 403).

The recombinant expression vectors further may contain nucleic acid sequences encoding targeting signals ensuring targeting to a cell compartment or organelle. Suitable targeting signals that may be used herein include those that are capable of targeting polypeptides to the endomembrane system. Exemplary targeting signals that may be used herein include targeting signals capable of directing the protein to the periplasm, the cytoplasm, the golgi apparatus, the apoplast (Sijmons et al., 1990, Bio/Technology, 8:217-221) the chloroplast (Comai et al. (1988) J. Biol. Chem. 263: 15104-15109), the mitochondrion, the peroxisome (Unger et al. (1989) Plant Mol. Biol. 13: 411-418), the ER, the vacuole (Shinshi et al. (1990) Plant Mol. Biol. 14: 357-368 and the oil body. By the inclusion of the appropriate targeting sequences it is possible to direct the oil-body-targeting-protein or the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, and/or thioredoxin-related proteins, to the desired organelle or cell compartment.

The recombinant expression vectors of the present invention may be prepared in accordance with methodologies well known to those of skill in the art of molecular biology (see for example: Sambrook et al. (1990) Molecular Cloning, 2^{nd} ed. Cold Spring Harbor Press). The preparation of these constructs may involve techniques such as restriction digestion, ligation, gel electrophoresis, DNA sequencing and PCR. A wide variety of cloning vectors is available to perform the necessary cloning steps resulting in a recombinant expression vector ensuring expression of the polypeptide. Especially suitable for this purpose are vectors with a replication system that is functional in Escherichia coli such as pBR322, the PUC series of vectors, the M13mp series of vectors, pBluescript etc. Typically these vectors contain a marker allowing the selection of transformed cells for example by conferring antibiotic resistance. Nucleic acid sequences may be introduced in these vectors and the vectors may be introduced in E. coli grown in an appropriate medium. Vectors may be recovered from cells upon harvesting and lysing the cells.

Recombinant expression vectors suitable for the introduction of nucleic acid sequences in plant cells include Agrobacterium and Rhizobium based vectors such as the Ti and Ri plasmids. Agrobacterium based vectors typically carry at least one T-DNA border sequence and include vectors such pBIN 19 (Bevan (1984) Nucl Acids Res. Vol. 12, 22:8711-8721) and other binary vector systems (for example: U.S. Pat. No. 4,940,838).

Production of Cells Comprising a First and/or Second Recombinant Polypeptides, Multimeric-Protein-Complexes, Heteromultimeric-Protein-Complexes, Multimeric-Fusion-Proteins, Heteromultimeric-Fusion-Proteins, Immunoglobulins, Immunoglobulin-Polypeptide-Chains, Redox-Fusion-Polypeptides, and/or a First and/or Second Thioredoxin-Related Protein and Oil-Body-Targeting-Proteins

In accordance with the present invention, the recombinant expression vectors are introduced into the cell that is selected and the selected cells are grown to produce the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, a first and/or second thioredoxin-related protein; and the oil-body-targeting-protein either directly or in a progeny cell.

Methodologies to introduce recombinant expression vectors into a cell also referred to herein as “transformation” are well known to the art and vary depending on the cell type that is selected. General techniques to transfer the recombinant expression vectors into the cell include electroporation; chemically mediated techniques, for example CaCl2 mediated nucleic acid uptake; particle bombardment (biolistics); the use of naturally infective nucleic acid sequences for example virally derived nucleic acid sequences or when plant cells are used Agrobacterium or Rhizobium derived nucleic acid sequences; PEG mediated nucleic acid uptake, microinjection, and the use of silicone carbide whiskers (Kaeppler et al. (1990) Plant Cell Rep. 9:415-418) all of which may be used herein.

Introduction of the recombinant expression vector into the cell may result in integration of its whole or partial uptake into host cell genome including the chromosomal DNA or the plastid genome. Alternatively the recombinant expression vector may not be integrated into the genome and replicate independently of the host cell's genomic DNA. Genomic integration of the nucleic acid sequence is typically used as it will allow for stable inheritance of the introduced nucleic acid sequences by subsequent generations of cells and the creation of cell, plant or animal lines.

Particular embodiments involve the use of plant cells. Particular plant cells used herein include cells obtainable from Brazil nut (Betholletia excelsa); castor (Riccinus communis); coconut (Cocus nucifera); coriander (Coriandrum sativum); cotton (Gossypium spp.); groundnut (Arachis hypogaea); jojoba (Simmondsia chinensis); linseed/flax (Linum usitatissimum); maize (Zea mays); mustard (Brassica spp. and Sinapis alba); oil palm (Elaeis guineeis); olive (Olea europaea); rapeseed (Brassica spp.); safflower (Carthamus tinctorius); soybean (Glycine max); squash (Cucurbita maxima); barley (Hordeum vulgare); wheat (Traeticum aestivum) and sunflower (Helianthus annuus).

Transformation methodologies for dicotelydenous plant species are well known. Generally Agrobacterium mediated transformation is utilized because of its high efficiency as well as the general susceptibility by many, if not all dicotelydenous plant species. Agrobacterium transformation generally involves the transfer of a binary vector (e.g. pBIN 19) comprising the DNA of interest to an appropriate Agrobacterium strain (e.g. CIB542) by for example tri-parental mating with an E. coli strain carrying the recombinant binary vector and an E. coli strain carrying a helper plasmid capable of mobilization of the binary vector to the target Agrobacterium strain, or by DNA transformation of the Agrobacterium strain (Hofgen et al. Nucl. Acids. Res. (1988) 16: 9877. Other transformation methodologies that may be used to transform dicotelydenous plant species include biolistics (Sanford (1988) Trends in Biotechn. 6: 299-302); electroporation (Fromm et al. (1985) Proc. Natl. Acad. Sci. USA 82: 5824-5828); PEG mediated DNA uptake (Potrykus et al. (1985) Mol. Gen. Genetics 199: 169-177); microinjection (Reich et al. Bio/Techn. (1986) 4: 1001-1004) and silicone carbide whiskers (Kaeppler et al. (1990) Plant Cell Rep. 9: 415-418). The exact transformation methodologies typically vary somewhat depending on the plant species that is used.

In a particular embodiment the oil bodies are obtained from safflower and the recombinant proteins are expressed in safflower. Safflower transformation has been described by Baker and Dyer (Plant Cell Rep. (1996) 16: 106-110).

Monocotelydenous plant species may now also be transformed using a variety of methodologies including particle bombardment (Christou et al. (1991) Biotechn. 9: 957-962; Weeks et al. Plant Physiol. (1993) 102: 1077-1084; Gordon-Kamm et al. Plant Cell (1990) 2: 603-618) PEG mediated DNA uptake (EP 0 292 435; 0 392 225) or Agrobacterium-mediated transformation (Goto-Fumiyuki et al (1999) Nature-Biotech. 17 (3):282-286).

Plastid transformation is decribed in U.S. Pat. Nos. 5,451,513; 5,545,817 and 5,545,818; and PCT Patent Applications 95/16783; 98/11235 and 00/39313) Basic chloroplast transformation involves the introduction of cloned plastid DNA flanking a selectable marker together with the nucleic acid sequence of interest into a suitable target tissue using for example biolistics or protoplast transformation. Selectable markers that may be used include for example the bacterial aadA gene (Svab et al. (1993) Proc. Natl. Acad. Sci. USA 90: 913-917). Plastid promoters that may be used include for example the tobacco clpP gene promoter (PCT Patent Application 97/06250).

In another embodiment, the invention chimeric nucleic acid contructs provided herein are directly transformed into the plastid genome. Plastid transformation technology is described extensively in U.S. Pat. Nos. 5,451,513, 5,545,817, 5,545,818 and 5,576,198; in PCT application nos. WO 95/16783 and WO 97/32977; and in McBride et. al., Proc Natl Acad Sci USA 91: 7301-7305 (1994), the entire disclosures of all of which are hereby incorporated by reference. In one embodiment, plastid transformation is achieved via biolistics; first carried out in the unicellular green alga Chlamydomonas reinhardtii (Boynton et al. (1988) Science 240:1534-1537)) and then extended to Nicotiana tabacum (Svab et al. (1990) Proc Natl Acad Sci USA 87:8526-8530), combined with selection for cis-acting antibiotic resistance loci (spectinomycin or streptomycin resistance) or complementation of non-photosynthetic mutant phenotypes.

In another embodiment, tobacco plastid transformation is carried out by particle bombardment of leaf or callus tissue, or polyethylene glycol (PEG)-mediated uptake of plasmid DNA by protoplasts, using cloned plastid DNA flanking a selectable antibiotic resistance marker. For example, 1 to 1.5 kb flanking regions, termed targeting sequences, facilitate homologous recombination with the plastid genome and allow the replacement or modification of specific regions of the 156 kb tobacco plastid genome. In one embodiment, point mutations in the plastid 16S rDNA and rps12 genes conferring resistance to spectinomycin and/or streptomycin can be utilized as selectable markers for transformation (Svab et al (1990) Proc Natl Acad Sci USA 87:8526-8530; Staub et al. (1992) Plant Cell 4:39-45, the entire disclosures of which are hereby incorporated by reference), resulting in stable homoplasmic transformants at a frequency of approximately one per 100 bombardments of target leaves. The presence of cloning sites between these markers allows creation of a plastid targeting vector for introduction of foreign genes (Staub et al. (1993) EMBO J 12:601-606, the entire disclosure of which is hereby incorporated by reference). In another embodiment, substantial increases in transformation frequency can be obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab et al. (1993) Proc Natl Acad Sci USA 90: 913-917, the entire disclosure of which is hereby incorporated by reference). This marker has also been used successfully for high-frequency transformation of the plastid genome of the green alga Chlamydomonas reinhardtii (Goldschmidt-Clermont, M. (1991) Nucl Acids Res 19, 4083-4089, the entire disclosure of which is hereby incorporated by reference). In other embodiments, plastid transformation of protoplasts from tobacco and the moss Physcomitrella can be attained using PEG-mediated DNA uptake (O'Neill et al (1993) Plant J 3:729-738; Koop et al. (1996) Planta 199:193-201, the entire disclosures of which are hereby incorporated by reference).

Both particle bombardment and protoplast transformation are also contemplated for use herein. Plastid transformation of oilseed plants has been successfully carried out in the genera Arabidopsis and Brassica (Sikdar et al. (1998) Plant Cell Rep 18:20-24; PCT Application WO 00/39313, the entire disclosures of which are hereby incorporated by reference).

A chimeric nucleic sequence construct is inserted into a plastid expression cassette including a promoter capable of expressing the construct in plant plastids. A particular promoter capable of expression in a plant plastid is, for example, a promoter isolated from the 5′ flanking region upstream of the coding region of a plastid gene, which may come from the same or a different species, and the native product of which is typically found in a majority of plastid types including those present in non-green tissues. Gene expression in plastids differs from nuclear gene expression and is related to gene expression in prokaryotes (Stern et al. (1997) Trends in Plant Sci 2:308-315, the entire disclosure of which is hereby incorporated by reference).

Plastid promoters generally contain the −35 and −10 elements typical of prokaryotic promoters, and some plastid promoters called PEP (plastid-encoded RNA polymerase) promoters are recognized by an E. coli-like RNA polymerase mostly encoded in the plastid genome, while other plastid promoters called NEP promoters are recognized by a nuclear-encoded RNA polymerase. Both types of plastid promoters are suitable for use herein. Examples of plastid promoters include promoters of clpP genes such as the tobacco clpP gene promoter (WO 97/06250, the entire disclosure of which is hereby incorporated by reference) and the Arabidopsis cipP gene promoter (U.S. application Ser. No. 09/038,878, the entire disclosure of which is hereby incorporated by reference). Another promoter capable of driving expression of a chimeric nucleic acid construct in plant plastids comes from the regulatory region of the plastid 16S ribosomal RNA operon (Harris et al., (1994) Microbiol Rev 58:700-754; Shinozaki et al. (1986) EMBO J 5:2043-2049, the entire disclosures of both of which are hereby incorporated by reference). Other examples of promoters capable of driving expression of a nucleic acid construct in plant plastids include a psbA promoter or am rbcL promoter. A plastid expression cassette preferably further includes a plastid gene 3′ untranslated sequence (3′ UTR) operatively linked to a chimeric nucleic acid construct of the present invention. The role of untranslated sequences is preferably to direct the 3′ processing of the transcribed RNA rather than termination of transcription. An exemplary 3′ UTR is a plastid rps16 gene 3′ untranslated sequence, or the Arabidopsis plastid psbA gene 3′ untranslated sequence. In a further embodiment, a plastid expression cassette includes a poly-G tract instead of a 3′ untranslated sequence. A plastid expression cassette also preferably further includes a 5′ untranslated sequence (5′ UTR) functional in plant plastids, operatively linked to a chimeric nucleic acid construct provided herein.

A plastid expression cassette is contained in a plastid transformation vector, which preferably further includes flanking regions for integration into the plastid genome by homologous recombination. The plastid transformation vector may optionally include at least one plastid origin of replication. The present invention also encompasses a plant plastid transformed with such a plastid transformation vector, wherein the chimeric nucleic acid construct is expressible in the plant plastid. Also encompassed herein is a plant or plant cell, including the progeny thereof, including this plant plastid. In a particular embodiment, the plant or plant cell, including the progeny thereof, is homoplasmic for transgenic plastids.

Other promoters capable of driving expression of a chimeric nucleic acid construct in plant plastids include transactivator-regulated promoters, preferably heterologous with respect to the plant or to the subcellular organelle or component of the plant cell in which expression is effected. In these cases, the DNA molecule encoding the transactivator is inserted into an appropriate nuclear expression cassette which is transformed into the plant nuclear DNA. The transactivator is targeted to plastids using a plastid transit peptide. The transactivator and the transactivator-driven DNA molecule are brought together either by crossing a selected plastid-transformed line with and a transgenic line containing a DNA molecule encoding the transactivator supplemented with a plastid-targeting sequence and operably linked to a nuclear promoter, or by directly transforming a plastid transformation vector containing the desired DNA molecule into a transgenic line containing a chimeric nucleic acid construct encoding the transactivator supplemented with a plastid-targeting sequence operably linked to a nuclear promoter. If the nuclear promoter is an inducible promoter, in particular a chemically inducible embodiment, expression of the chimeric nucleic acid construct in the plastids of plants is activated by foliar application of a chemical inducer. Such an inducible transactivator-mediated plastid expression system is preferably tightly regulatable, with no detectable expression prior to induction and exceptionally high expression and accumulation of protein following induction.

A particular transactivator is, for example, viral RNA polymerase. Particular promoters of this type are promoters recognized by a single sub-unit RNA polymerase, such as the T7 gene 10 promoter, which is recognized by the bacteriophage T7 DNA-dependent RNA polymerase. The gene encoding the T7 polymerase is preferably transformed into the nuclear genome and the T7 polymerase is targeted to the plastids using a plastid transit peptide. Promoters suitable for nuclear expression of a gene, for example a gene encoding a viral RNA polymerase such as the T7 polymerase, are described above and elsewhere in this application. Expression of chimeric nucleic acid constructs in plastids can be constitutive or can be inducible, and such plastid expression can be also organ- or tissue-specific. Examples of various expression systems are extensively described in WO 98/11235, the entire disclosure of which is hereby incorporated by reference. Thus, in one aspect, the present invention utilizes coupled expression in the nuclear genome of a chloroplast-targeted phage T7 RNA polymerase under the control of the chemically inducible PR-1a promoter, for example of the PR-1 promoter of tobacco, operably linked with a chloroplast reporter transgene regulated by T7 gene 10 promoter/terminator sequences, for example as described in as in U.S. Pat. No. 5,614,395 the entire disclosure of which is hereby incorporated by reference. In another embodiment, when plastid transformants homoplasmic for the maternally inherited TR or NTR genes are pollinated by lines expressing the T7 polymerase in the nucleus, F1 plants are obtained that carry both transgene constructs but do not express them until synthesis of large amounts of enzymatically active protein in the plastids is triggered by foliar application of the PR-1a inducer compound benzo(1,2,3)thiadiazole-7-carbothioic acid S-methyl ester (BTH).

In a particular embodiment, two or more genes, for example TR and NTR genes, are transcribed from the plastid genome from a single promoter in an operon-like polycistronic gene. In one embodiment, the operon-like polycistronic gene includes an intervening DNA sequence between two genes in the operon-like polycistronic gene. In a particular embodiment, the intervening DNA sequence is not present in the plastid genome to avoid homologous recombination with plastid sequences. In another embodiment, the DNA sequence is derived from the 5′ untranslated (UTR) region of a non-eukaryotic gene, preferably from a viral 5′UTR, preferably from a 5′UTR derived from a bacterial phage, such as a T7, T3 or SP6 phage. In one embodiment, a portion of the DNA sequence may be modified to prevent the formation of RNA secondary structures in an RNA transcript of the operon-like polycistronic gene, for example between the DNA sequence and the RBS of the downstream gene. Such secondary structures may inhibit or repress the expression of the downstream gene, particularly the initiation of translation. Such RNA secondary structures are predicted by determining their melting temperatures using computer models and programs such a the “mfold” program version 3 (available from Zuker and Turner, Washington University School of Medicine, St-Louis, Mo.) and other methods known to one skilled in the art.

The presence of the intervening DNA sequence in the operon-like polycistronic gene increases the accessibility of the RBS of the downstream gene, thus resulting in higher rates of expression. Such strategy is applicable to any two or more genes to be transcribed from the plastid genome from a single promoter in an operon-like chimeric heteromultimeric gene.

Following transformation the cells are grown, typically in a selective medium allowing the identification of transformants. Cells may be harvested in accordance with methodologies known to the art. In order to associate the oil bodies with the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, and a first and/or second thioredoxin-related protein, the integrity of cells may be disrupted using any physical, chemical or biological methodology capable of disrupting the cells' integrity. These methodologies are generally cell-type dependent and known to the skilled artisan. Where plants are employed they may be regenerated into mature plants using plant tissue culture techniques generally known to the skilled artisan. Seeds may be harvested from mature transformed plants and used to propagate the plant line. Plants may also be crossed and in this manner, contemplated herein is the breedig of cells lines and transgenic plants that vary in genetic background. It is also possible to cross a plant line comprising the first recombinant polypeptide with a plant line comprising the second recombinant polypeptide. Accordingly, also provided herein are methods of producing in a plant a recombinant multimeric-protein-complex, said method comprising:

(a) preparing a first plant comprising cells, said cells comprising oil bodies and a first recombinant polypeptide, such as a redox protein (e.g., a thioredoxin-related protein, and the like) or an immunoglobulin-polypeptide-chain, wherein said first recombinant polypeptide is capable of associating with said oil bodies through an oil-body-targeting-protein;

(b) preparing a second plant comprising cells, said cells comprising oil bodies and a second recombinant polypeptide, such as a second redox protein (e.g., a thioredoxin-related protein, and the like) or a second immunoglobulin-polypeptide-chain; and

(c) sexually crossing said first plant with said second plant to produce a progeny plant comprising cells, said cells comprising oil bodies, wherein said oil bodies are capable of associating with said first recombinant polypeptide, and said first recombinant recombinant polypeptide is capable of associating with said second recombinant polypeptide to form said recombinant multimeric-protein-complex.

The second recombinant polypeptide may also associate with the oil bodies. Accordingly, also provided herein are methods of producing in a plant a recombinant multimeric-protein-complex, said method comprising:

(a) preparing a first plant comprising cells, said cells comprising oil bodies and a first recombinant polypeptide, such as a redox (or thioredoxin-related) protein or immunoglobulin-polypeptide-chain, wherein said first recombinant polypeptide is capable of associating with said oil bodies through an oil-body-targeting-protein;

(b) preparing a second plant comprising cells, said cells comprising oil bodies and a second recombinant polypeptide, such as a second redox (thioredoxin-related) protein or a second immunoglobulin-polypeptide-chain, wherein said second recombinant polypeptide is capable of associating with said oil bodies through an oil body targeting protein; and

(c) sexually crossing said first plant with said second plant to produce a progeny plant comprising cells, said cells comprising oil bodies, wherein said oil bodies are capable of associating with said first recombinant polypeptide, and said first recombinant recombinant polypeptide is capable of associating with said second recombinant polypeptide to form said recombinant multimeric-protein-complex.

Isolation of Oil Bodies

The oil bodies provided herein may be obtained from any cell containing oil bodies, including any animal cell; plant cell; fungal cell; for example a yeast cell, algae cell; or bacterial cell. Any process suitable for the isolation oil bodies from cells may be used herein. Processes for the isolation of oil bodies from plant seed cells have been described in U.S. patents (U.S. Pat. Nos. 6,146,645 and 6,183,762) and the isolation of oil bodies from yeast cells has been described by Ting et al. (1997) J. Biol. Chem. 272: 3699-3706).

In certain embodiments, the oil bodies are obtained from a plant cell such as for example a pollen cell; a fruit cell; a spore cell; a nut cell; mesocarp cell; for example the mesocarp cells obtainable from olive (Olea europaea) or avocado (Persea americana); or a seed cell. In particular embodiments the oil bodies are obtained from a plant seed cell. The seeds can be obtained from a transgenic plant according to the present invention. In particular embodiments, a seed of a transgenic plant according to the present invention contains the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, or first and/or second thioredoxin-related proteins in a concentration of at least about 0.5% of total cellular seed protein. In further embodiments, a seed of a transgenic plant provided herein contains a recombinant polypeptide or multimeric-protein-complex in a concentration of at least about 0.5%, 0.6%, 0.7%, 0.8%, 0.9%, 1.0%, 1.25%, 1.5%, 1.75%, 2.0%, 2.25%, 2.5%, 3%, 4%, 5%, 6%, 7%, 8%, 9%, 10% or more, of total cellular seed protein. The upper limits of the recombinant polypeptide or multimeric-protein-complex concentration can be up to about 8%, 9%, 10%, 11%, 12%, 13%, 14%, 15%. Thus, the ranges at least about 0.5% up to about 15%; at least about 1.0% up to about 10%; and at least about 5% up to about 8% are amoung the various ranges contemplated herein.

Among the plant seeds useful in this regard are plant seeds obtainable from the group of plant species consisting of Brazil nut (Betholletia excelsa); castor (Riccinus communis); coconut (Cocus nucifera); coriander (Coriandrum sativum); cotton (Gossypium spp.); groundnut (Arachis hypogaea); jojoba (Simmondsia chinensis); linseed/flax (Linum usitatissimum); maize (Zea mays); mustard (Brassica spp. and Sinapis alba); oil palm (Elaeis guineeis); olive (Olea europaea); rapeseed (Brassica spp.); safflower (Carthamus tinctorius); soybean (Glycine max); squash (Cucurbita maxima); sunflower (Helianthus annuus); barley (Hordeum vulgare); wheat (Traeticum aestivum) and mixtures thereof. In a particular embodiment, oil bodies are obtainable from the seeds obtainable from safflower (Carthamus tinctorius).

In order to prepare oil bodies from plant seeds, plants are grown and allowed to set seed in accordance with common agricultural practices. Thus, the present invention also provides seeds comprising oil bodies, wherein said oil bodies further comprise invention multimeric-protein-complexes described herein. Upon harvesting the seed and, if necessary the removal of large insoluble materials such as stones or seed hulls, by for example sieving or rinsing, any process suitable for the isolation of oil bodies from seeds may be used herein. A typical process involves grinding of the seeds followed by an aqueous extraction process.

Seed grinding may be accomplished by any comminuting process resulting in a substantial disruption of the seed cell membrane and cell walls without compromising the structural integrity of the oil bodies present in the seed cell. Suitable grinding processes in this regard include mechanical pressing and milling of the seed. Wet milling processes such as decribed for cotton (Lawhon et al. (1977) J. Am. Oil Chem. Soc. 63: 533-534) and soybean (U.S. Pat. No. 3,971,856; Carter et al. (1974) J. Am. Oil Chem. Soc. 51: 137-141) are particularly useful in this regard. Suitable milling equipment capable of industrial scale seed milling include colloid mills, disc mills, pin mills, orbital mills, IKA mills and industrial scale homogenizers. The selection of the milling equipment will depend on the seed, which is selected, as well as the throughput requirement.

Solid contaminants such as seed hulls, fibrous materials, undissolved carbohydrates, proteins and other insoluble contaminants are subsequently preferably removed from the ground seed fraction using size exclusion based methodologies such as filtering or gravitational based methods such as a centrifugation based separation process. Centrifugation may be accomplished using for example a decantation centrifuge such as a HASCO 200 2-phase decantation centrifuge or an NX310B (Alpha Laval). Operating conditions are selected such that a substantial portion of the insoluble contaminants and sediments and may be separated from the soluble fraction.

Following the removal of insolubles the oil body fraction may be separated from the aqueous fraction. Gravitational based methods as well as size exclusion based technologies may be used. Gravitational based methods that may be used include centrifugation using for example a tubular bowl centrifuge such as a Sharples AS-16 or AS-46 (Alpha Laval), a disc stack centrifuge or a hydrocyclone, or separation of the phases under natural gravitation. Size exclusion methodologies that may be used include membrane ultra filtration and crossflow microfiltration.

Separation of solids and separation of the oil body phase from the aqueous phase may also be carried out concomitantly using gravity based separation methods or size exclusion based methods.

The oil body preparations obtained at this stage in the process are generally relatively crude and depending on the application of the oil bodies, it may be desirable to remove additional contaminants. Any process capable of removing additional seed contaminants may be used in this regard. Conveniently the removal of these contaminants from the oil body preparation may be accomplished by resuspending the oil body preparation in an aqueous phase and re-centrifuging the resuspended fraction, a process referred to herein as “washing the oil bodies”. The washing conditions selected may vary depending on the desired purity of the oil body fractions. For example where oil bodies are used in pharmaceutical compositions, generally a higher degree of purity may be desirable than when the oil bodies are used in food preparations. The oil bodies may be washed one or more times depending on the desired purity and the ionic strength, pH and temperature may all be varied. Analytical techniques may be used to monitor the removal of contaminants. For example SDS gel electrophoresis may be employed to monitor the removal of seed proteins.

The entire oil body isolation process may be performed in a batch wise fashion or continuous flow. In a particular embodiment, industrial scale continuous flow processes are utilized.

Through the application of these and similar techniques the skilled artisan is able to obtain oil bodies from any cell comprising oil bodies. The skilled artisan will recognize that generally the process will vary somewhat depending on the cell type that is selected. However, such variations may be made without departing from the scope and spirit of the present invention.

Association of the First and/or Second Recombinant Polypeptides, Multimeric-Protein-Complexes, Heteromultimeric-Protein-Complexes, Multimeric-Fusion-Proteins, Heteromultimeric-Fusion-Proteins, Immunoglobulins, Immunoglobulin-Polypeptide-Chains, Redox-Fusion-Polypeptides, the First and/or Second Thioredoxin-Related Proteins with Oil Bodies.

In accordance with the present invention, the oil bodies are associated with either the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, the first and/or second thioredoxin-related proteins through association with an oil-body-targeting-protein capable of association with these multimeric-protein-complexes and the oil bodies. As used herein the phrase “associating the oil bodies with the multimeric-protein-complex” means that the oil bodies are brought in proximity of the multimeric-protein-complexes in a manner that allows the association of the oil bodies with either the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, or the first and/or second thioredoxin-related proteins. The association of the oil bodies with the multimeric-protein-complexes is accomplished by association of the oil-body-targeting-protein with both the oil body and with the multimeric-protein-complex. In particular embodiments, the cells expressing the multimeric-protein-complex associate with the oil bodies that are obtainable from these same cells, which permits the convenient production and isolation of the multimeric-protein-complex, including the first and/or second recombinant polypeptides, heteromultimeric-protein-complexes, multimeric-fusion-proteins, heteromultimeric-fusion-proteins, immunoglobulins, immunoglobulin-polypeptide-chains, redox-fusion-polypeptides, or the first and/or second thioredoxin-related proteins, in an oil body-comprising host cell system. Accordingly, in one embodiment, the association of the oil body with the multimeric-protein-complex is accomplished intracellularly during the growth of the cell. For example, a redox fusion polypeptide may be fused to an oil-body-protein and the chimeric protein may be expressed in oil body-containing plant seeds. Isolation of the oil bodies from the seeds in this case results in isolation of oil bodies comprising either the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, or the first and/or second thioredoxin-related proteins. In another embodiment, in which the multimeric-protein-complex associates with oil bodies obtainable from the same cells in which the complex is produced, the association of the oil bodies with the multimeric-protein-complex is accomplished upon disrupting the cell's integrity.

For example, the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, or the first and/or second thioredoxin-related proteins may be expressed in such a manner that it is targeted to the endomembrane system of the seed cells. Oil bodies present in the same seed cells comprising an oil-body-targeting-protein capable of association with these multimeric-protein-complexes, for example an oleosin linked to a single chain antibody capable of association with a recombinant polypeptide or multimeric-protein-complex, may then associate with the recombinant polypeptide or multimeric-protein-complex upon grinding of the seed.

In accordance with this embodiment, plant seed cells comprising a light and heavy chain of an immunoglobulin targeted to the plant apoplast can be prepared. These particular seed cells are prepared to further comprise oil bodies associated with an oil-body-targeting-protein capable of association with the immunoglobulin, such as for example, an oleosin-protein A fusion protein, and the like. Upon grinding of the seed, the oil bodies comprising protein A associate with the immunoglobulin through binding.

In yet another embodiment, the oil bodies used to associate with the multimeric-protein-complex are obtained from a cellular source different from the cell comprising the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, or the first and/or second thioredoxin-related proteins, such as from a separate plant line. For example, oil bodies associated with protein A may be prepared from one plant line. These oil bodies may then be mixed with ground seeds comprising an apoplastically expressed light and heavy chain constituting an immunoglobulin. Alternatively, a plant line comprising oil bodies associated with protein A may be crossed with a plant line comprising an immunoglobulin.

The first recombinant polypeptide, second recombinant polypeptide and oil-body-targeting-protein may also be prepared in separate cellular compartments. Association of the first polypeptide, second polypeptide, and oil body then may occur upon disruption of the cell's integrity. For exmple, various mechanisms for targeting gene products are known to exist in plants, and the sequences controlling the functioning of these mechanisms have been characterized in some detail. For example, the targeting of gene products to the chloroplast is controlled by a transit sequence found at the amino terminal end of various proteins which is cleaved during chloroplast import to yield the mature protein (Comai et al. (1988) J Biol Chem 263: 15104-15109). Other gene products are localized to other organelles such as the mitochondrion and the peroxisome (Unger et al. (1989) Plant Mol Biol 13:411-418). The cDNAs encoding these products can be manipulated to target heterologous gene products to these organelles. In addition, sequences have been characterized which cause the targeting of gene products to other cell compartments.

Amino terminal sequences are responsible for targeting to the ER, the apoplast, and extracellular secretion from aleurone cells (Koehler & Ho (1990) Plant Cell 2:769-783). Additionally, amino terminal sequences in conjunction with carboxy terminal sequences are responsible for vacuolar targeting of gene products (Shinshi et al., (1990) Plant Mol Biol 14:357-368). By the fusion of the appropriate targeting sequences described above to transgene sequences of interest it is possible to direct the transgene product to the desired organelle or cell compartment.

As hereinbefore mentioned, the redox protein obtained using the methods provided herein is enzymatically active while associated with the oil body. Preferably the redox protein is at least 5 times more active when produced as a redox fusion polypeptide with a second redox protein relative to its production in association with an oil body as a non-fusion polypeptide (i.e. without the second redox protein). More preferably the redox protein is at least 10 times more active when produced as a redox fusion polypeptide.

The activity of the redox fusion polypeptide may be determined in accordance with methodologies generally known to the art (see for example: Johnson et al (1984) J. of Bact. Vol. 158 3:1061-1069) and may be optimized by for example the addition of detergents, including ionic and non-ionic detergents.

Formulation of Oil Bodies

In accordance with a particular embodiment, the oil bodies comprising the first and/or second recombinant polypeptides, multimeric-protein-complexes, heteromultimeric-protein-complexes, multimeric-fusion-proteins, heteromultimeric-fusion-proteins, immunoglobulins, immunoglobulin-polypeptide-chains, redox-fusion-polypeptides, or the first and/or second thioredoxin-related proteins, are preferably formulated into an emulsion. The emulsion is preferably used in the preparation of a pharmaceutical composition, personal care or a food product. In emulsified form, the oil body offers certain desirable properties, such as for example excellent compatibility with the human skin.

It particular embodiments, the oil body formulation is stabilized so that a final product may be obtained which may be stored and preserved for longer periods of time. As used herein, the term “stabilized oil body preparation” refers to an oil body preparation that is prepared so that the formulation does not undergo undesirable physical or chemical alterations when the oil body preparation is stored. The stabilization requirements may vary depending on the final product. For example personal care products are preferably stable for at least one year at room temperature while additionally being able to withstand short temperature fluctuations. Pharmaceutical formulations may in some cases be less stable as they may be stored at lower temperatures thereby preventing the occurrence of undesirable reactions.

In general, stabilization techniques that may be used herein include any and all methods for the preservation of biological material including the addition of chemical agents, temperature modulation based methodologies, radiation-based technologies and combinations thereof. In particular embodiments small amounts of stabilizing chemical agents are mixed with the oil body formulation to achieve stabilization. These chemical agents include inter alia preservatives, antioxidants, acids, salts, bases, viscosity modifying agents, emulsifiers, gelling agents and mixtures thereof and may all be used to stabilize the oil body preparation. In view of the presence of the redox fusion polypeptide the stabilizing agent is generally selected to be compatible with and resulting in good enzymatic function of the redox fusion polypeptide.

Diagnostic parameters to assess the stability of the oil body preparation may be as desired and include all parameters indicative of undesirable qualitative or quantitative changes with respect to chemical or physical stability. Typical parameters to assess the oil body preparation over time include color, odor, viscosity, texture, pH and microbial growth, and enzymatic activity.

In particular embodiments, the oil body formulation is stabilized prior to the addition of further ingredients that may be used to prepare the final product. Howevera, in other embodiments, it is nevertheless possible to formulate the final formulation using non-stabilized oil bodies and stabilize the final formulation. The final preparations may be obtained using one or more additional ingredients and any formulation process suitable for the preparation of a formulation comprising oil bodies. Ingredients and processes employed will generally vary depending on the desired use of the final product, will be art recognized and may be as desired. Ingredients and processes that may be used herein include those described in U.S. patents (U.S. Pat. Nos. 6,146,645 and 6,183,762) which are incorporated by reference herein.

In particular embodiments, the redox fusion polypeptide comprises a thioredoxin and a thioredoxin-reductase. Accordingly, provided herein are oil bodies comprising a thioredoxin/thioredoxin-reductase fusion polypeptide. Also provided herein is a formulation containing oil bodies comprising a thioredoxin/thioredoxin-reductase fusion capable of treating or protecting a target against oxidative stress. The stress of the target is treated or prevented by contacting the target with the formulation. The target may be any substance susceptible to oxidative stress, including any molecule, molecular complex, cell, tissue or organ.

In another embodiment, provided herein is a formulation containing oil bodies comprising a thioredoxin/thioredoxin-reductase fusion capable of chemically reducing a target. Contacting the target with the formulation reduces the target. The target may be any substance susceptible to reduction, including any molecule or molecular complex. Particularly susceptible targets in this regard are the disulfide bonds present in proteins.

The oil bodies comprising thioredoxin/thioredoxin-reductase may be used to prepare formulations used to reduce the allergenicity of food or increase the digestibility of food. Preferably, the method of reducing the food allergenicity is practiced by mixing the thioredoxin/thioredoxin-reductase comprising oil bodies with food or food ingredients selected from a variety of sources including for example wheat flour, wheat dough, milk, cheese, soya, yogurt and ice cream. The thioredoxin/thioredoxin-reductase comprising oil bodies may also be used to increase the digestibility of milk as well as other disulfide containing proteins (Jiao, J. et al. (1992) J. Agric. Food Chem 40: 2333-2336). Further food applications include the use of the oil thioredoxin/thioredoxin-reductase comprising oil bodies as a food additive to enhance dough strength and bread quality properties (Wong et al., (1993) J. Cereal Chem. 70: 113-114; Kobrehel et al. (1994) Gluten Proteins: Association of Cereal Research; Detmold, Germany).

Also provided herein are pharmaceutical compositions comprising, in a pharmaceutically active carrier: oil bodies comprising a thioredoxin/thioredoxin-reductase; oil bodies comprising multimeric-protein-complexes, such as heteromultimeric-protein-complexes; isolated thioredoxin/thioredoxin-reductase fusion proteins; or isolated multimeric-protein-complexes. These pharmaceutical compositions may be used for the treatment of reperfusion injury (Aota et al. (1996) J. Cardiov. Pharmacol. (1996) 27: 727-732), cataracts (U.S. patent U.S. Pat. No. 4,771,036), chronic obstructive pulmonary disease (COPD) (MacNee et al. (1999) Am. J. Respir. Crit. Care Med. 160:S58-S65), diabetes (Hotta et al. J. Exp. Med. 188: 1445-1451), envenomation (PCT Patent Application 99/20122; U.S. Pat. No. 5,792,506), bronchiopulmonary disease (MacNee (2000) Chest 11 7:3035-3175); malignancies (PCT Patent Application 91/04320) and the alleviation of the allergenic potential of airborne, for example pollen-derived, and contact allergens (PCT Patent Application 00/44781). Other diseases or conditions that may be treated with the pharmaceutical compositions provided herein include: psoriasis, wound healing, sepsis, GI bleeding, intestinal bowel disease (IBD), ulcers, transplantation, GERD (gastro esophageal reflux disease).

In another embodiment, the pharmaceutical compositions provided herein, particularly those comprising one or more redox proteins alone or in combination with oil bodies, can be used in the treatment of inflammatory and viral diseases by reductively inactivating phospholipase A2, one of the contributing factors in inflammatory diseases. Additionally, the redox fusion polypeptide system has been found to function as a self-defense mechanism in response to environmental stimuli, including oxidative stress caused by UV-generated free radicals. Consequently, redox proteins, e.g., oleosin-thioredoxin, oleosin-thioredoxin-reductase, the various redox fusion polypeptides described herein, provide beneficial effects in certain skin conditions such as psoriasis, skin cancer, dandruff, diaper rash, dermatitis, acne, sun damage, aging, inflammation, and the like.

In another embodiment, oil-body-thioredoxin-related fusion proteins, e.g., oleosin-Thioredoxin-reductase, can also be used as a venom antidote. Many animal venoms and other toxins contain disulfide bonds, including all snake venom neurotoxins, some bacterial neurotoxins including tetanus and botulinum A, bee venom phospholipase A₂, and scorpion venom. In a further embodiment, the redox protein related pharmaceutical compositions provided herein can be used to inactivate venom toxins by reduction of disulfide bonds. A method of treating an individual suffering from the effects of a venom or toxin can include the step of administering an effective dose of a pharmaceutical composition, in a pharmaceutically effective carrier in an amount sufficient to relieve or reverse the effects of the venom toxin on the individual.

The pharmaceutical compositions provided herein are preferably formulated for single dosage administration. The concentrations of the compounds in the formulations are effective for delivery of an amount, upon administration, that is effective for the intended treatment. Typically, the compositions are formulated for single dosage administration. To formulate a composition, the weight fraction of a compound or mixture thereof is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, may also be suitable as pharmaceutically acceptable carriers. These may be prepared according to methods known to those skilled in the art. For example, liposome formulations may be prepared as described in U.S. Pat. No. 4,522,811.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo systems, such as the assays provided herein.

The concentration of active compound in the drug composition will depend on absorption, inactivation and excretion rates of the active compound, the physicochemical characteristics of the compound, the dosage schedule, and amount administered as well as other factors known to those of skill in the art.

Typically a therapeutically effective dosage is contemplated. The amounts administered may be on the order of 0.001 to 1 mg/ml, preferably about 0.005-0.05 mg/ml, more preferably about 0.01 mg/ml, of blood volume. Pharmaceutical dosage unit forms are prepared to provide from about 1 mg to about 1000 mg and preferably from about 10 to about 500 mg, more preferably about 25-75 mg of the essential active ingredient or a combination of essential ingredients per dosage unit form. The precise dosage can be empirically determined.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of the claimed compositions and combinations containing them.

Preferred pharmaceutically acceptable derivatives include acids, salts, esters, hydrates, solvates and prodrug forms. The derivative is typically selected such that its pharmacokinetic properties are superior to the corresponding neutral compound.

Thus, effective concentrations or amounts of one or more of the compounds provided herein or pharmaceutically acceptable derivatives thereof are mixed with a suitable pharmaceutical carrier or vehicle for systemic, topical or local administration to form pharmaceutical compositions. Compounds are included in an amount effective for ameliorating or treating the disorder for which treatment is contemplated. The concentration of active compound in the composition will depend on absorption, inactivation, excretion rates of the active compound, the dosage schedule, amount administered, particular formulation as well as other factors known to those of skill in the art.

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as Tween®, or dissolution in aqueous sodium bicarbonate. Derivatives of the compounds, such as prodrugs of the compounds may also be used in formulating effective pharmaceutical compositions. For ophthalmic indications, the compositions are formulated in an ophthalmically acceptable carrier. For the ophthalmic uses herein, local administration, either by topical administration or by injection is preferred. Time release formulations are also desirable. Typically, the compositions are formulated for single dosage administration, so that a single dose administers an effective amount.

Upon mixing or addition of the compound with the vehicle, the resulting mixture may be a solution, suspension, emulsion or other composition. The form of the resulting mixture depends upon a number of factors, including the intended mode of administration and the solubility of the compound in the selected carrier or vehicle. If necessary, pharmaceutically acceptable salts or other derivatives of the compounds are prepared.

The compound is included in the pharmaceutically acceptable carrier in an amount sufficient to exert a therapeutically useful effect in the absence of undesirable side effects on the patient treated. It is understood that number and degree of side effects depends upon the condition for which the compounds are administered. For example, certain toxic and undesirable side effects are tolerated when treating life-threatening illnesses that would not be tolerated when treating disorders of lesser consequence.

The compounds can also be mixed with other active materials, that do not impair the desired action, or with materials that supplement the desired action known to those of skill in the art. The formulations of the compounds and agents for use herein include those suitable for oral, rectal, topical, inhalational, buccal (e.g., sublingual), parenteral (e.g., subcutaneous, intramuscular, intradermal, or intravenous), transdermal administration or any route. The most suitable route in any given case will depend on the nature and severity of the condition being treated and on the nature of the particular active compound which is being used. The formulations are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutically acceptable carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, or carboxymethyl-cellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Methods of preparing such dosage forms are known, or will be apparent, to those skilled in this art (see, e.g., Remington's Pharmaceutical Sciences, Mack Publishing Company, Easton, Pa., 15th Edition, 1975). The composition or formulation to be administered will contain a quantity of the active compound in an amount sufficient to alleviate the symptoms of the treated subject.

Dosage forms or compositions containing active ingredient in the range of 0.005% to 100% with the balance made up from non-toxic carrier may be prepared. For oral administration, the pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

The pharmaceutical preparation may also be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin or to the eye preferably take the form of an ointment, cream, lotion, paste, gel, spray, aerosol and oil. Carriers which may be used include vaseline, lanoline, polyethylene glycols, alcohols, and combinations of two or more thereof. The topical formulations may further advantageously contain 0.05 to 15 percent by weight of thickeners selected from among hydroxypropyl methyl cellulose, methyl cellulose, polyvinylpyrrolidone, polyvinyl alcohol, poly (alkylene glycols), poly/hydroxyalkyl, (meth)acrylates or poly(meth)acrylamides. A topical formulation is often applied by instillation or as an ointment into the conjunctival sac. It can also be used for irrigation or lubrication of the eye, facial sinuses, and external auditory meatus. It may also be injected into the anterior eye chamber and other places. The topical formulations in the liquid state may be also present in a hydrophilic three-dimensional polymer matrix in the form of a strip, contact lens, and the like from which the active components are released.

For administration by inhalation, the compounds for use herein can be delivered in the form of an aerosol spray presentation from pressurized packs or a nebulizer, with the use of a suitable propellant, e.g., dichlorodi-fluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin, for use in an inhaler or insufflator may be formulated containing a powder mix of the compound and a suitable powder base such as lactose or starch.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

The compounds may be formulated for parenteral administration by injection, e.g., by bolus injection or continuous infusion. Formulations for injection may be presented in unit dosage form, e.g., in ampules or in multi-dose containers, with an added preservative. The compositions may be suspensions, solutions or emulsions in oily or aqueous vehicles, and may contain formulatory agents such as suspending, stabilizing and/or dispersing agents. Alternatively, the active ingredient may be in powder form for reconstitution with a suitable vehicle, e.g., sterile pyrogen-free water or other solvents, before use.

Formulations suitable for transdermal administration may be presented as discrete patches adapted to remain in intimate contact with the epidermis of the recipient for a prolonged period of time. Such patches suitably contain the active compound as an optionally buffered aqueous solution of, for example, 0.1 to 0.2 M concentration with respect to the active compound. Formulations suitable for transdermal administration may also be delivered by iontophoresis (see, e.g., Pharmaceutical Research 3 (6), 318 (1986)) and typically take the form of an optionally buffered aqueous solution of the active compound.

The pharmaceutical compositions may also be administered by controlled release means and/or delivery devices (see, e.g., in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566).

Desirable blood levels may be maintained by a continuous infusion of the active agent as ascertained by plasma levels. It should be noted that the attending physician would know how to and when to terminate, interrupt or adjust therapy to lower dosage due to toxicity, or bone marrow, liver or kidney dysfunctions. Conversely, the attending physician would also know how to and when to adjust treatment to higher levels if the clinical response is not adequate (precluding toxic side effects).

The efficacy and/or toxicity of the pharmaceutical compositions provided herein, alone or in combination with other agents can also be assessed by the methods known in the art (See generally, O'Reilly, Investigational New Drugs, 15:5-13 (1997)).

The active compounds or pharmaceutically acceptable derivatives may be prepared with carriers that protect the compound against rapid elimination from the body, such as time release formulations or coatings.

Kits containing the compositions and/or the combinations with instructions for administration thereof are provided. The kit may further include a needle or syringe, preferably packaged in sterile form, for injecting the complex, and/or a packaged alcohol pad. Instructions are optionally included for administration of the active agent by a clinician or by the patient.

Finally, the pharmaceutical compositions provided herein containing any of the preceding agents may be packaged as articles of manufacture containing packaging material, a compound or suitable derivative thereof provided herein, which is effective for treatment of a diseases or disorders contemplated herein, within the packaging material, and a label that indicates that the compound or a suitable derivative thereof is for treating the diseases or disorders contemplated herein. The label can optionally include the disorders for which the therapy is warranted.

Also provided herein are personal care formulations containing oil bodies comprising a thioredoxin/thioredoxin-reductase fusion polypeptide. Personal care products comprising thioredoxin and thioredoxin-reductase are disclosed in for example Japanese Patent Applications JP9012471A2, JP103743A2, and JP1129785A2 Personal care formulations that may be prepared in accordance with the present invention include formulations capable of improving the physical appearance of skin exposed to detrimental environmental stimuli resulting in oxidative stress for example oxidative stress caused by UV-generated free-radicals. The oil bodies comprising thioredoxin/thioredoxin-reductase may also be used to prepare hair care products as described in U.S. Pat. Nos. 4,935,231 and 4,973,475 (incorporated herein by reference in their entirety).

The following examples are included for illustrative purposes only and are not intended to limit the scope of the invention.

### EXAMPLE 1

Isolation of Thioredoxin and NADPH Thioredoxin-Reductase Genes

An Arabidopsis silique cDNA library CD4-12 was obtained from the Arabidopsis Biological Resource Centre (ABRC, http://aims.cps.msu.edu) Arabidopsis stock centre and used as a template for the isolation of the thioredoxin h (Trxh) and thioredoxin-reductase genes from Arabidopsis. For the isolation of the Trxh gene the following primers were synthesized:

GVR833: 5′TACCATGGCTTCGGAAGAAGGA 3′ (SEQ ID NO:1)

The sequence identical to the 5′ end of the Trxh gene as published in Rivera-Madrid et al, (1993) Plant Physiol 102: 327-328, is indicated in bold. Underlined is an NcoI restriction site to facilitate cloning. GVR834:

5′GAAAGCTTAAGCCAAGTGTTTG 3′ (SEQ ID NO:2)

The sequence complementary to the 3′ end of the Trxh gene as published in Rivera-Madrid et al, (1993) Plant Physiol 102: 327-328, is indicated in bold. Underlined is an Hindlul restriction site to facilitate cloning.

A Polymerase Chain Reaction (PCR) was carried out using GVR833 and GVR834 as primers and the cDNA library CD4-12 as a template. The resulted PCR fragment was isolated, cloned into pBluescript and sequenced. The isolated sequence encoding Trxh was identical to the published Trxh gene sequence (Rivera-Madrid et al, (1993) Plant Physiol 102: 327-328). The pBluescript vector containing the Trxh gene is called pSBS2500.

For the isolation of the thioredoxin-reductase gene the following primers were synthesized:

GVR836: 5′ GGCCAGCACACTACCATGAATGGTCTCGAAACTCAC 3′ (SEQ ID NO:3). The sequence identical to the 5′ end of the thioredoxin-reductase gene as published (Jacquot et al, J Mol Biol. (1994) 235 (4):1357-63), is indicated in bold).

GVR837: 5′ TTAAGCTTCAATCACTCTTACCTTGCTG 3′ (SEQ ID NO:4).

A Polymerase Chain Reaction (PCR) was carried out using GVR836 and GVR837 as primers and the cDNA library CD4-12 as a template. The resulted PCR fragment was isolated, cloned into pBluescript and sequenced. The pBluescript vector containing the thioredoxin-reductase gene is called pSBS2502.

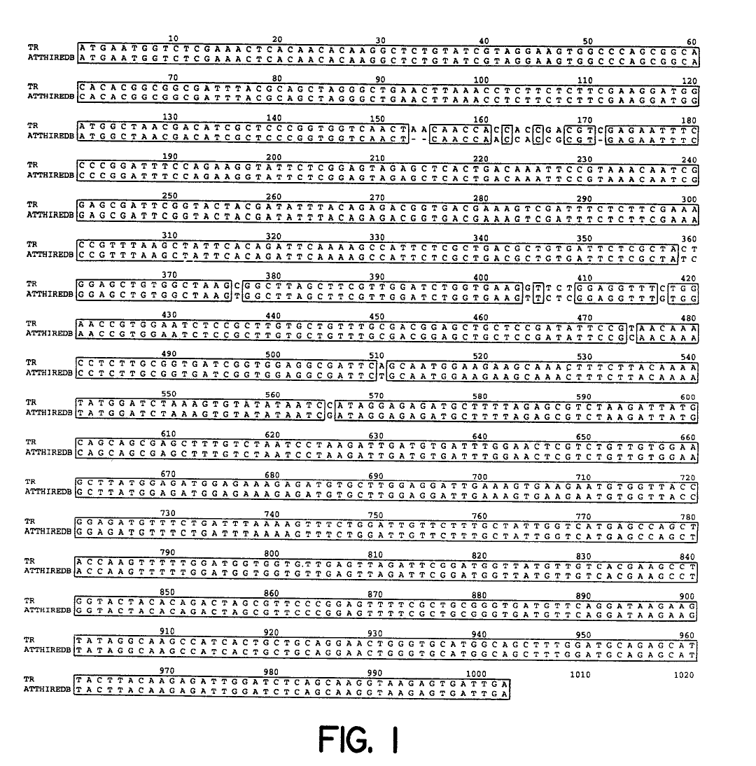

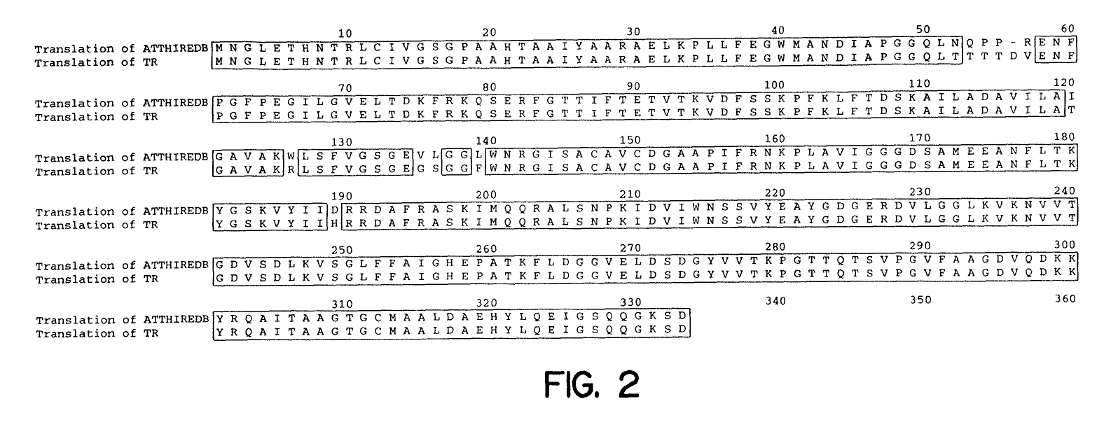

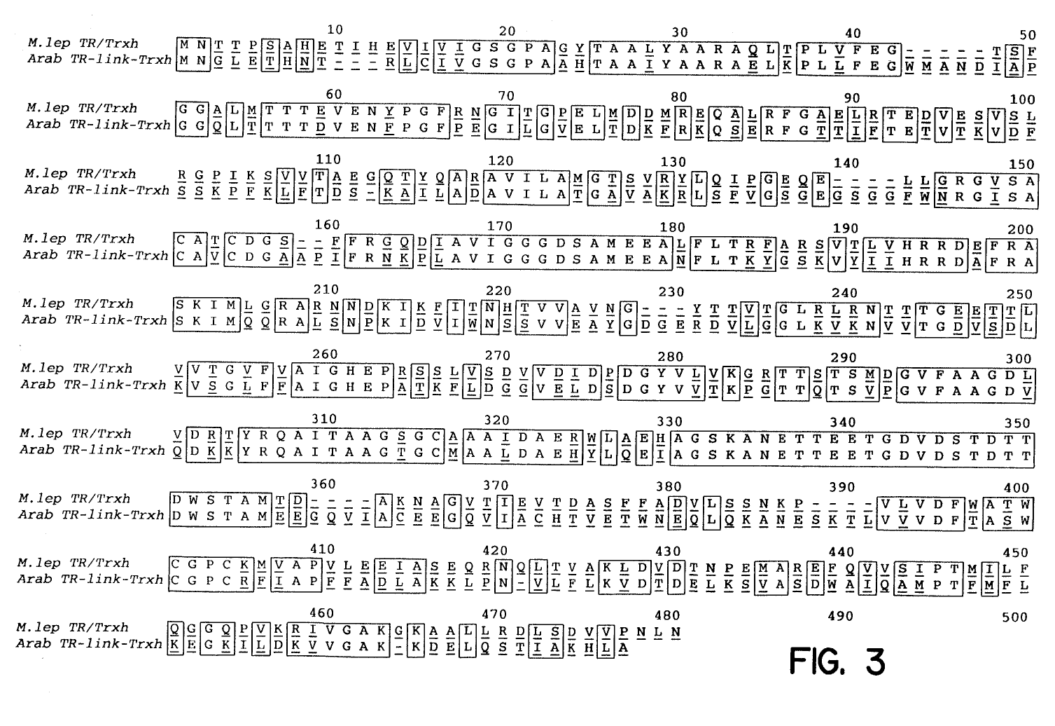

A total of three clones were sequenced, the sequence of each of the three clones were identical to each other. However, as depicted in FIG. 1 this sequence indicated several nucleotide differences compared to the published thioredoxin-reductase gene sequence published (Jacquot et al, J Mol Biol. (1994) 235 (4):1357-63.). The complete coding sequence and its deduced amino acid sequence is shown in SEQ ID NO:10. As a result of the nucleotide differences between the published sequence and the sequence isolated in Example 1, several amino acid changes are also predicted. A comparison of the deduced amino acid sequence of the published NADPH thioredoxin-reductase sequence thioredoxin-reductase (ATTHIREDB, Jacquot et al, J Mol Biol. (1994) 235 (4):1357-63.) with the sequence isolated in Example 1 (TR) is shown in FIG. 3.

### EXAMPLE 2

Construction of Plant Expression Vectors.

Expression vectors were constructed to allow for the seed specific over-expression of thioredoxin and NADPH thioredoxin-reductase in seeds. Vectors were constructed to allow for over-expression in its natural subcellular location and for accumulation on oilbodies.

Construction of Plant Transformation Vector pSBS2520.

The Arabidopsis thioredoxin h gene as described in example 1 was placed under the regulatory control of the phaseolin promoter and the phaseolin terminator derived from the common bean Phaseolus vulgaris (Slightom et al (1983) Proc. Natl Acad Sc USA 80: 1897-1901; Sengupta-Gopalan et al., (1985) PNAS USA 82: 3320-3324)). A gene splicing by overlap extension technique (Horton et al (1989) 15: 61-68) was used to fuse the phaseolin promoter to the Trxh gene. Standard molecular biology laboratory techniques (see eg: Sambrook et al. (1990) Molecular Cloning, 2^{nd} ed. Cold Spring Harbor Press) were used to furnish the phaseolin promoter and terminator with Pst I and HindIII/KpnI sites respectively (see SEQ ID NO:14). Standard molecular biology laboratory techniques were also used to place the phaseolin terminator dowstream from the Trxh gene. The PstI-phaseolin promoter- Trxh-phaseolin terminator-KpnI insert sequence was cloned into the PstI-KpnI sites of pSBS3000 (pSBS3000 is a derivative from the Agrobacterium binary plasmid pPZP221 (Hajdukiewicz et al., 1994, Plant Molec. Biol. 25: 989-994). In pSBS3000, the CaMV35S promoter-gentamycin resistance gene-CAMV 35S terminator of pPZP221 was replaced with parsley ubiquitin promoter-phosphinothricin acetyl transferase gene-parsley ubiquitin termination sequence to confer resistance to the herbicide glufosinate ammonium.) The resulting plasmid is called pSBS2520. The sequence of the phaseolin promoter-Arabidopsis Trxh-phaseolin terminator sequence is shown in SEQ ID NO:14.

Construction of Plant Transformation Vector pSBS2510.

The 3′ coding sequence of an Arabidopsis oleosin gene (van Rooijen et al (1992) Plant Mol. Biol.18: 1177-1179) was altered to contain an NcoI site. The NcoI-HindIII fragment from vector pSBS2500 (Example 1) containing the Trxh was ligated to the coding sequence of this Arabidopsis oleosin utilizing this NcoI restriction site. A gene splicing by overlap extension technique (Horton et al (1989) 15: 61-68) was used to fuse the phaseolin promoter (Slightom et al (1983) Proc. Natl Acad Sc USA 80:1897-1901; Sengupta-Gopalan et al., (1985) PNAS USA 82: 3320-3324) containing a synthetic PstI site (see construction of pSBS2520) to the coding sequence of the Arabidopsis oleosin. Standard molecular biology laboratory techniques (see eg: Sambrook et al. (1990) Molecular Cloning, 2^{nd} ed. Cold Spring Harbor Press) were again used to clone the HindIII KpnI fragment containing the phaseolin terminator (see construction of pSBS2520) dowstream of the Trxh gene. The PstI-phaseolin promoter-oleosin-Trxh-phaseolin terminator-KpnI insert sequence was cloned into the PstI-KpnI sites of pSBS3000. The resulting plasmid is called pSBS2510. The sequence of the phaseolin promoter-oleosin Trxh-phaseolin terminator sequence is shown in SEQ ID NO:16.

Construction of Plant Transformation Vector pSBS2521.

This vector contains the same genetic elements as the insert of pSBS2510 except the Trxh gene is fused to the 5′ end of the oleosin gene. The 3′ oleosin coding sequence including its native stopcodon (van Rooijen et al (1992) Plant Mol. Biol.18: 1177-1179) was furnished with a HindIII cloning site. Again a gene splicing by overlap extension technique (Horton et al (1989) 15: 61-68) was used to fuse the phaseolin promoter to the Trxh gene and to fuse the Trxh gene to the oleosin sequence. Standard molecular biology laboratory techniques (see eg: Sambrook et al. (1990) Molecular Cloning, 2^{nd} ed. Cold Spring Harbor Press) were again used to clone the HindIII KpnI fragment containing the phaseolin terminator (see construction of pSBS2520) dowstream of the oleosin gene. The PstI-phaseolin promoter-Trxh oleosin-phaseolin terminator-KpnI insert sequence was cloned into the PstI-KpnI sites of pSBS3000. The resulting plasmid is called pSBS2521. The sequence of the phaseolin promoter-Trxh oleosin-phaseolin terminator sequence is shown in SEQ ID NO:19.

Construction of Plant Transformation Vector pSBS2527.

The Arabidopsis NADPH thioredoxin-reductase gene as described in example 1 was placed under the regulatory control of the phaseolin promoter and the phaseolin terminator derived from the common bean Phaseolus vulgaris (Slightom et al (1983) Proc. Natl Acad Sc USA 80: 1897-1901; Sengupta-Gopalan et al., (1985) PNAS USA 82: 3320-3324). A gene splicing by overlap extension technique (Horton et al (1989) 15: 61-68) was used to fuse the phaseolin promoter to the thioredoxin-reductase gene. Standard molecular biology laboratory techniques (see eg: Sambrook et al. (1990) Molecular Cloning, 2^{nd} ed. Cold Spring Harbor Press) were used to furnish the phaseolin promoter and terminator with PstI and HindIII/KpnI sites respectively (see SEQ ID NO:14). Standard molecular biology laboratory techniques were also used to place the phaseolin terminator dowstream from the thioredoxin-reductase gene. The PstI-phaseolin promoter-thioredoxin-reductase-phaseolin terminator-KpnI insert sequence was cloned into the PstI-KpnI sites of pSBS3000 The resulting plasmid is called pSBS2527. The sequence of the phaseolin promoter-Arabidopsis thioredoxin-reductase-phaseolin terminator sequence is shown in SEQ ID NO:22.

Construction of Plant Transformation Vector pSBS2531.

A gene splicing by overlap extension technique (Horton et al (1989) 15: 61-68) was used to fuse the phaseolin promoter (Slightom et al (1983) Proc. Natl Acad Sc USA 80: 1897-1901; Sengupta-Gopalan et al., (1985) PNAS USA 82: 3320-3324) to the coding sequence of the Arabidopsis oleosin. The same gene splicing technique was used to fuse the oleosin gene to the thioredoxin-reductase coding sequence. Standard molecular biology laboratory techniques (see eg: Sambrook et al. (1990) Molecular Cloning, 2^{nd} ed. Cold Spring Harbor Press) were again used to clone the HindIII KpnI fragment containing the phaseolin dowstream of the thioredoxin-reductase gene. The PstI-phaseolin promoter-oleosin-thioredoxin-reductase-phaseolin terminator-KpnI insert sequence was cloned into the PstI-KpnI sites of pSBS3000. The resulting plasmid is called pSBS2531. The sequence of the phaseolin promoter-oleosin thioredoxin-reductase-phaseolin terminator sequence is shown in SEQ ID NO:24.

Construction of Plant Transformation Vector pSBS2529

This vector contains the same genetic elements as the insert of pSBS2531 except the thioredoxin-reductase gene is fused to the 5′ end of the oleosin gene. The 3′ oleosin coding sequence including its native stopcodon (van Rooijen et al. (1992) Plant Mol. Biol.18: 1177-1179) was furnished with a HindIII cloning site. Again a gene splicing by overlap extension technique (Horton et al (1989) 15: 61-68) was used to fuse the phaseolin promoter to the thioredoxin-reductase gene and to fuse the thioredoxin-reductase gene to the oleosin sequence. Standard molecular biology laboratory techniques (see eg: Sambrook et al. (1990) Molecular Cloning, 2^{nd} ed. Cold Spring Harbor Press) were again used to clone the HindIII KpnI fragment containing the phaseolin terminator (see construction of pSBS2520) dowstream of the oleosin gene. The PstI-phaseolin promoter-thioredoxin-reductase oleosin-phaseolin terminator-KpnI insert sequence was cloned into the PstI-KpnI sites of pSBS3000. The resulting plasmid is called pSBS2529. The sequence of the phaseolin promoter-thioredoxin-reductase oleosin-phaseolin terminator sequence is shown in SEQ ID NO:27.

Construction of Plant Transformation Vector pSBS2530.

A plant transformation was constructed containing the Mycobacterium Leprae thioredoxin-reductase/thioredoxin gene (Mlep TR/Trxh). A construct called pHIS/TR/Trxh (Wieles et al (1995) J Biol Chem 270:25604-25606) was obtained from the department of Immunohematology and Blood bank, Leiden University, The Netherlands and use as a template for PCR to generate pSBS2530. The construction of pSBS2530 was identical to the construction of pSBS2531 except that the Mlep TR/Trxh gene was used instead of the Arabidopsis thioredoxin-reductase gene. A gene splicing by overlap extension technique (Horton et al (1989) 15: 61-68) was used to fuse the phaseolin promoter (Slightom et al (1983) Proc. Natl Acad Sc USA 80: 1897-1901; Sengupta-Gopalan et al., (1985) PNAS USA 82: 3320-3324) to the coding sequence of the Arabidopsis oleosin. The same gene splicing technique was used to fuse the oleosin gene to the Mlep TR/Trxh coding sequence. Standard molecular biology laboratory techniques (see eg: Sambrook et al. (1990) Molecular Cloning, 2^{nd} ed. Cold Spring Harbor Press) were again used to clone the HindIII-KpnI fragment containing the phaseolin dowstream of the Mlep TR/Trxh gene. The PstI-phaseolin promoter-oleosin Mlep TR/Trxh-phaseolin terminator-KpnI insert sequence was cloned into the PstI-KpnI sites of pSBS3000. The resulting plasmid is called pSBS2530. The sequence of the phaseolin promoter-oleosin Mlep TR/Trxh-phaseolin terminator sequence is shown in SEQ ID NO:30.

Construction of Plant Transformation Vector pSBS2542.

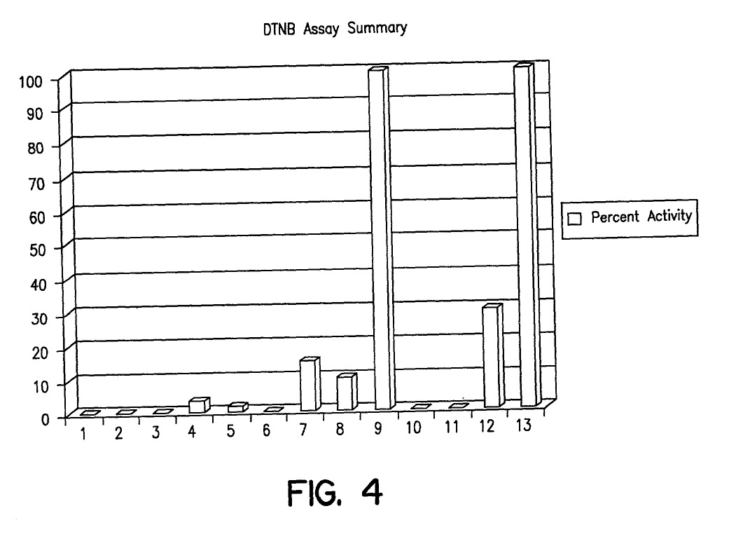

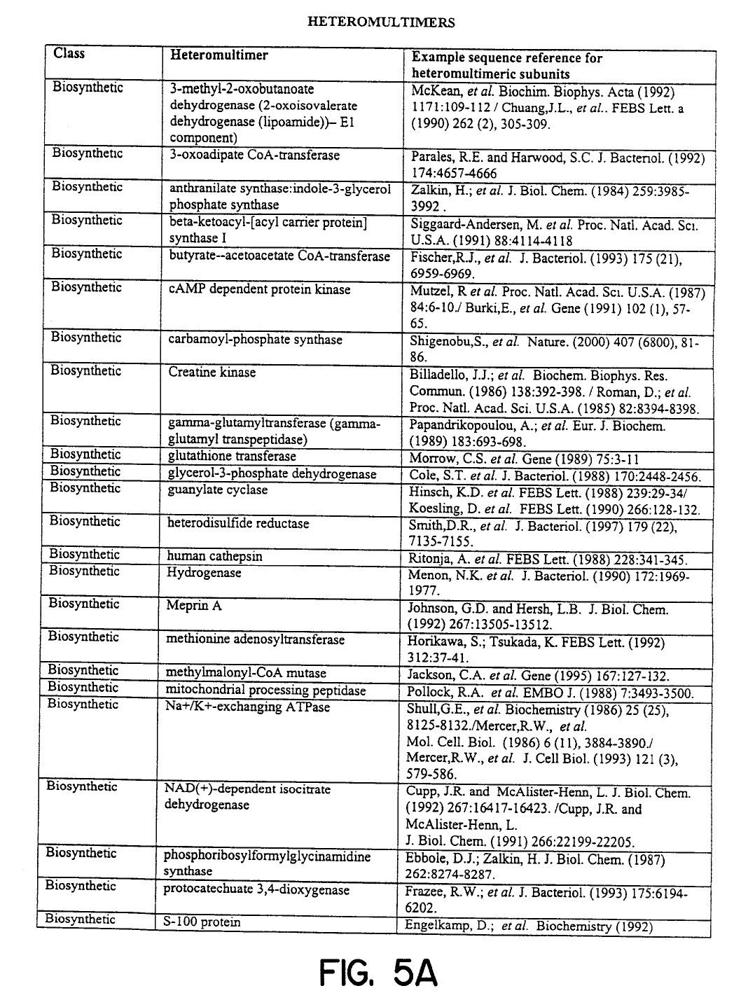

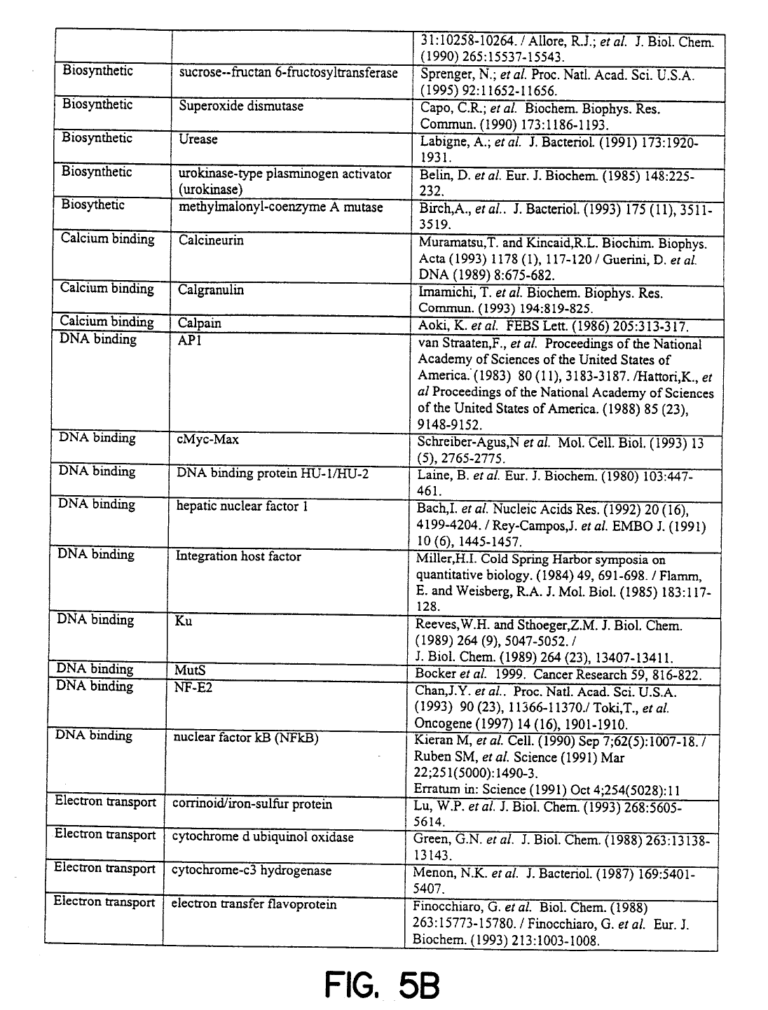

From initial activity assays (FIG. 4), it was apparent that oil bodies expressing the oleosin-M. lep TR/Trxh fusion protein contained considerable reducing activity. It was anticipated that a similar oleosin fusion construct encoding the Arabidopsis thioredoxin-reductase and thioredoxin proteins would behave in an analogous manner. Molecular modeling was used to aid in the design of such a construct. Primers were designed (thioredoxin link-L: 5′-ACTGGAGATGTTGACTCGACGGATACTACGGATTGGTCGACGG CTATGGAAGAAGGACAAGTGATCGCCTGC-3′; (SEQ ID NO:5), and thioredoxin link-R:

5′-ATCCGTCGAGTCAACATCTCCAGTTTCCTCGGTGGTCTCGTTAGCCT TCGATCCAGCAATCTCTTGTAAGAATGCTCTGC-3′; (SEQ ID NO:6) to code for a synthetic linker peptide between the thioredoxin-reductase and thioredoxin proteins. These primers were used in conjunction with primers GVR 873 (5′-GTGGAAGCT TATGGAGATGGAG-3′; SEQ ID NO:7) and GVR834 (5′-GAAAGCTTAAGCCAAGTGTTTG-3′; SEQ ID NO:2) to amplify a region coding for a thioredoxin-reductase-linker region-thioredoxin utilizing a gene splicing by overlap extension technique (Horton et al (1989) 15:61-68). The thioredoxin-reductase-linker-thioredoxin encoding sequence was then cloned into a pre-existing pSBS3000 vector using standard molecular biology techniques (Sambrook et al (1990) Molecular Cloning 2^{nd} Edition Cold Spring Harbour Press). The resulting plasmid was called pSBS2542. The sequence of the phaseolin promoter-oleosin-thioredoxin-reductase-linker-thioredoxin-phaseolin terminator region is shown in SEQ ID NO:33. An amino acid sequence comparison between this Arabidopsis thioredoxin-reductase-linker-thioredoxin and the M. leprae TR/Trxh protein is shown in FIG. 12.

Plasmids pSBS2510, pSBS2520, pSBS2521, pSBS2527, pSBS2529, pSBS2530, pSBS2531 and pSBS2542 were electroporated into Agrobacterium strain EHA101. These Agrobacterium strains were used to transform Arabidopsis. Arabidopsis transformation was done essentially as described in “Arabidopsis Protocols; Methods in molecular biology Vol 82. Edited by Martinez-Zapater J M and Salinas J. ISBN 0-89603-391-0 pg 259-266 (1998) except the putative transgenic plants were selected on agarose plates containing 80 μM L-phosphinothricine, after they were transplanted to soil and allowed to set seed.

### EXAMPLE 3

Polyacrylamide Gelelectrophoresis and Immunoblotting of Transgenic Seed Extracts.

Source of Arabidopsis Thioredoxin, Thioredoxin-Reductase and Oleosin Antibodies.

The Arabidopsis thioredoxin and thioredoxin-reductase genes were cloned in frame in bacterial expression vector pRSETB (Invtrogen) to allow for the overexpression of Arabidopsis thioredoxin and thioredoxin-reductase proteins. These proteins were purified using standard protocols (see eg Invitrogen protocol) and used to raise antibodies in rabbits using standard biochemical techniques (See eg Current Protocols in Molecular Biology, John Wiley & Sons, N.Y. (1989). The Arabidopsis oleosin gene genes was cloned in frame in bacterial expression vector pRSETB (Invitrogen) to allow for the overexpression Arabidopsis oleosin protein. This protein was purified using standard protocols (see eg Invitrogen protocol) and used to prepare mouse monoclonal antibodies using standard biochemical techniques (See eg Current Protocols in Molecular Biology, John Wiley & Sons, N.Y. (1989).

Preparation of Total Arabidopsis Seed Extracts for PAGE.

Arabidopsis seeds were ground in approximately 20 volumes of 2% SDS, 50 mM Tris-Cl, this extract was boiled, spun and the supernatant was prepared for polyacrylamide gelelectrophoresis (PAGE) using standard protocols.

Preparation of Arabidopsis Oil-Body-Protein Extracts.

Arabidopsis seeds were ground in approximately 20 volumes of water and spun in a microfuge. The oilbodies were recovered and washed sequentially with approximately 20 volumes of water, a high stringency wash buffer, containing 8M urea and 100 mM sodiumcarbonate and water. After this last wash the oilbodies are prepared for poly acrylamide gelelectrophoresis (PAGE) using standard protocols.

Analysis of Seed and Oil Body Extracts from Plants Transformed with pSBS2510

Total seed and oilbody protein extracts from plants transformed with pSBS2510 were loaded onto polyacrylamide gels and either stained with coomassie brilliant blue or electroblotted onto PVDF membranes. The membranes were challenged with with a polyclonal antibody raised against Arabidopsis thioredoxin, or a monoclonal antibody raised against the Arabidopsis 18.5 kDa oleosin and and visualized using alkaline phosphatase. Expression of the oleosin-thioredoxin results in an additional band of 31.2 kDa. The results indicate that the thioredoxin antibodies are immunologically reactive with a band of the right predicted molecular weight (31.2 kDa), and the oleosin antibodies are also immunologically reactive with a band of the right predicted molecular weight for the fusion protein (31.2 kDa) in addition to a band corresponding to the native Arabidopsis oleosin (18.5 kDa). This indicates that oleosin-thioredoxin is expressed in Arabidopsis seeds and is correctly targeted to oilbodies.

Analysis of Seed and Oil Body Extracts from Plants Transformed with pSBS2521

Total seed and oilbody protein extracts from plants transformed with pSBS25121 were loaded onto polyacrylamide gels and either stained with Coomassie brilliant blue or electroblotted onto PVDF membranes. The membranes were challenged with with a polyclonal antibody raised against Arabidopsis thioredoxin, or a monoclonal antibody raised against the Arabidopsis 18.5 kDa oleosin and and visualized using alkaline phosphatase. Expression of the thioredoxin-oleosin results in an additional band of 31.2 kDa. The results indicate that the thioredoxin antibodies are immunologically reactive with a band of the right predicted molecular weight (31.2 kDa), and the oleosin antibodies are also immunologically reactive with a band of the right predicted molecular weight for the fusion protein (31.2 kDa) in addition to a band corresponding to the native Arabidopsis oleosin (18.5 kDa). This indicates that thioredoxin-oleosin is expressed in Arabidopsis seeds and is correctly targeted to oilbodies.

Analysis of Seed Extracts from Plants Transformed with pSBS2520

Total seed extracts from plants transformed with pSBS2520 were loaded onto polyacrylamide gels and either stained with Coomassie brilliant blue or electroblotted onto PVDF membranes. The membranes were challenged with with a polyclonal antibody raised against Arabidopsis thioredoxin and visualized using alkaline phosphatase. The results indicated that the thioredoxin antibodies are immunologically reactive with a band of approximately the right predicted molecular weight (12 kDa). Untransformed seeds do not show a detectable thioredoxin band.

Analysis of Seed and Oil Body Extracts from Plants Transformed with pSBS2529

Total seed and oilbody protein extracts from plants transformed with pSBS2529 were loaded onto polyacrylamide gels and electroblotted onto PVDF membranes. The membranes were challenged with with a polyclonal antibody raised against Arabidopsis thioredoxin-reductase, or a monoclonal antibody raised against the Arabidopsis 18.5 kDa oleosin and and visualized using alkaline phosphatase. Expression of the thioredoxin-reductase-oleosin results in an additional band of 53.8 kDa. The results indicate that the thioredoxin-reductase antibodies are immunologically reactive with a band of the right predicted molecular weight for the fusion protein (53.8 kDa), the oleosin antibodies are also immunologically reactive with a band of the right predicted molecular weight (53.8 kDa) in addition to a band corresponding to the native Arabidopsis oleosin (18.5 kDa). This indicates that thioredoxin-reductase-oleosin is expressed in Arabidopsis seeds.

Analysis of Seed Extracts from Plants Transformed with pSBS2527

Total seed extracts from plants transformed with pSBS2527 were loaded onto polyacrylamide gels and electroblotted onto PVDF membranes. The membranes were challenged with with a polyclonal antibody raised against Arabidopsis thioredoxin-reductase and visualized using alkaline phosphatase. The thioredoxin-reductase antibodies are immunologically reactive with a band of approximately the right predicted molecular weight for the (35.3 kDa). Untransformed seeds do not show a detectable thioredoxin band.

Analysis of Seed Extracts from Plants Transformed with pSBS2531

A protein gel and immunoblot was prepared assaying the expression of oleosin-DMSR in Arabidopsis T2 seeds and correct targeting to Arabidopsis oilbodies. The expected molecular weight based on the deduced amino acid sequence is calculated to be 53,817 Da. In the oilbody extract of the transgenic oleosin-thioredoxin-reductase sample an extra band of approximately 54 kDa was observed. This band was confirmed to be oleosin-thioredoxin-reductase by immunoblotting. From the polyacrylamide gel it was observed that the expression of the oleosin-Thioredoxin-reductase is about double compared to the expression of the major 18.5 kDa Arabidopsis oleosin. This represents approximately 2-4% of total seed protein.

Analysis of Seed Extracts from Plants Transformed with pSBS2530

A protein gel and immunoblot was prepared assaying the expression of oleosin-M.lep TR/Trxh in Arabidopsis T2 seeds and the correct targeting to Arabidopsis oilbodies. The expected molecular weight based on the deduced amino acid sequence is calculated to be 67,550 Da. In the oilbody extract of the transgenic oleosin-M.lep TR/Trxh sample an extra band of approximately 68 kDa was observed. This band was confirmed to be oleosin-M.lep TR/Trxh by immunoblotting. From the polyacrylamide gel it was observed that the expression of the oleosin-M.lep TR/Trxh is similar to the expression of the major 18.5 kDa Arabidopsis oleosin. This represents approximately 1-2% of total seed protein.

Analysis of Seed Extracts from Plants Transformed with pSBS2542

Crude oil body extracts from pSBS2542 lines were prepared by grinding 100 μg of seed in 1 mL of 100 mM Tris buffer at pH 7.5. The samples were then centrifuged in order to isolate the oil body fraction. The oil body fraction was then loaded on an SDS polyacrylamide gel for expression analysis. A Coomassie stained gel revealed that the synthetic fusion accumulated to high levels in crude oil body extracts from 3 of the 4 lines tested. It was estimated that the fusion protein represented approximately 2-5% of total seed protein. Furthermore, western blots utilizing either anti-thioredoxin or anti-thioredoxin-reductase antibodies confirmed that the over expressed 70 kDa protein was indeed oleosin-thioredoxin-reductase-linker-thioredoxin.

### EXAMPLE 4

Biological Activity of Thioredoxin and Thioredoxin-Reductase Transformants

Initial Reduction Assays:

DTNB Assay

The activity of the thioredoxin and thioredioxin reductase was determined using a colorimetric DTNB [5,5′-dithiolbis (2-nitrobenzoic acid)] assay. The assay was performed in a 700 μL reaction volume containing 100 mM Tris-Cl pH 8.0, 5 mM EDTA, 200 μM DTNB [5,5′-dithiolbis (2-nitrobenzoic acid)] and 200 μM NADPH. If thioredoxin-reductase and thioredoxin are added, NADPH will reduce the thioredoxin-reductase, which will then reduce thioredoxin, which will, in turn, reduce DTNB (see equations below).

NADPH₂+thioredoxin-reductaseₒₓ→thioredoxin-reductase_{red}+NADP⁺

thioredoxin-reductase_{red}+thioredoxinₒₓ→thioredoxin_{red}+thioredoxin-reductaseₒₓ

thioredoxin_{red}+DTNBₒₓ→2(2-nitro-5-mercaptobenzoic acid)+thioredoxinₒₓ

The formation of the yellow product was monitored by measuring the OD₄₁₂ in a spectrophotometer after a set period of time (usually 0.5-2 hours). The results of initial activity assays are shown in the bar graph in FIG. 4 and described below.

Initially, 100 μg of total seed proteins were added from each of the Arabidopsis transgenic lines, pSBS2520 (cytosolic thioredoxin) and pSBS2527 (cytosolic thioredoxin-reductase), which corresponds to approximately 1 μg of cytosolic thioredoxin and thioredoxin-reductase used in the assay. In this case, the amount of DTNB reduced was comparable to the reduction caused by 1 μg each of E. coli thioredoxin and thioredoxin-reductase. In these plant seed samples, background readings were very low when only one of the 2 extracts (either cytosolic thioredoxin or cytosolic thioredoxin-reductase; FIG. 4, bars 3 and 6, respectively) was added to the reaction, along with wild type oil bodies.

Analysis with oil body fractions from transgenic seeds revealed that Arabidopsis thioredoxin and thioredoxin-reductase were substantially less active when fused to oleosins on oil bodies. Approximately 300 μg of crude, unwashed oil-body-protein was used in the assay (which corresponds to 10-30 μg of thioredoxin-oleosin (pSBS 2521; FIG. 4, bar 2), oleosin-thioredoxin (pSBS 2510, FIG. 4, bar 1), thioredoxin-reductase-oleosin (pSBS 2529, FIG. 4, bar 5), or oleosin-thioredoxin-reductase (PSBS 2531, FIG. 4, bar 4). The oil-body-proteins were tested in conjunction with 100 μg of total seed protein containing approximately 1 μg of cytosolic thioredoxin (pSBS 2520) or thioredoxin-reductase (pSBS 2527).

In such assays, pSBS2529 (thioredoxin-reductase-oleosin) and pSBS2531 (oleosin-thioredoxin-reductase) do contain reductase activity when combined with cytosolic thioredoxin from pSBS2520 (see FIG. 4, bars 7 and 8, respectively). Experiments estimated that the reductase activity of oleosin-thioredoxin-reductase was about 10-15% that of the cytosolic thioredoxin-reductase. The addition of tween at a final concentration of 0.4% could enhance this activity 2 or 3 fold. Interestingly, oleosin-thioredoxin-reductase (pSBS 2531) appears to be capable of reducing DTNB in the absence of added thioredoxin, although added thioredoxin causes significantly more DTNB reduction (see FIG. 4; compare bar 4 W.T.+oleosin-thioredoxin-reductase to bar 7 thioredoxin+oleosin-thioredoxin-reductase). Experiments with pSBS2521 (thioredoxin-oleosin) or pSBS2510 (oleosin-thioredoxin) combined with cytosolic thioredoxin-reductase from pSBS2527 (see FIG. 4, bars 10 and 11, respectively) indicate that thioredoxin activity of these fusions is undetectable at these concentrations.

Oil bodies from the transgenic Arabidopsis line, pSBS2530 (oleosin-M.lep TR/Trxh) contain significant thioredoxin/thioredoxin-reductase activity (see FIG. 4, bar 12). One hundred micrograms of crude oilbody protein for pSBS2530 was tested (corresponding to approximately 5 μg of oleosin-M.lep TR/trxh fusion) in the assay. Based on the assay, it was estimated that this fusion is about 25-40% as active as cytosolic Arabidopsis thioredoxin and thioredoxin-reductase (FIG. 4, bar 9) when comparing specific activity.

Insulin Reduction Assay

The results from the DTNB assays were confirmed with insulin reduction assays. This assay contained insulin at a final concentration of 1 mg/mL in 100 mM KH₂PO₄ pH 7.0+5 mM EDTA. In the presence of NADPH (500 μM), thioredoxin, and thioredoxin-reductase, insulin is reduced and precipitates from the solution. Normally, insulin reduction is followed by measuring turbidity at OD 650. Alternatively, one can measure the conversion of NADPH₂ to NADP⁺ by monitoring the decrease in absorbance at 340 nm.

Both of the assays are difficult to measure when oil bodies are present, due to interference with the spectrophotometer readings. However, qualitative data could be obtained by centrifuging the tubes after a set period of time, and determining if an insulin pellet was present (oil bodies float to the top, while the insulin precipitate pellets out). Alternatively, samples could be filtered after a set period of time, and the change in absorbance at 340 nm could be measured. As mentioned previously, the results of the insulin reduction assays agreed with those of the DTNB assay, with the exception of the observation that pSBS2531 (oleosin-thioredoxin-reductase) only reduced insulin in the presence of free thioredoxin from pSBS2520.

Assays on Seeds from Arabidopsis Crosses that Co-Express Oleosin-Thioredoxin and Oleosin-Thioredoxin-Reductase.

Based upon initial DTNB and insulin reduction assays, it was apparent that mixing oil bodies from oleosin <−> thioredoxin and oleosin <−> thioredoxin-reductase transgenic seeds resulted in very limited reducing activity (Note: the <−> indicates both configurations of oleosin fusions; ie. oleosin <−> thioredoxin would represent oleosin-thioredoxin and thioredoxin-oleosin fusions).

To determine whether having oleosin <−> thioredoxin and oleosin <−> thioredoxin-reductase proteins present on the same oil body would have a positive effect on the reducing activity of these proteins, crosses were set up to generate double transgenic Arabidopsis lines. The crosses are illustrated in Table 2.

TABLE 2

| | | | Confirmed |
|---|---|---|---|
| | | | double |
| | | | transgenic lines |
| | | | (PCR and |
| Male | | Female | Western Blot) |
| oleo-thioredoxin | X | oleo-thioredoxin- | 4 |
| | | reductase | |
| oleo-thioredoxin | X | thioredoxin-reductase- | 1 |
| | | oleo | |
| thioredoxin-oleo | X | oleo-thioredoxin- | 0 |
| | | reductase | |
| thioredoxin-oleo | X | thioredoxin-reductase- | 4 |
| | | oleo | |
| oleo-thioredoxin- | X | oleo-thioredoxin | 2 |
| reductase | | | |
| oleo-thioredoxin- | X | thioredoxin-oleo | 0 |
| reductase | | | |
| thioredoxin- | X | oleo-thioredoxin | 7 |
| reductase-oleo | | | |
| thioredoxin- | X | thioredoxin-oleo | 0 |
| reductase-oleo | | | |

Seeds from Arabidopsis crosses were germinated on PPT plates and the seedlings were transferred to soil after approximately 2 weeks. PCR experiments on DNA isolated from the seedlings identified a number of plants which contain both an oleosin <−> thioredoxin and an oleosin <−> thioredoxin-reductase gene construct within their genome.

Seeds were harvested from these plants for expression and activity assays. Western blots were carried out to confirm expression of both oleosin <−> thioredoxin and oleosin <−> thioredoxin-reductase in the lines. DTNB and insulin reduction assays were also performed to compare activity between single transgenic parent lines and the double transgenic offspring and results are summarized in Table 3. Table 3 summarizes DTNB reducing activity of various transgenic lines. The last 2 rows compare mixing oil bodies from single transgenic parent lines to using oil bodies from double transgenic offspring. Relative activity for the E. coli thioredoxin and thioredoxin mixture is set at 100 percent.

TABLE 3

| | Relative Activity |
|---|---|
| Source Material | (%) |
| E. coli trx + NTR | 100 |
| Arabidopsis “free” thioredoxin + | 100 |
| thioredoxin-reductase | |
| (pSBS2520 + pSBS2527) | |
| oleosin- M. lep TR/Trxh | ˜30 |
| (pSBS2530) | |
| Oleosin <-> thioredoxin-reductase + | ˜3 |
| oleosin <-> thioredoxin (mixing oil | |
| bodies from single-transgenic parents) | |
| Oleosin <-> thioredoxin-reductase × | ˜50 |
| oleosin <-> thioredoxin | |
| (various double transgenic lines) | |

Based on DTNB and insulin reduction assays, it is evident that double transgenic plants co-expressing oleosin <−> thioredoxin and oleosin <−> thioredoxin-reductase on the same, single oil body contained significantly more reducing activity compared to mixing oil bodies from single transgenic oleosin <−> thioredoxin and oleosin <−> thioredoxin-reductase lines. It was additionally apparent that oil body extracts from co-expressing lines contained more reducing activity compared to line pSBS2530 (oleosin-M. lep TR/Trxh), which was previously identified as the line containing the highest reducing activity from oil bodies.

These results suggest that the creation of double transgenic lines (either through crossing or by co-transforming 2 expression constructs into plants) may represent one means by which we could solve our initial problem of not being able to generate reducing activity by mixing oil bodies from oleosin <−> thioredoxin and oleosin <−> thioredoxin-reductase single transgenic lines.

Assays on Seeds from Arabidopsis pSBS2542 Transgenic Lines that Express Oleosin-Thioredoxin-Reductase-Linker-Thioredoxin.

Oil body extracts from four pSBS2542 lines were tested for reducing activity in DTNB and insulin reduction assays, using standard protocols described previously. Again, oil body extracts containing the oleosin-thioredoxin-reductase-linker-thioredoxin protein possessed significant reducing activity. Based on such assays, it was revealed that the oleosin-thioredoxin-reductase-linker-thioredoxin synthetic fusion protein was more active than the oleosin-M. lep TR/Trxh fusion. Furthermore, oil bodies containing the oleosin-thioredoxin-reductase-linker-thioredoxin protein appeared to have more reducing activity compared to oil bodies from double transgenic lines that co-expressed oleosin <−> thioredoxin and oleosin <−> thioredoxin-reductase. The results comparing reducing activity for the various thioredoxin-reductase/thioredoxin constructs is summarized in Table 4. Table 4 summarizes DTNB reducing activity of various transgenic lines. The pSBS2542 line expressing oleosin-thioredoxin-reductase-linker-thioredoxin contains significant reducing activity, comparable to the “free” forms of Arabidopsis thioredoxin and thioredoxin-reductase and the equivalent E. coli proteins. Relative activity for the E. coli thioredoxin and thioredoxin mixture is set at 100 percent.

TABLE 4

| | Relative Activity |
|---|---|
| Source Material | (%) |
| E. coli trx + NTR | 100 |
| Arabidopsis “free” thioredoxin + thioredoxin- | 100 |
| reductase | |
| (pSBS2520 + pSBS2527) | |
| oleosin- M. lep TR/Trxh | ˜30 |
| (pSBS2530) | |
| Oleosin <-> thioredoxin-reductase + | ˜3 |
| oleosin <-> thioredoxin | |
| (mixing oil bodies from single-transgenic | |
| parents) | |
| Oleosin <-> thioredoxin-reductase × | ˜50 |
| oleosin <-> thioredoxin | |
| (various double transgenic lines) | |
| Oleosin-thioredoxin- | ˜75-100 |
| reductase-linker-thioredoxin | |
| (pSBS2542) | |

Reduction Assays Comparing the Utilization of NADH vs. NADPH as a Cofactor (Electron Donor) for the Thioredoxin-Reductase/Thioredoxin System.

DTNB and insulin reduction assays were conducted as described previously, except that NADH was substituted for NADPH as an electron donor in the system utilizing E. coli thioredoxin-reductase and thioredoxin. Thus, a comparison was conducted of the utilization of NADH versus NADPH as a cofactor for the E. coli thioredoxin-reductase/thioredoxin system. For the DTNB assay, the reaction mixture consisted of 400 μM DTNB, 10 μg/mL E. coli thioredoxin, and 10 μg/mL E. coli thioredoxin-reductase in 100 mM Tris-Cl buffer pH 8.0. Either NADH or NADPH was then added to the DTNB reaction as follows:

Reaction A. 200 μM NADPH (Sigma)

Reaction B. 800 μM NADH (Sigma)

Reaction C. 800 μM NADH (Roche)

Reaction D. (−) cofactor

Reaction E. 800 μM NADH (no TR or Trxh).

For the insulin reduction assay, the reaction mixture consisted of 1 mg/mL bovine pancreatic insulin, 20 μg/mL E. coli thioredoxin, and 20 μg/mL E. coli thioredoxin-reductase in 100 mM potassium phosphate buffer at pH 7.0. Either NADH or NADPH was then added to the reaction as follows:

Reaction A. 800 μM NADPH (Sigma)

Reaction B. 800 μM NADH (Sigma)

Reaction C. 800 μM NADH (Roche)

Reaction D. (−) cofactor

Reaction E. 2 mM NADH (no TR or Trxh).

The results indicate that NADH, purchased from either Sigma or Roche, could act as an electron donor in both the DTNB and insulin reduction assays. However, the rate of reduction was lower than the rate observed with NADPH as a cofactor. It was estimated that the rate of insulin reduction utilizing NADH as an electron donor was approximately 25-50% when compared to the maximum rate using NADPH. Furthermore, it was estimated that the rate of DTNB reduction utilizing NADH as an electron donor was approximately 5-10% of the maximum rate using NADPH. Similar results were observed using the oleosin-thioredoxin-reductase-linker thioredoxin fusion protein on Arabidopsis oil bodies instead of the E. coli thioredoxin-reductase and thioredoxin.

### EXAMPLE 5

Production of Multimeric Immunoglobulin Protein in Plant Seed Cells and Capture on Oil Bodies Using Protein A—Oleosin Fusion Proteins.

1—Production of Multimeric Immunoglobulin Protein in Plant Seed Cells

For expression of multimeric-protein-complexes containing multimeric-immunoglobulin-complexes, the cDNA sequences encoding individual light and heavy chains can be isolated from; 1) cell lines expressing a particular antibody, such as clonal B cell lines, or a hybridoma cell line, or 2) may be a recombinant antibody, assembled by combining select light and heavy chain variable domains and available light and heavy chain constant domain sequences, respectively. Variable domains with specific binding properties may be isolated from screening populations of such sequences, usually in the form of a single-chain Fv phage display library.

Starting from known nucleic acid sequences and a source of light and heavy chains, the mature polypeptide coding sequences of each chain is isolated with a secretion signal sequence. The signal sequence can be the native antibody sequence or derived from a known secreted plant sequence (e.g. a PR sequence from Arabidopsis or tobacco). The addition of a plant secretion signal sequence to both light and heavy chain mature coding sequences is carried out by standard molecular biology techniques. PCR fusion is used routinely to make such modifications. Secretion signal sequences are included to target the light and heavy immunoglobulin polypeptides for secretion from the cell and further assembly of the two chains into a multimeric-immunoglobulin-complex. For expression in transgenic plant seeds, an expression cassette is assembled comprising: 1) a regulatory promoter sequence to provide expression in plant seeds, 2) the secretion signal—light chain sequence, and 3) a regulatory sequence to terminate transcription. A second expression cassette is assembled comprising: 1) a regulatory promoter sequence to provide expression in plant seeds, 2) the secretion signal—heavy chain sequence, and 3) a regulatory sequence to terminate transcription. Each of the antibody chain expression cassettes is cloned individually into an Agrobacterium plant transformation vector or is combined into a single transformation vector with both expression cassettes. In both cases, the expression cassettes are cloned into plant transformation vectors, between the left and right delineating border sequences, and adjacent to a plant selectable marker cassette. Each plant transformation vector is transformed into Agrobacterium. The resulting Agrobacterium strains are used to infect plant tissues. Transgenic plant material is regenerated and viable transgenic plants are selected. When individual transformation vectors are used, the transgenic plant lines that are produced, expressing either light or heavy chain sequences, are crossed to generate a single plant line expressing both chains in the same plant cell. When a single transformation vector, containing both light and heavy expression cassettes, is used, the initial transgenic plant line produces both light and heavy chain sequences in the same plant cell.

2—Production of Transgenic Oil Bodies which Display Protein A for the Capture of Immunoglobulins

To capture and display immunoglobulin protein on oil bodies, oil bodies are engineered to display an immunoglobulin binding protein. In this example, the well-known antibody-binding domains from Protein A are used. Based on the known sequence for Protein A from Staphylococcus aureus, PCR primers are designed to isolate the five consecutive Ig-binding domains from the bacterial Protein A sequence. Primers are designed to allow cloning of the Protein A sequence as either an N-terminal or C-terminal fusion to an oleosin sequence for targeting to oil bodies. The sequence that encodes an in-frame translational fusion between Protein A and oleosin is cloned into a plant expression cassette for seed-specific expression. The final cassette consists of a regulatory promoter sequence that provides expression in seeds, the Protein A—oleosin fusion sequence, and a regulatory sequence to terminate transcription. The Protein A—oleosin expression cassette is cloned into a plant transformation vector compatible with Agrobacterium—mediated plant transformation. The transformation vector comprises left and right border sequences flanking the Protein A—oleosin expression cassette and an adjacent plant selectable marker cassette. The Agrobacterium strain containing this vector is used to infect plant tissues and subsequent regeneration and selection from transgenic plant material to create transgenic plants.

3—Capture and Display of Multimeric-Immunoglobulins on Oil Bodies Displaying Protein A

Having produced light and heavy chain multimeric immunoglobulin complexes in one transgenic plant line and the display of Protein A on oil bodies through the oil body targeting of a Protein A—oleosin fusion protein in a second plant line, at least two embodiments can be used to capture the immunoglobulin complex on the Protein A oil bodies. In the first embodiment, transgenic seed from both the immunoglobulin and the Protein A—oleosin expression lines is combined in an optimum ratio and then ground together such that the disrupted material from both seed lines would be combined in the same extract. The combined seed extracts are mixed and/or incubated under conditions that allow maximum recovery of the immunoglobulin by Protein A. The oil body fraction is separated using standard phase separation techniques (e.g. centrifugation). The recovered oil body fraction contains both native oil bodies, from the immunoglobulin expression line, and transgenic Protein A oil bodies from the Protein A—oleosin expression line.

In a second embodiment, the plant lines expressing the immunoglobulin complex and the Protein A—oleosin fusion are crossed and individual plant lines expressing both components are identified and propagated. In this approach, the immunoglobulin complex and the Protein A—oleosin fusion are produced in different cellular compartments of the same plant seed cell. Seed from the double transgenic line is ground to disrupt the cellular material and mix the contents of all cellular compartments, including combining the immunoglobulin in the extracellular compartment and the Protein A—oleosin on the oil body in the cytosolic compartment. The material is mixed and/or incubated under conditions to allow maximum recovery of the immunoglobulin by Protein A, and the oil body fraction is separated by phase separation techniques. The recovered oil body fraction contains the displayed Protein A and the capture immunoglobulin complex.

### EXAMPLE 6

Production of Assembled Multimeric-Immunoglobulin-Complexess as Fusions with Oil Body Targeting Domains.

Individual polypeptides are produced as a fusion protein with oil body targeting sequences (e.g. oleosin) for display on oil bodies. It has been found that the individual subunits of naturally associating heterodimeric proteins can be co-produced as individual oleosin fusions and still associate as an active heterodimer on the surface of the oil body. In this example, the heterodimer is the light and heavy chain subunits, or derived portions thereof, of an immunoglobulin complex.

Production of an Immunoglobulin Fab Complex on Oil Bodies.

The mature light chain sequence, lacking the secretion signal sequence, is attached as an in-frame N-terminal fusion to an oleosin sequence. This fusion sequence is assembled into a seed-specific expression cassette consisting of a seed-specific promoter sequence, the light chain—oleosin fusion sequence, and a transcriptional terminator sequence. The expression cassette is inserted between the left and right border markers, adjacent to a plant selectable marker cassette, of a transformation vector. The transformation vector, in Agrobacterium, is used to infect plants and generate transgenic plants.

An equivalent construct for the heavy chain subunit, comprising the variable and constant heavy chain domains, is also attached as an in-frame fusion to oleosin and assembled into an expression cassette for seed-specific expression. The expression cassette can be a part of a separate transformation vector for the generation of a separate transgenic line, or the heavy chain expression cassette can be combined together with the light chain cassette into a single transformation vector. If light and heavy chain expression cassettes are transformed into plants on separate transformation vectors, the individual plant lines are crossed to create a single line expressing both heterodimer subunit—oleosin fusions in the same plant cell. Seed from the double transgenic line, or a single transgenic line generated from the dual expression vector, is extracted to isolate oil bodies. The seed material is ground to release the cellular contents and oil bodies are isolated by phase separation. The targeting of both light and heavy chain sequence to oil bodies, as oleosin fusions, allows the association of the immunoglobulin complex on the surface of the oil body.

Similar configurations, using the entire heavy chain sequence in combination with the entire light chain sequence, or using the variable domains from both the light and heavy chain sequences, are constructed to assemble different types of heteromultimeric-immunoglobulin-complexes (e.g., heterodimers) on the surface of oil bodies.

The present invention should therefore not be seen as limited to the particular embodiments described herein, but rather, it should be understood that the present invention has wide applicability with respect to protein expression generally. Since modifications will be apparent to those of skill in this art, it is intended that this invention be limited only by the scope of the appended claims.

### SUMMARY OF SEQUENCES

SEQ ID NOs:1-4 set forth primers which were synthesized for the isolation of the thioredoxin h (Trxh) and thioredoxin reductase genes from Arabidopsis, as described in Example 1.

SEQ ID NOs:5-7 set forth primers which were designed to code for a specific linker peptide between thioredoxin reductase and thioredoxin proteins, as described in Exmaple 2.

SEQ ID NOs:8, 10 and 11 set forth the nucleotide sequence and the deduced amino acid sequence of the NADPH thioredoxin reductase sequence isolated herein as described in Example 1.

SEQ ID NOs:9 and 11, respectivley, set forth the nucleotide sequence of the published NADPH thioredoxin reductase sequence (ATTHIREDB) and the deduced amino acid sequence.

SEQ ID NO:12 sets forth the deduced amino acid sequence of the published NADPH thioredoxin reductase sequence.

SEQ ID NO:13 sets forth the deduced amino acid sequence of the NADPH reductase sequence isolated in this report.

SEQ ID NOs:14 and 15 set forth the nucleotide sequence of the phaseolin promoter-Arabidopsis Trxh-phaseolin terminator sequence described in Example 2, and the deduced amino acid sequence. The Trxh coding sequence and its deduced amino acid sequence is indicated. The phaseolin promoter corresponds to nucleotide 6-1554, and the phaseolin terminator corresponds to nucleotide sequence 1905-3124. The promoter was furnished with a PstI site (nt 1-6) and the terminator was furnished with a HindIII site (nt 1898-1903) and a KpnI site (nt 3124-3129) to facilitate cloning.

SEQ ID NOs:16, 17 and 18 set forth the nucleotide sequence of the phaseolin promoter-oleosin Trxh-phaseolin terminator sequence described in Example 2, and the deduced amino acid sequences. The oleosin-Trxh coding sequence and the deduced amino acid sequences are indicated in SEQ ID NO:16. As in SEQ ID NO:14, the phaseolin promoter corresponds to nucleotide 6-1554. The sequence encoding oleosin corresponds to nt 1555-2313, the intron in this sequence (nt 1908-2147) is indicated in italics. The Trxh coding sequence corresponds to nt 2314-2658. The phaseolin terminator corresponds to nucleotide sequence 2664-3884.

SEQ ID NO:19, 20 and 21 set forth the nucleotide sequence of the phaseolin promoter-Trxh oleosin-phaseolin terminator sequence as described in Example 2, and the deduced amino acid sequences. The Trxh oleosin-coding sequence and its deduced amino acid sequences are indicated in SEQ ID NO:19. As in SEQ ID NOs:14 and 16, the phaseolin promoter corresponds to nucleotide 6-1554. The Trxh coding sequence corresponds to nt 1555-1896. The sequence encoding oleosin corresponds to nt 1897-2658, the intron in this sequence (nt 2250-2489) is indicated in italics. The phaseolin terminator corresponds to nucleotide sequence 2664-3884.

SEQ ID NO:22 and 23 set forth the nucleotide sequence of the phaseolin promoter-thioredoxin-reductase-phaseolin terminator sequence as described in Example 2, and the deduced amino acid sequence. The thioredoxin-reductase coding sequence and its deduced amino acid sequence is indicated in SEQ ID NO:22. The phaseolin promoter corresponds to nucleotide 6-1554. The thioredoxin-reductase coding sequence corresponds to nt 1555-2556 and the deduced amino acid is set forth in SEQ ID NO:23. The phaseolin terminator corresponds to nucleotide sequence 2563-3782.

SEQ ID NOs:24, 25 and 26 show the nucleotide sequence of the phaseolin promoter-oleosin thioredoxin-reductase-phaseolin terminator sequence as described in Example 2, and the deduced amino acid sequences. The oleosin-thioredoxin-reductase coding sequence and its deduced amino acid sequence is indicated. The phaseolin promoter corresponds to nucleotide 6-1554. The sequence encoding oleosin corresponds to nt 1555-2313, the intron in this sequence (nt 1980-2147) is indicated in italics. The thioredoxin-reductase coding sequence corresponds to nt 2314-3315. The phaseolin terminator corresponds to nucleotide sequence 3321-4540.

SEQ ID NOs:27, 28 and 29 show the nucleotide sequence of the phaseolin promoter-thioredoxin-reductase oleosin-phaseolin terminator sequence as described in Example 2, and the deduced amino acid sequences. The thioredoxin-reductase coding sequence and its deduced amino acid sequence is indicated. The phaseolin promoter corresponds to nucleotide 6-1554. The thioredoxin-reductase coding sequence corresponds to nt 1555-2553. The sequence encoding oleosin corresponds to nt 2554-3315, the intron in this sequence (nt 2751-3146) is indicated in italics. The phaseolin terminator corresponds to nucleotide sequence 3321-4540.

SEQ ID NO:30, 31 and 32 show the sequence of the phaseolin promoter-oleosin-Mlep thioredoxin-reductase/thioredoxin-phaseolin terminator sequence as described in Example 2, and the deduced amino acid sequences. The oleosin-Mlep thioredoxin-reductase/thioredoxin coding sequence and its deduced amino acid sequence is indicated. The phaseolin promoter corresponds to nucleotide 6-1554. The sequence encoding oleosin corresponds to nt 1555-2313, the intron in this sequence (nt) is indicated in italics. The Mlep thioredoxin-reductase/thioredoxin coding sequence corresponds to nt 2314-3690. The phaseolin terminator corresponds to nucleotide sequence 3698-4917.

SEQ ID NOs:33, 34 and 35 set forth the nucleotide sequence of the phaseolin promoter-oleosin-thioredoxin-reductase-linker-thioredoxin-phaseolin terminator region of pSBS2542, and the deduced amino acid sequences. The deduced amino acid sequence of oleosin-thioredoxin-reductase-linker-thioredoxin is also shown in SEQ ID NO:33. Amino acids representing oleosin are set forth at positions 1-173, those amino acids representing thioredoxin-reductase are set forth at positions 174-501, those amino acids representing the linker or spacer peptide are set forth at positions 501-524, and those representing thioredoxin are set forth at positions 525-636.

SEQ ID NOs:38 and 39 set forth the nucleotide sequence of Arabidopsis Thaliana Thioredoxin h (Trx h 1) and the encoded protein, respectively.

SEQ ID NOs:40 and 41 set forth the nucleotide sequence of Arabidopsis Thaliana Thioredoxin Reductase (NTR1) and the encoded protein, respectively.

SEQ ID NOs:42 and 43 set forth the nucleotide sequence of E. Coli Thioredoxin (TrxA) and the encoded protein, respectively.

SEQ ID NOs:44 and 45, set forth the nucleotide sequence of E. Coli Thioredoxin Reductase and the encoded protein, respectively.

SEQ ID NOs:46 and 47 set forth the nucleotide sequence of Human Thioredoxin and the encoded protein, respectively.

SEQ ID NOs:48 and 49, set forth the nucleotide sequence of Human Thioredoxin Reducatase and the encoded protein, respectively.

SEQ ID NOs:50 and 51, respsectively, set forth the nucleotide sequence of Mycobacterium leprae Thioredoxin-Thioredoxin Reducatase and the encoded protein, respectively.

SEQ ID NOs:52-313 are described in Table 5.

TABLE 5
EXAMPLES OF REDOX PROTEINS

| | |
|---|---|
| SEQ. ID | SWISS PROTEIN IDENTIFIED |
| NO. | (in parenthesis) |

PLANT THIOREDOXINS

Thioredoxin f-type

| | |
|---|---|
| 52 | (Q9XFH8) Thioredoxin F-type 1, chloroplast precursor |
| | (TRX-F1). - Arabidopsis thaliana (Mouse-ear cress) |
| 53 | (Q9XFH9) Thioredoxin F-type 2, chloroplast precursor |
| | (TRX-F2). {GENE: AT5G16400 OR MQK4.13} - |
| | Arabidopsis thaliana (Mouse-ear cress) |
| 54 | (O48897) Thioredoxin F-type, chloroplast precursor (TRX- |
| | F). {GENE: TRXF} - Brassica napus (Rape) |
| 55 | (O81332) Thioredoxin F-type, chloroplast precursor (TRX- |
| | F). - Mesembryanthemum crystallinum (Common ice |
| | plant) |
| 56 | (P29450) Thioredoxin F-type, chloroplast precursor (TRX- |
| | F). - Pisum sativum (Garden pea) |
| 57 | (P09856) Thioredoxin F-type, chloroplast precursor (TRX- |
| | F). - Spinicia oleracea (Spinach) |

Thioredoxin m-type

| | |
|---|---|
| 58 | (P06544) Thioredoxin 1 (TRX-1) (Thioredoxin M). {GENE: |
| | TRXA} - Anabaena sp. (strain PCC 7119) |
| 59 | (O48737) Thioredoxin M-type 1, chloroplast precursor |
| | (TRX-M1). {GENE: AT1G03680 OR F21B7_7 OR |
| | F21B7.28} - Arabidopsis thaliana (Mouse-ear cress) |
| 60 | (Q9SEU8) Thioredoxin M-type 2, chloroplast precursor |
| | (TRX-M2). {GENE: AT4G03520 OR F9H3.15 OR |
| | T5L23.1} - Arabidopsis thaliana (Mouse-ear cress) |
| 61 | (Q9SEU7) Thioredoxin M-type 3, chloroplast precursor |
| | (TRX-M3). {GENE: AT2G15570 OR F9O13.12} - |
| | Arabidopsis thaliana (Mouse-ear cress) |
| 62 | (Q9SEU6) Thioredoxin M-type 4, chloroplast precursor |
| | (TRX-M4). - Arabidopsis thaliana (Mouse-ear cress) |
| 63 | (Q9XGS0) Thioredoxin M-type, chloroplast precursor |
| | (TRX-M). - Brassica napus (Rape) |
| 64 | (P23400) Thioredoxin M-type, chloroplast precursor |
| | (TRX-M) (Thioredoxin CH2). {GENE: TRXM} - |
| | Chlamydomonas reinhardtii |
| 65 | (Q41864) Thioredoxin M-type, chloroplast precursor |
| | (TRX-M). {GENE: TRM1} - Zea mays (Maize) |
| 66 | (Q9ZP20) Thioredoxin M-type, chloroplast precursor |
| | (TRX-M). - Oryza sativa (Rice) |
| 67 | (P48384) Thioredoxin M-type, chloroplast precursor |
| | (TRX-M). - Pisum sativum (Garden pea) |
| 68 | (P07591) Thioredoxin M-type, chloroplast precursor |
| | (TRX-M). - Spinicia oleracea (Spinach) |
| 69 | (Q9ZP21) Thioredoxin M-type, chloroplast precursor |
| | (TRX-M). - Triticum aestivum (Wheat) |
| 70 | (P12243) Thioredoxin 1 (TRX-1) (Thioredoxin M). {GENE: |
| | TRXA OR TRXM} - Synechococcus sp. (strain PCC 7942) |
| | (Anacystis nidulans R2) |
| 71 | (P37395) Thioredoxin. {GENE: TRXA OR TRX} - |
| | Cyanidium caldarium [Chloroplast] |
| 72 | (O22022) Thioredoxin. {GENE: TRXA OR TRXM} - |
| | Cyanidioschyzon merolae [Chloroplast] |
| 73 | (P50338) Thioredoxin. {GENE: TRXA} - Griffithsia |
| | pacifica [Chloroplast] |
| 74 | (P50254) Thioredoxin. {GENE: TRXA} - Porphyra |
| | yezoensis [Chloroplast] |
| 75 | (P51225) Thioredoxin. {GENE: TRXA} - Porphyra |
| | purpurea [Chloroplast] |

Thioredoxin h-type

| | |
|---|---|
| 76 | (P29448) Thioredoxin H-type 1 (TRX-H-1). {GENE: TRX1 |
| | OR AT3G51030 OR F24M12.70} - Arabidopsis thaliana |
| | (Mouse-ear cress) |
| 77 | (P20857) Thioredoxin 2 (TRX-2). {GENE: TRXB} - |
| | Anabaena sp. (strain PCC 7120) |
| 78 | (Q42388) Thioredoxin H-type 1 (TRX-H-1) (Pollen coat |
| | protein). {GENE: THL-1 OR BOPC17} - Brassica napus |
| | (Rape), Brassica oleracea (Cauliflower) |
| 79 | (P29449) Thioredoxin H-type 1 (TRX-H1). - Nicotiana |
| | tabacum (Common tobacco) |
| 80 | (Q38879) Thioredoxin H-type 2 (TRX-H-2). {GENE: TRX2 |
| | OR AT5G39950 OR MYH19.14} - Arabidopsis thaliana |
| | (Mouse-ear cress) |
| 81 | (Q39362) Thioredoxin H-type 2 (TRX-H-2). {GENE: THL |
| | 2}- Brassica napus (Rape) |
| 82 | (Q07090) Thioredoxin H-type 2 (TRX-H2). - Nicotiana |
| | tabacum (Common tobacco) |
| 83 | (Q42403) Thioredoxin H-type 3 (TRX-H-3). {GENE: TRX3 |
| | OR AT5G42980 OR MBD2.18} - Arabidopsis thaliana |
| | (Mouse-ear cress) |
| 84 | (Q39239) Thioredoxin H-type 4 (TRX-H-4). {GENE: |
| | TRX4} - Arabidopsis thaliana (Mouse-ear cress) |
| 85 | (Q39241) Thioredoxin H-type 5 (TRX-H-5). {GENE: |
| | TRX5} - Arabidopsis thaliana (Mouse-ear cress) |
| 86 | (O64432) Thioredoxin H-type (TRX-H). {GENE: PEC-2} - |
| | Brassica rapa (Turnip) |
| 87 | (P80028) Thioredoxin H-type (TRX-H) (Thioredoxin CH1). |
| | {GENE: TRXH} - Chlamydomonas reinhardtii |
| 88 | (Q96419) Thioredoxin H-type (TRX-H). - Fagopyrum |
| | esculentum (Common buckwheat) |
| 89 | (Q42443) Thioredoxin H-type (TRX-H) (Phloem sap 13 |
| | kDa protein-1). - Oryza sativa (Rice) |
| 90 | (O65049) Thioredoxin H-type (TRX-H). {GENE: SBO9} - |
| | Picea mariana (Black spruce) |
| 91 | (Q43636) Thioredoxin H-type (TRX-H). - Ricinus |
| | communis (Castor bean) |
| 92 | (O64394) Thioredoxin H-type (TRX-H) (TrxTa). - Triticum |
| | aestivum (Wheat) |
| 93 | (P29429) Thioredoxin. - Emericella nidulans (Aspergillus |
| | nidulans) |

VIRUSES, BACTERIA AND FUNGI THIOREDOXINS

| | |
|---|---|
| 94 | (P80579) Thioredoxin (TRX). {GENE: TRXA} - |
| | Alicyclobacillus acidocaldarius (Bacillus acidocaldarius) |
| 95 | (O28137) Thioredoxin. {GENE: AF2145} - Archaeoglobus |
| | fulgidus |
| 96 | (P14949) Thioredoxin (TRX). {GENE: TRXA OR TRX} - |
| | Bacillus subtilis |
| 97 | (P00276) Thioredoxin. {GENE: NRDC} - Bacteriophage T4 |
| 98 | (O51088) Thioredoxin (TRX). {GENE: TRXA OR BB0061} |
| | - Borrelia burgdorferi (Lyme disease spirochete) |
| 99 | (P57653) Thioredoxin (TRX). {GENE: TRXA OR BU597} - |
| | Buchnera aphidicola (subsp. Acyrthosiphon pisum) |
| | (Acyrthosiphon pisum symbiotic bacterium) |
| 100 | (O51890) Thioredoxin (TRX). {GENE: TRXA} - Buchnera |
| | aphidicola (subsp. Schizaphis graminum) |
| 101 | (P10472) Thioredoxin (TRX). {GENE: TRXA} - Chlorobium |
| | limicola f.sp. thiosulfatophilum |
| 102 | (Q9PJK3) Thioredoxin (TRX). {GENE: TRXA OR TC0826} |
| | - Chlamydia muridarum |
| 103 | (Q9Z7P5) Thioredoxin (TRX). {GENE: TRXA OR CPN0659 |
| | OR CP0088} - Chlamydia pneumoniae (Chlamydophila |
| | pneumoniae) |
| 104 | (P52227) Thioredoxin (TRX). {GENE: TRXA} - Chlamydia |
| | psittaci (Chlamydophila psittaci) |
| 105 | (084544) Thioredoxin (TRX). {GENE: TRXA OR CT539} - |
| | Chlamydia trachomatis |
| 106 | (P00275) Thioredoxin C-1. - Corynebacterium nephridii |
| 107 | (P07887) Thioredoxin C-2. - Corynebacterium nephridii |
| 108 | (P52228) Thioredoxin C-3. - Corynebacterium nephridii |
| 109 | (P09857) Thioredoxin (TRX). {GENE: TRXA} - |
| | Chromatium vinosum |
| 110 | (P21609) Thioredoxin (TRX). {GENE: TRXA} - Clostridium |
| | litorale (Bacterium W6) |
| 111 | (P81108) Thioredoxin (TRX) (Fragment). {GENE: TRXA} - |
| | Clostridium sporogenes |
| 112 | (P81109) Thioredoxin (TRX) (Fragment). {GENE: TRXA} - |
| | Clostridium sticklandii |
| 113 | (Q9UWO2) Thioredoxin (Allergen Cop c 2). - Coprinus |
| | comatus (Shaggy mane) |
| 114 | (P29445) Thioredoxin 1. {GENE: TRXA OR TRX1} - |
| | Dictyostelium discoideum (Slime mold) |
| 115 | (P29446) Thioredoxin 2 (Fragment). {GENE: TRXB OR |
| | TRX2} - Dictyostelium discoideum (Slime mold) |
| 116 | (P29447) Thioredoxin 3. {GENE: TRXC OR TRX3} - |
| | Dictyostelium discoideum (Slime mold) |
| 117 | (P00274) Thioredoxin 1 (TRX1) (TRX). {GENE: TRXA OR |
| | TSNC OR FIPA OR B3781} - Escherichia coli, Salmonella |
| | typhimurium |
| 118 | (P52232) Thioredoxin-like protein SLR0233. {GENE: |
| | SLR0233} - Synechocystis sp. (strain PCC 6803) |
| 119 | (P33636) Thioredoxin 2 (Trx2). {GENE: TRXC OR B2582 |
| | OR Z3867 OR EC53448} - Escherichia coli, Escherichia |
| | coli O157:H7 |
| 120 | (P21610) Thioredoxin (TRX). {GENE: TRXA} - |
| | Eubacterium acidaminophilum |
| 121 | (P43785) Thioredoxin (TRX). {GENE: TRXA OR TRXM OR |
| | H10084} - Haemophilus influenzae |
| 122 | (P43787) Thioredoxin-like protein HI1115. {GENE: |
| | Hl111 5} - Haemophiius influenzae |
| 123 | (P56430) Thioredoxin (TRX). {GENE: TRXA OR HP0824 |
| | OR JHP0763} - Helicobacter pylori (Campylobacter |
| | pylori), Helicobacter pylori J99 (Campylobacter pylori |
| | J99) |
| 124 | (Q95386) Thioredoxin (EC 1.6.4.5) {GENE: TRXA} - |
| | Listeria monocytogenes |
| 125 | (Q57755) Thioredoxin. {GENE: TRX OR MJ0307} - |
| | Methanococous jannaschii |
| 126 | (P47370) Thioredoxin (TRX). {GENE: TRXA OR TRX OR |
| | MG124} - Mycoplasma genitalium |
| 127 | (P46843) Bifunctional thioredoxin-reductase/thioredoxin |
| | [Includes: Thioredoxin-reductase (EC 1.6.4.5) (TRXR); |
| | Thioredoxin]. {GENE: TRXB/A OR TRX OR ML2703} - |
| | Mycobacterium leprae |
| 128 | (P75512) Thioredoxin (TRX). {GENE: TRXA OR TRX OR |
| | MPN263 OR MP570} - Mycoplasma pneumoniae |
| 129 | (O30974) Thioredoxin (TRX). {GENE: TRXA} - |
| | Mycobacterium smegmatis |
| 130 | (P52229) Thioredoxin (TRX) (MPT46). {GENE: TRXA OR |
| | TRX OR TRXC OR RV3914 OR MT4033 OR MTVO28.05} |
| | - Mycobacterium tuberculosis |
| 131 | (P42115) Thioredoxin. {GENE: TRX} - Neurospora crassa |
| 132 | (P34723) Thioredoxin. {GENE: TRXA} - Penicillium |
| | chrysogenum |
| 133 | (Q9X2T1) Thioredoxin (TRX). {GENE: TRXA OR TRX OR |
| | PA5240} - Pseudomonas aeruginosa |
| 134 | (P10473) Thioredoxin (TRX). {GENE: TRXA} - |
| | Rhodospirillum rubrum |
| 135 | (P08058) Thioredoxin (TRX). {GENE: TRXA} - |
| | Rhodobacter sphaeroides (Rhodopseudomonas |
| | sphaeroides) |
| 136 | (Q9ZEE0) Thioredoxin (TRX). {GENE: TRXA OR RP002} |
| | - Rickettsia prowazekii |
| 137 | (P33791) Thioredoxin (TRX) (Fragment). {GENE: TRXA} - |
| | Streptomyces aureofaciens |
| 138 | (P52230) Thioredoxin (TRX). {GENE: TRXA OR |
| | SCH24.11C} - Streptomyces coelicolor |
| 139 | (Q05739) Thioredoxin (TRX). {GENE: TRXA} - |
| | Streptomyces clavuligerus |
| 140 | (P52231) Thioredoxin (TRX). {GENE: TRXA OR |
| | SLR0623} - Synechocystis sp. (strain PCC 6803) |
| 141 | (P73263) Thioredoxin-like protein SLR1139. {GENE: |
| | SLR1139} - Synechocystis sp. (strain PCC 6803) |
| 142 | (P52233) Thioredoxin (TRX). {GENE: TRXA} - |
| | Thiobacillus ferrooxidans |
| 143 | (P96132) Thioredoxin (TRX) (Fragment). {GENE: TRXA} - |
| | Thiocapsa roseopersicina |
| 144 | (P81110) Thioredoxin (TRX) (Fragment). {GENE: TRXA} - |
| | Tissierella creatinophila |
| 145 | (O83889) Thioredoxin (TRX). {GENE: TRXA OR TP0919} |
| | - Treponema pallidum |

ANIMAL THIOREDOXIN

| | |
|---|---|
| 146 | (O97680) Thioredoxin. {GENE: TXN} - Bos taurus |
| | (Bovine) |
| 147 | (Q95108) Thioredoxin, mitochondrial precursor (MT- |
| | TRX). {GENE: TXN2} - Bos taurus (Bovine) |
| 148 | (Q09433) Thioredoxin. {GENE: B0228.5} - |
| | Caenorhabditis elegans |
| 149 | (P99505) Thioredoxin (Fragment). {GENE: TXN} - Canis |
| | familiaris (Dog |
| 150 | (P08629) Thioredoxin. {GENE: TXN} - Gallus gallus |
| | (Chicken) |
| 151 | (P47938) Thioredoxin (Deadhead protein). {GENE: DHD |
| | OR CG4193} - Drosophila melanogaster (Fruit fly) |
| 152 | (P10599) Thioredoxin (ATL-derived factor) (ADF) |
| | (Surface associated sulphydryl protein) (SASP). {GENE: |
| | TXN OR TRDX OR TRX} - Homo sapiens (Human) |
| 153 | (Q99757) Thioredoxin, mitochondrial precursor (MT- |
| | TRX). {GENE: TXN2} - Homo sapiens (Human) |
| 154 | (P29451) Thioredoxin. {GENE: TXN} - Macaca mulatta |
| | (Rhesus macaque) |
| 155 | (P10639) Thioredoxin (ATL-derived factor) (ADF). {GENE: |
| | TXN} - Mus musculus (Mouse) |
| 156 | (P97493) Thioredoxin, mitochondrial precursor (MT-TRX). |
| | {GENE: TXN2} - Mus musculus (Mouse) |
| 157 | (P82460) Thioredoxin (Fragment). {GENE: TXN} - Sus |
| | scrofa (Pig) |
| 158 | (P08628) Thioredoxin. {GENE: TXN} - Oryctolagus |
| | cuniculus (Rabbit) |
| 159 | (P11232) Thioredoxin. {GENE: TXN} - Rattus norvegicus |
| | (Rat) |
| 160 | (P97615) Thioredoxin, mitochondrial precursor (MT-TRX). |
| | {GENE: TXN2 OR TRX2} - Rattus norvegicus (Rat) |
| 161 | (P50413) Thioredoxin. {GENE: TXN} - Ovis aries (Sheep) |

PLANTS THIOREDOXIN-LIKE PROTEINS

| | |
|---|---|
| 162 | (O23166) THIOL-DISULFIDE INTERCHANGE LIKE |
| | PROTEIN (THIOREDOXIN-LIKE PROTEIN) |
| | {GENE: C7A1O.160 OR AT4G37200 OR HCF164} - |
| | Arabidopsis thaliana (Mouse-ear cress) |
| 163 | (Q9C9Y6) Thioredoxin-like protein {GENE: F17O14.18} - |
| | Arabidopsis thaliana (Mouse-ear cress) |
| 164 | (Q9FYD5) Thioredoxin-like protein {GENE: F21E1_180} - |
| | Arabidopsis thaliana (Mouse-ear cress) |
| 165 | (Q38878) THIOREDOXIN-LIKE PROTEIN {GENE: TRX6 |
| | OR T7D17.3} - Arabidopsis thaliana (Mouse-ear cress) |
| 166 | (Q9LVI2) Thioredoxin-like protein - Arabidopsis thaliana |
| | (Mouse-ear cress) |
| 167 | (Q9SCN9) Thioredoxin-like protein {GENE: T4D2.150} - |
| | Arabidopsis thaliana (Mouse-ear cress) |
| 168 | (Q9SRD7) Thioredoxin-like protein, 49720-48645 |
| | {GENE: F28O16.13} - Arabidopsis thaliana (Mouse-ear |
| | cress) |
| 169 | (Q9SU84) THIOREDOXIN-LIKE PROTEIN |
| | {GENE: T16L4.180 OR AT4G29670} - Arabidopsis |
| | thaliana (Mouse-ear cress} |
| 170 | (Q9SWG6) Thioredoxin-like protein {GENE: TRX} - |
| | Hordeum bulbosum |
| 171 | (Q9SWG4) Thioredoxin-like protein {GENE: TRX} - Lolium |
| | perenne (Perennial ryegrass) |
| 172 | (Q9AS75) Thioredoxin-like protein {GENE: P0028E10.17} |
| | - Oryza sativa (Rice) |
| 173 | (O04002) CDSP32 protein (Chloroplast Drought-induced |
| | Stress Protein of 32kDa) - Solanum tuberosum (Potato) |
| 174 | (Q9SWG5) Thioredoxin-like protein {GENE: TRX} - Secale |
| | cereale (Rye) |
| 175 | (Q9SP36) Thioredoxin-like protein (Fragment) |
| | {GENE: TRX} - Secale cereale (Rye) |
| 176 | (Q9U515) Thioredoxin-like protein - Manduca sexta |
| | (Tobacco hawkmoth) (Tobacco hornworm) |

VIRUSES, BACTERIA AND FUNGI
THIOREDOXIN-LIKE PROTEINS

| | |
|---|---|
| 177 | (P43221) Thiol:disulfide interchange protein tipA |
| | (Cytochrome c biogenesis protein tlpA). {GENE: TLPA} - |
| | Bradyrhizobium japonicum |
| 178 | (P43787) Thioredoxin-like protein HI1115. {GENE: |
| | HI1115} - Haemophilus influenzae |
| 179 | (Q9GUP7) Thioredoxin-like protein {GENE: TRXLP} - |
| | Leishmania major |
| 180 | (Q9UVH0) Thioredoxin-like protein - Mortierella alpina |
| 181 | (P95355) Thioredoxin-like protein - Neisseria gonorrhoeae |
| 182 | (Q98G37) Thioredoxin-like protein {GENE: MLL3505} - |
| | Rhizobium loti (Mesorhizobium loti) |
| 183 | (P36893) Thiol:disulfide interchange protein helX |
| | precursor (Cytochrome c biogenesis protein helX). |
| | {GENE: HELX} - Rhodobacter capsulatus |
| | (Rhodopseudomonas capsulata) |
| 184 | (P52232) Thioredoxin-like protein SLR0233. {GENE: |
| | SLR0233} - Synechocystis sp. (strain PCC 6803) |
| 185 | (P73263) Thioredoxin-like protein SLR1139. {GENE: |
| | SLR1139} - Synechocystis sp. (strain PCC 6803) |
| 186 | (Q9USR1) Thioredoxin-like protein {GENE: SPBC577.08C} |
| | - Schizosaccharomyces pombe (Fission yeast) |
| 187 | (Q9R788) Thioredoxin {GENE: TPTRX} - Treponema |
| | pallidum |

ANIMALS THIOREDOXIN-LIKE PROTEINS

| | |
|---|---|
| 188 | (Q9UAV4) F46E10.9 PROTEIN (THIOREDOXIN-LIKE |
| | PROTEIN DPY-11) {GENE: F46E10.9 OR DPY-11} - |
| | Caenorhabditis elegans |
| 189 | (Q9N2K6) Thioredoxin-like protein (Y54E10A.3 protein) |
| | (Thioredoxin-like protein TXL) {GENE: TXL OR |
| | Y54E10A.3} - Caenorhabditis elegans |
| 190 | (Q9VRP3) THIOREDOXIN-LIKE PROTEIN TXL (CG5495 |
| | PROTEIN) {GENE: TXL OR CG5495} - Drosophila |
| | melanogaster (Fruit fly) |
| 191 | (O43396) Thioredoxin-like protein (32 kDa thioredoxin- |
| | related protein). {GENE: TXNL OR TRP32 OR TXL} - |
| | Homo sapiens (Human) |
| 192 | (O76003) Thioredoxin-like protein - Homo sapiens |
| | (Human) |
| 193 | (Q9S753) THIOREDOXIN-LIKE PROTEIN {GENE: TRX} - |
| | Phalaris coerulescens |
| 194 | (O77404) TRYPAREDOXIN - Trypanosoma brucei brucei |

PLANT THIOREDOXIN-REDUCTASES

| | |
|---|---|
| 195 | (Q39243) Thioredoxin-reductase 1 (EC 1.6.4.5) (NADPH- |
| | dependent thioredoxin-reductase 1) (NTR 1). {GENE: |
| | NTR1 OR AT4G35460 OR F15J1.30} - Arabidopsis |
| | thaliana (Mouse-ear cress) |
| 196 | (Q39242) Thioredoxin-reductase 2 (EC 1.6.4.5) (NADPH- |
| | dependent thioredoxin-reductase 2) (NTR 2). {GENE: |
| | NTR2 OR AT2G17420 OR F5J6.18} - Arabidopsis |
| | thaliana (Mouse-ear cress) |

VIRUSES, BACTERIA AND FUNGI
THIOREDOXIN-REDUCTASES

| | |
|---|---|
| 197 | (O66790) Thioredoxin-reductase (EC 1.6.4.5) |
| | (TRXR). {GENE: TRXB OR AQ_500} - Aquifex aeolicus |
| 198 | (P80880) Thioredoxin-reductase (EC 1.6.4.5) (TRXR) |
| | (General stress protein 35) (GSP35). {GENE: TRXB} - |
| | Bacillus subtilis |
| 199 | (P94284) Thioredoxin-reductase (EC 1.6.4.5) (TRXR). |
| | {GENE: TRXB OR BB0515} - Borrelia burgdorferi (Lyme |
| | disease spirochete) |
| 200 | (P57399) Thioredoxin-reductase (EC 1.6.4.5) (TRXR). |
| | {GENE: TRXB OR BU314} - Buchnera aphidicola (subsp. |
| | Acyrthosiphon pisum) (Acyrthosiphon pisum symbiotic |
| | bacterium) |
| 201 | (P81433) Thioredoxin-reductase (EC 1.6.4.5) (TRXR). |
| | {GENE: TRXB} - Buchnera aphidicola (subsp. Schizaphis |
| | graminum) |
| 202 | (Q9PKT7) Thioredoxin-reductase (EC 1.6.4.5) (TRXR). |
| | {GENE: TRXB OR TC0375} - Chlamydia muridarum |
| 203 | (Q9Z8M4) Thioredoxin-reductase (EC 1.6.4.5) (TRXR). |
| | {GENE: TRXB OR CPN0314 OR CP0444} - Chlamydia |
| | pneumoniae (Chlamydophila pneumoniae) |
| 204 | (084101) Thioredoxin-reductase (EC 1.6.4.5) (TRXR). |
| | {GENE: TRXB OR CT099} - Chlamydia trachomatis |
| 205 | (P52213) Thioredoxin-reductase (EC 1.6.4.5) (TRXR). |
| | {GENE: TRXB} - Clostridium litorale (Bacterium W6) |
| 206 | (P39916) Thioredoxin-reductase (EC 1.6.4.5) (TRXR). |
| | {GENE: TRXB} - Coxiella burnetii |
| 207 | (P09625) Thioredoxin-reductase (EC 1.6.4.5) (TRXR). |
| | {GENE: TRXB OR B0888 OR Z1232 OR ECS0973} - |
| | Escherichia coli, Escherichia coli O157:H7 |
| 208 | (P50971) Thioredoxin-reductase (EC 1.6.4.5) (TRXR). |
| | {GENE: TRXB} - Eubacterium acidaminophilum |
| 209 | (P43788) Thioredoxin-reductase (EC 1.6.4.5) (TRXR). |
| | {GENE: TRXB OR HIll 58} - Haemophiius influenzae |
| 210 | (Q9ZL18) Thioredoxin-reductase (EC 1.6.4.5) (TRXR). |
| | {GENE: TRXB OR JHP0764} - Helicobacter pylori J99 |
| | (Campylobacter pylori J99) |
| 211 | (P56431) Thioredoxin-reductase (EC 1.6.4.5) (TRXR). |
| | {GENE: TRXB OR HP0825} - Helicobacter pylori |
| | (Campylobacter pylori) |
| 212 | (O32823) Thioredoxin-reductase (EC 1.6.4.5) (TRXR). |
| | {GENE: TRXB OR LMO2478} - Listeria monocytogenes |
| 213 | (P47348) Thioredoxin-reductase (EC 1.6.4.5) (TRXR). |
| | {GENE: TRXB OR MG102} - Mycoplasma genitalium |
| 214 | (P46843) Bifunctional thioredoxin-reductase/thioredoxin |
| | [Includes: Thioredoxin-reductase (EC 1.6.4.5) (TRXR); |
| | Thioredoxin]. {GENE: TRXB/A OR TRX OR ML2703} - |
| | Mycobacterium leprae |
| 215 | (P75531) Thioredoxin-reductase (EC 1.6.4.5) (TRXR). |
| | {GENE: TRXB OR MPN240 OR MP591} - Mycoplasma |
| | pneumoniae |
| 216 | (O30973) Thioredoxin-reductase (EC 1.6.4.5) (TRXR). |
| | {GENE: TRXB} - Mycobacterium smegmatis |
| 217 | (P52214) Thioredoxin-reductase (EC 1.6.4.5) (TRXR) |
| | (TR). {GENE: TRXB OR RV3913 OR MT4032 OR |
| | MTV028.04} - Mycobacterium tuberculosis |
| 218 | (P51978) Thioredoxin-reductase (EC 1.6.4.5). {GENE: |
| | CYS-9} - Neurospora crassa |
| 219 | (P43496) Thioredoxin-reductase (EC 1.6.4.5). {GENE: |
| | TRXB} - Penicillium chrysogenum |
| 220 | (Q9ZD97) Thioredoxin-reduotase (EC 1.6.4.5) (TRXR). |
| | {GENE: TRXB OR RP445} - Rickettsia prowazekii |
| 221 | (Q92375) Thioredoxin-reductase (EC 1.6.4.5). {GENE: |
| | SPBC3F6.03} - Schizosacoharomyces pombe (Fission |
| | yeast) |
| 222 | (Q05741) Thioredoxin-reductase (EC 1.6.4.5) (TRXR). |
| | {GENE: TRXB} - Streptomyces clavuligerus |
| 223 | (P52215) Thioredoxin-reductase (EC 1.6.4.5) (TRXR). |
| | {GENE: TRXB OR SCH24.12} - Streptomyces coelicolor |
| 224 | (O83790) Thioredoxin-reductase (EC 1.6.4.5) (TRXR). |
| | {GENE: TRXB OR TP0814} - Treponema pallidum |
| 225 | (P80892) Thioredoxin-reductase (EC 1.6.4.5) (TRXR) |
| | (Fragment). {GENE: TRXB} - Vibrio fischeri |
| 226 | (P29509) Thioredoxin-reductase 1 (EC 1.6.4.5). {GENE: |
| | TRR1 OR YDR353W OR D9476.5} - Saccharomyces |
| | cerevisiae (Baker's yeast) |
| 227 | (P38816) Thioredoxin-reductase 2, mitochondrial |
| | precursor (EC 1.6.4.5). {GENE: TRR2 OR YHR106W} - |
| | Sacoharomyces cerevisiae (Baker's yeast) |

ANIMAL THIOREDOXIN-REDUCTASES

| | |
|---|---|
| 228 | (O62768) Thioredoxin-reductase (EC 1.6.4.5). {GENE: |
| | TXNRD1} - Bos taurus (Bovine) |
| 229 | (Q17745) Thioredoxin-reductase (EC 1.6.4.5). {GENE: |
| | C06G3.7} - Caenorhabditis elegans |
| 230 | (Q16881) Thioredoxin-reductase (EC 1.6.4.5). {GENE: |
| | TXNRD1} - Homo sapiens (Human) |
| 231 | (Q25861) Thioredoxin-reductase (EC 1.6.4.5) (TrxR). |
| | {GENE: TR OR GR} - Plasmodium falciparum (isolate FCH |
| | 5) |

Other thioredoxin-reductases

PLANTS THIOREDOXIN-REDUCTASES

| | |
|---|---|
| 232 | (O22229) Thioredoxin-reductase {GENE: AT2G41680} - |
| | Arabidopsis thaliana (Mouse-ear cress) |
| 233 | (Q39951) NADPH thioredoxin-reductase (Fragment) - |
| | Helianthus annuus (Common sunflower) |

VIRUSES, BACTERIA AND FUNGI
THIOREDOXIN-REDUCTASES

| | |
|---|---|
| 234 | (O28718) THioredoxin-reductase (TRXB) {GENE: AF1554} |
| | - Archaeoglobus fulgidus |
| 235 | (Q9K703) Thioredoxin-reductase (NADPH) (EC 1.6.4.5) |
| | {GENE: TRXB OR BH3571} - Bacillus halodurans |
| 236 | (Q9K7F3) Thioredoxin-reductase {GENE: BH3408} - |
| | Bacillus halodurans |
| 237 | (Q9KCZ0) Thioredoxin-reductase {GENE: BH1429} - |
| | Bacillus halodurans |
| 238 | (Q9KCZ1) Thioredoxin-reductase {GENE: BH1428} - |
| | Bacillus halodurans |
| 239 | (Q9PIY1) Thioredoxin-reductase (EC 1.6.4.5) |
| | {GENE: TRXB OR CJ0146} - Campylobacter jejuni |
| 240 | (Q9A4G3) Thioredoxin-reductase {GENE: CC2871} - |
| | Caulobacter crescentus |
| 241 | (Q97EM8) Thioredoxin-reductase {GENE: CAC3082} - |
| | Clostridium acetobutylicum |
| 242 | (Q97IU2) Thioredoxin-reductase {GENE: CAC1548} - |
| | Clostridium acetobutylicum |
| 243 | (Q9EV96) Thioredoxin-reductase {GENE: TRXB} - |
| | Clostridium sticklandii |
| 244 | (Q9RSY7) THioredoxin-reductase {GENE: DR1982} - |
| | Deinococcus radiodurans |
| 245 | (O30739) Thioredoxin-reductase (Fragment) - |
| | Enterococcus faecalis (Streptococcus faecalis) |
| 246 | (O54535) Thioredoxin-reductase {GENE: TRXB OR |
| | TRXB1_2 OR VNG6452G OR TRXB1_1 OR |
| | VNG6O74G} - Halobacterium sp. (strain NRC-1) |
| | [Plasmid pNRC100, and Plasmid pNRC200] |
| 247 | (P82854) Thioredoxin-reductase (EC 1.6.4.5) |
| | {GENE: TRXB2} - Halobacterium sp. (strain NRC-1) |
| 248 | (Q9HN08) Thioredoxin-reductase {GENE: TXRB3 OR |
| | VNG2310G} - Halobacterium sp. (strain NRC-1) |
| 249 | (O25779) THioredoxin-reductase (TRXB) {GENE: HP1164} |
| | - Helicobacter pylori (Campylobacter pylori) |
| 250 | (O86255) Thioredoxin-reductase {GENE: TRXB} - |
| | Klebsiella oxytoca |
| 251 | (Q9AEV9) Thioredoxin-reductase (Fragment) |
| | {GENE: TRXB} - Lactococcus lactis (subsp. lactis) |
| | (Streptococcus lactis) |
| 252 | (Q9CF34) Thioredoxin-reductase (EC 1.6.4.5) |
| | {GENE: TRXB2} - Lactococcus lactis (subsp. lactis) |
| | (Streptococcus lactis) |
| 253 | (Q9CH02) Thioredoxin-reductase (EC 1.6.4.5) |
| | {GENE: TRXB1} - Lactococcus lactis (subsp. lactis) |
| | (Streptococcus lactis) |
| 254 | (Q9ZFC8) Thioredoxin-reductase (Fragment) |
| | {GENE: TRXB} - Lactococcus lactis |
| 255 | (O32822) Hypothetical 39.7 kDa protein (Fragment) - |
| | Listeria monocytogenes |
| 256 | (O26804) THioredoxin-reductase {GENE: MTH708} - |
| | Methanothermobacter thermautotrophicus |
| 257 | (P94397) Homologue of thioredoxin-reductase of |
| | Mycoplama genitalium {GENE: YCGT} - Bacillus subtilis |
| 258 | (Q98PK9) THioredoxin-reductase (EC 1.6.4.5) |
| | {GENE: MYPU_7130} - Mycoplasma pulmonis |
| 259 | (Q9JU23) Thioredoxin-reductase (EC 1.6.4.5) |
| | {GENE: TRXB OR NMA1538} - Neisseria meningitidis |
| | (serogroup A) |
| 260 | (Q9JZ28) Thioredoxin-reductase {GENE: NMB1324} - |
| | Neisseria meningitidis (serogroup B) |
| 261 | (Q910M2) Thioredoxin-reductase 1 {GENE: TRXB1 OR |
| | PA2616} - Pseudomonas aeruginosa |
| 262 | (Q91592) Thioredoxin-reductase 2 {GENE: TRXB2 OR |
| | PA0849} - Pseudomonas aeruginosa |
| 263 | (Q9VOQ8) THioredoxin-reductase (TRXB) {GENE: TRXB |
| | OR PAB0500} - Pyrococcus abyssi |
| 264 | (Q9ZD33) THioredoxin-reductase (TRXB2) {GENE: RP514} |
| | - Rickettsia prowazekii |
| 265 | (O54079) Thioredoxin-reductase (EC 1.6.4.5) |
| | {GENE: TRXB} - Staphylococcus aureus |
| 266 | (Q9RIS2) Thioredoxin-reductase {GENE: TRXB OR |
| | TRXB2} - Streptomyces coelicolor |
| 267 | (Q9K4L6) Thioredoxin-reductase {GENE: SC5F8.08C} - |
| | Streptomyces coelicolor |
| 268 | (Q97PY2) Thioredoxin-reductase {GENE: SP1458} - |
| | Streptococcus pneumoniae |
| 269 | (Q9A0B5) Thioredoxin-reductase {GENE: SPY0850} - |
| | Streptococcus pyogenes |
| 270 | (Q97V69) Thioredoxin-reductase (trxB-2) (EC 1.6.4.5) |
| | {GENE: TRXB-2} - Sulfolobus solfataricus |
| 271 | (Q97W27) Thioredoxin-reductase (trxB-3) (EC 1.6.4.5) |
| | {GENE: TRXB-3} - Sulfolobus solfataricus |
| 272 | (Q97WJ5) Thioredoxin-reductase (trxB-1) (EC 1.6.4.5) |
| | {GENE: TRXB-1} - Sulfolobus solfataricus |
| 273 | (Q98159) Thioredoxin-reductase {GENE: MLL2552} - |
| | Rhizobium loti (Mesorhizobium loti) |
| 274 | (Q98M06) Thioredoxin-reductase {GENE: MLL0792} - |
| | Rhizobium loti (Mesorhizobium loti) |
| 275 | (Q9UR80) 35 kDa THioredoxin-reductase HOMOLOG |
| | (FRAGMENT) {GENE: TRR1 AND YDR353W} - |
| | Saccharomyces cerevisiae (Baker's yeast) |
| 276 | (Q9ZEH4) THIOREDOXIN {GENE: TRXA OR SA0992} - |
| | Staphylococcus aureus, Staphylococcus aureus subsp. |
| | aureus N315 |
| 277 | (Q9S1H1) Thioredoxin-reductase (Fragment) |
| | {GENE: TRXB} - Staphylococcus xylosus |
| 278 | (Q9HJI4) Thioredoxin-reductase {GENE: TA0984} - |
| | Thermoplasma acidophilum |
| 279 | (Q9WZX3) THioredoxin-reductase {GENE: TM0869} - |
| | Thermotoga maritima |
| 280 | (Q979K8) Thioredoxin-reductase {GENE: TVG1183005} - |
| | Thermoplasma volcanium |
| 281 | (Q9PR71) Thioredoxin-reductase {GENE: TRXB OR |
| | UU074} - Ureaplasma parvum (Ureaplasma urealyticum |
| | biotype 1) |
| 282 | (Q9KSS4) Thioredoxin-reductase {GENE: VC11821 - |
| | Vibrio cholerae |
| 283 | (Q9PDD1) Thioredoxin-reductase {GENE: XF1448} - |
| | Xylella fastidiosa |
| 284 | (Q9X5F7) Thioredoxin-reductase {GENE: TRXB1} - |
| | Zymomonas mobilis |

ANIMAL THIOREDOXIN-REDUCTASES

| | |
|---|---|
| 285 | (Q9GKW9) Thioredoxin-reductase 3 (Fragment) |
| | {GENE: TRXR3} - Bos taurus (Bovine) |
| 286 | (Q9N2I8) Thioredoxin-reductase (EC 1.6.4.5) - Bos taurus |
| | (Bovine) |
| 287 | (Q9N2K1) Thioredoxin-reductase homolog - |
| | Caenorhabditis elegans |
| 288 | (Q9NJH3) Thioredoxin-reductase - Caenorhabditis |
| | elegans |
| 289 | (Q9VNT5) CG11401 PROTEIN (THioredoxin-reductase 2) |
| | {GENE: TRXR-2 OR CG11401} - Drosophila melanogaster |
| | (Fruit fly) |
| 290 | (O95840) Thioredoxin-reductase - Homo sapiens (Human) |
| 291 | (Q9UES8) Thioredoxin-reductase GRIM-12 - Homo |
| | sapiens (Human) |
| 292 | (Q9UH79) Thioredoxin-reductase {GENE: TR} - Homo |
| | sapiens (Human) |
| 293 | (Q9UQU8) Thioredoxin-reductase - Homo sapiens |
| | (Human) |
| 294 | (Q9NNW6) Thioredoxin-reductase TR2 (Fragment) - |
| | Homo sapiens (Human) |
| 295 | (Q9NNW7) Thioredoxin-reductase TR3 - Homo sapiens |
| | (Human) |
| 296 | (Q9P101) Thioredoxin-reductase 3 (Fragment) |
| | {GENE: TRXR3} - Homo sapiens (Human) |
| 297 | (Q9P2Y0) Thioredoxin-reductase II beta (EC 1.6.4.5) - |
| | Homo sapiens (Human) |
| 298 | (Q9H2Z5) Mitochondrial thioredoxin-reductase |
| | {GENE: TRXR2A} - Homo sapiens (Human) |
| 299 | (Q99475) KM-102-DERIVED REDUCTASE-LIKE FACTOR |
| | (THioredoxin-reductase) - Homo sapiens (Human) |
| 300 | (Q99P49) Thioredoxin-reductase 1 {GENE: TXNRD1} - |
| | Mus musculus (Mouse) |
| 301 | (Q9CSV5) Thioredoxin-reductase 1 (Fragment) |
| | {GENE: TXNRD1} - Mus musculus (Mouse) |
| 302 | (Q9CZE5) Thioredoxin-reductase 1 {GENE: TXNRD1} - |
| | Mus musculus (Mouse) |
| 303 | (Q9JHA7) Thioredoxin-reductase TR3 {GENE: TXNRD2 |
| | OR TR3} - Mus musculus (Mouse) |
| 304 | (Q9JLT4) Thioredoxin-reductase {GENE: TXNRD2 OR |
| | TRXR2} - Mus musculus (Mouse) |
| 305 | (Q9JMH5) Thioredoxin-reductase 2 {GENE: TXNRD2 OR |
| | TXNRD2} - Mus musculus (Mouse) |
| 306 | (Q9JMH6) Thioredoxin-reductase 1 {GENE: TXNRD1 OR |
| | TXNRD1} - Mus musculus (Mouse) |
| 307 | (O89049) Thioredoxin-reductase - Rattus norvegicus |
| | (Rat) |
| 308 | (Q9JKZ3) Thioredoxin-reductase 1 (Fragment) - Rattus |
| | norvegicus (Rat) |
| 309 | (Q9JKZ4) Thioredoxin-reductase 1 - Rattus norvegicus |
| | (Rat) |
| 310 | (Q9JLE6) Thioredoxin-reductase (Fragment) - Rattus |
| | norvegicus (Rat) |
| 311 | (Q9R1I3) NADPH-dependent thioredoxin-reductase |
| | {GENE: TRR1} - Rattus norvegicus (Rat) |
| 312 | (Q9Z0J5) Thioredoxin-reductase precursor |
| | {GENE: TRXR2} - Rattus norvegicus (Rat) |
| 313 | (Q9MYY8) Redox enzyme thioredoxin-reductase - Sus |
| | scrofa (Pig) |

What is claimed is:

1. A method of producing an oil body associated with a recombinant multimeric-protein-complex, said method comprising:
(a) producing in a cell comprising oil bodies, a first recombinant polypeptide and a second recombinant polypeptide wherein said first recombinant polypeptide is capable of associating with said second recombinant polypeptide to form said multimeric-protein-complex; and
(b) associating said multimeric-protein-complex with an oil body through an oil-body-targeting-protein capable of associating with said oil body and said first recombinant polypeptide,
wherein said first recombinant polypeptide is a thioredoxin and said second recombinant polypeptide is a thioredoxin-reductase.

2. A method of expressing a recombinant multimeric-protein-complex comprising a first and second recombinant polypeptide in a cell, said method comprising:
(a) introducing into a cell a first chimeric nucleic acid sequence comprising:
(i) a first nucleic acid sequence capable of regulating transcription in said cell operatively linked to;
(ii) a second nucleic acid sequence encoding a first recombinant polypeptide;
(b) introducing into said cell a second chimeric nucleic acid sequence comprising:
(i) a third nucleic acid sequence capable of regulating transcription in said cell operatively linked to;
(ii) a fourth nucleic acid sequence encoding a second recombinant polypeptide;
(c) growing said cell under conditions to permit expression of said first and second recombinant polypeptide in a progeny cell comprising oil bodies wherein said first recombinant polypeptide and said second recombinant polypeptide are capable of forming a multimeric-protein-complex; and
(d) associating said first recombinant polypeptide with an oil body through an oil-body-targeting-protein capable of associating with said oil body and said first recombinant polypeptide,
wherein said first recombinant polypeptide is a thioredoxin and said second recombinant polypeptide is a thioredoxin-reductase.

3. A method of producing in a plant a recombinant multimeric-protein-complex, said method comprising:
(a) preparing a first plant comprising cells, said cells comprising oil bodies and a first recombinant polypeptide wherein said first recombinant polypeptide is capable of associating with said oil bodies through an oil-body-targeting-protein;
(b) preparing a second plant comprising cells, said cells comprising oil bodies and a second recombinant polypeptide; and
(c) sexually crossing said first plant with said second plant to produce a progeny plant comprising cells, said cells comprising oil bodies, wherein said oil bodies are capable of associating with said first recombinant polypeptide, and said first recombinant recombinant polypeptide is capable of associating with said second recombinant polypeptide to form said recombinant multimeric-protein-complex,
wherein said first recombinant polypeptide is a thioredoxin and said second recombinant polypeptide is a thioredoxin-reductase.

4. A chimeric nucleic acid sequence encoding a multimeric-fusion-protein, said nucleic acid comprising:
(a) a first nucleic acid sequence encoding an oil-body-targeting-protein operatively linked in reading frame to;
(b) a second nucleic acid sequence encoding a first recombinant polypeptide; linked in reading frame to;
(c) a third nucleic acid sequence encoding a second recombinant polypeptide, wherein said first and second recombinant polypeptide are capable of forming a multimeric-protein-complex, and
wherein said first recombinant polypeptide is a thioredoxin and said second recombinant polypeptide is a thioredoxin-reductase.

5. A recombinant multimeric-fusion-protein comprising (i) an oil-body-targeting-protein, or fragment thereof, (ii) a first recombinant polypeptide and a (iii) second recombinant polypeptide, wherein said first and second recombinant polypeptides are capable of forming a multimeric-protein-complex, and
wherein said first recombinant polypeptide is a thioredoxin and said second recombinant polypeptide is a thioredoxin-reductase.

6. Isolated oil bodies comprising a multimeric-protein-complex comprising (i) an oil-body-targeting-protein and (ii) a first recombinant polypeptide, said oil bodies further comprising a second recombinant polypeptide, wherein said first and second recombinant polypeptide are capable of forming a multimeric-protein-complex, and
wherein said first recombinant polypeptide is a thioredoxin and said second recombinant polypeptide is a thioredoxin-reductase.

7. Isolated oil bodies comprising
(a) a first fusion protein comprising a first oil-body-targeting-protein fused to a first recombinant polypeptide; and
(b) a second fusion protein comprising a second oil-body-targeting-protein fused to a second recombinant polypeptide,
wherein said first and second recombinant polypeptide are capable of forming a multimeric-protein-complex, and
wherein said first recombinant polypeptide is a thioredoxin and said second recombinant polypeptide is a thioredoxin-reductase.

8. A cell comprising oil bodies and (i) an oil-body-targeting-protein, (ii) a first recombinant polypeptide and (iii) a second recombinant polypeptide wherein
(1) said first recombinant polypeptide is capable of associating with said oil-body-targeting-protein; and
(2) said first recombinant polypeptide capable of associating with said second recombinant polypeptide to form a multimeric-protein-complex,
wherein said first recombinant polypeptide is a thioredoxin and said second recombinant polypeptide is a thioredoxin-reductase.

9. A composition comprising isolated oil bodies, thioredoxin and thioredoxin-reductase.

10. A food product, personal care product or pharmaceutical composition comprising the composition of claim 9.

11. The personal care product of claim 10 wherein said personal care product reduces the oxidative stress to the surface area of the human body or is used to lighten the skin.

12. A method of reducing allergenicity of a food comprising the steps of:
providing the isolated oil bodies of claim 7; and
adding the isolated oil bodies to the food, whereby allergenicity of the food is reduced.

13. A method of treating or protecting a target against oxidative stress, comprising the steps of:
providing the recombinant fusion polypeptide of claim 3; and
contacting the recombinant fusion polypeptide with a target, wherein the target is susceptible to oxidative stress, thereby treating or protecting against the stress.

14. A method for preparing an enzymatically active redox protein associated with oil bodies comprising:
a) producing in a cell a redox fusion polypeptide comprising a first redox protein linked to a second redox protein;
b) associating said redox fusion polypeptide with oil bodies through an oil-body-targeting-protein capable of associating with said redox fusion polypeptide and said oil bodies; and
c) isolating said oil bodies associated with said redox fusion polypeptide,
wherein said first redox protein is a thioredoxin and said second redox protein is a thioredoxin-reductase.

15. A method for preparing a redox protein associated with oil bodies comprising:
a) introducing into a cell a chimeric nucleic acid sequence comprising:
1) a first nucleic acid sequence capable of regulating transcription in said cell operatively linked to;
2) a second nucleic acid sequence encoding a recombinant fusion polypeptide comprising (i) a nucleic acid sequence encoding a sufficient portion of an oil-body-protein to provide targeting of said recombinant fusion polypeptide to an oil body linked to (ii) a nucleic acid sequence encoding a redox fusion polypeptide comprising a first redox protein linked to a second redox protein operatively linked to;
3) a third nucleic acid sequence capable of terminating transcription in said cell;
b) growing said cell under conditions to permit expression of said redox fusion polypeptide in a progeny cell comprising oil bodies; and
c) isolating from said progeny cell said oil bodies comprising said redox fusion polypeptide,
wherein said first redox protein is a thioredoxin and said second redox protein is a thioredoxin-reductase.

16. A chimeric nucleic acid comprising:
1) a first nucleic acid sequence capable of regulating transcription in a host cell operatively linked to;
2) a second nucleic acid sequence encoding a recombinant fusion polypeptide comprising (i) a nucleic acid sequence encoding a sufficient portion of an oil-body-protein to provide targeting of said recombinant fusion polypeptide to an oil body linked to (ii) a nucleic acid sequence encoding a redox fusion polypeptide comprising a first redox protein linked to a second redox protein operatively linked to;
3) a third nucleic acid sequence capable of terminating transcription in said cell,
wherein said first redox protein is a thioredoxin and said second redox protein is a thioredoxin-reductase.

17. A transgenenic plant comprising the chimeric nucleic acid sequence of claim 16.

18. A plant seed comprising the chimeric nucleic acid of claim 16.

19. A safflower seed comprising the chimeric nucleic acid of claim 16.

20. A nucleic acid construct comprising a gene fusion, wherein the gene fusion comprises a first region encoding an oil-body-protein or an active fragment thereof, operably linked to a second region encoding at least one thioredoxin-related protein or an active fragment thereof,
wherein the at least one thioredoxin-related protein is selected from thioredoxin or thioredoxin-reductase.

21. A transgenic plant containing a nucleic acid construct comprising a gene fusion, wherein the gene fusion comprises a region encoding an oil-body-protein or an active fragment thereof, operably linked to a region encoding a first thioredoxin-related protein or an active fragment thereof,
wherein the thioredoxin-related protein is selected from thioredoxin or thioredoxin-reductase.

22. A transgenic plant comprising a nucleic acid construct comprising a seed-specific promoter operably linked to a gene fusion, wherein the gene fusion comprises a region encoding an oil-body-protein or an active fragment thereof, operably linked to a region encoding a first thioredoxin-related protein or an active fragment thereof, wherein a fusion protein comprising activities of oleosin and the thioredoxin-related protein is produced in a seed of the plant,
wherein the thioredoxin-related protein is selected from thioredoxin or thioredoxin-reductase.

23. A method of making a fusion protein comprising a thioredoxin-related activity, the method comprising the steps of:
providing a transgenic plant comprising a nucleic acid construct comprising a seed-specific promoter operably linked to a gene fusion, wherein the gene fusion comprises a region encoding an oil-body-protein or an active fragment thereof, operably linked to a region encoding a first thioredoxin-related protein or an active fragment thereof, the gene fusion encoding a fusion protein comprising a thioredoxin-related activity;
obtaining seeds from the plant; and
recovering the fusion protein by isolating oil bodies from the seeds,
wherein the thioredoxin-related protein is selected from thioredoxin or thioredoxin-reductase.

24. A method of reducing allergenicity of a food comprising the steps of:
providing a preparation comprising oil bodies associated with a fusion protein, the fusion protein comprising an oil-body-protein or an active fragment thereof and a thioredoxin-related protein or an active fragment thereof; and
adding the preparation to the food, whereby allergenicity of the food is reduced due to activity of the thioredoxin-related protein or fragment,
wherein the thioredoxin-related protein is selected from thioredoxin or thioredoxin-reductase.

25. A composition comprising a fusion protein, the fusion protein comprising an oil-body-protein or an active fragment thereof and a thioredoxin-related protein or an active fragment thereof, in a pharmaceutically acceptable carrier,
wherein the thioredoxin-related protein is selected from thioredoxin or thioredoxin-reductase.

26. A cosmetic formulation comprising oil bodies associated with a fusion protein, the fusion protein comprising an oil-body-protein or an active fragment thereof and a thioredoxin-related protein or an active fragment thereof, in an acceptable carrier,
wherein the thioredoxin-related protein is selected from thioredoxin or thioredoxin-reductase.

27. A method of treating or protecting a target against oxidative stress, comprising the steps of:
providing a preparation comprising a fusion protein, the fusion protein comprising an oil-body-protein or an active fragment thereof and a thioredoxin-related protein or an active fragment thereof; and
contacting the preparation with a target, wherein the target is susceptible to oxidative stress, thereby treating or protecting against the stress,
wherein the thioredoxin-related protein is selected from thioredoxin or thioredoxin-reductase.

28. A nucleic acid construct comprising a gene fusion, wherein the gene fusion comprises a first region encoding an oil-body-protein or an active fragment thereof, operably linked to a second region encoding at least one recombinant polypeptide and an oil-body-surface-avoiding linker in frame between the first and second region polypeptides.
